(19) United States
(12) Patent Application Publication
(10) Pub. No.: US 2003/0022286 A1
(43) Pub. Date: Jan. 30, 2003

(54) Novel transporter-like genes and uses therefor

(75) Inventor: Rory A.J. Curtis, Southborough, MA (US)

Correspondence Address: Millennium Pharmaceuticals, Inc., 75 Sidney Street, Cambridge, MA, 02139, US

(73) Assignee: Millennium Pharmaceuticals, Inc.

(21) Appl. No.: 10/060,763

(22) Filed: Jan. 30, 2002

Related U.S. Application Data
(63) Continuation of application No. 09/365,162, filed on Jul. 30, 1999, now abandoned.

(51) Int. Cl. C07K 14/435; C07H 21/04; C12P 21/02; C12N 5/06

(52) U.S. Cl. 435/69.1; 435/320.1; 435/325; 530/350; 536/23.5

(57) ABSTRACT

The invention provides isolated nucleic acids encoding transmembrane transport proteins and fragments, derivatives, and variants thereof. These nucleic acids and proteins are useful for diagnosis, prevention, and therapy of a number of human and other animal disorders. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides, and antibodies. Diagnostic, screening, and therapeutic methods utilizing compositions of the invention are also provided. The nucleic acids and polypeptides of the present invention are useful as modulating agents in regulating a variety of cellular processes relating to transmembrane transport of charged organic compounds such as prostaglandins and thromboxanes.

### BACKGROUND OF THE INVENTION

Cell membranes form a semi-permeable barrier which surrounds the cytoplasm. The hydrophobic character of the lipids of the cell membrane act as a barrier which generally prevents diffusion of water and water-soluble substances between the intracellular and extracellular fluid environments. Although lipid-soluble substances (e.g. oxygen, carbon dioxide, various alcohols, and the like) may enter and leave a cell by simple diffusion through the cell membrane, most other substances can traverse the cell membrane substantially only by way of one or more transport proteins. These transport proteins are integral membrane proteins, and facilitate transmembrane transport by a variety of mechanisms including, for example, formation of pores through which molecules and ions (especially small molecules) can diffuse, facilitated diffusion, diffusion through gated channels, and active transport.

Large molecules (e.g. those comprising more than a few atoms), and especially charged large molecules are generally not able to cross the cytoplasmic membrane in the absence of a transport protein. For many of these large molecules, transport proteins occur in the membrane, which are adapted to specifically transport one or more of a class of molecules. Exemplary classes of molecules include prostaglandins, thromboxanes, hexoses, disaccharides, hormones (e.g. insulin), peptides, neurotransmitters, cytokines, chemokines, and the like.

Prostaglandins and thromboxanes are a group of compounds derived from unsaturated fatty acids (primarily arachidonic acid via the cyclooxygenase pathway). These compounds are potent mediators of a diverse group of physiological processes and disorders including, but not limited to glaucoma, ovum fertilization, sperm motility, pregnancy, labor, delivery, abortion, gastric protection, peptic ulcer formation, intestinal fluid secretion, liver protection, liver damage, liver fibrosis, pain stimulation, neural transmission disorders, stroke, regeneration of chronically or traumatically damaged neuronal structures, developmental neuronal disorders, neuronal cancers, peripheral nerve deficit, coronary insufficiency, angina, glomerular filtration, maintenance of body temperature, fever, airway resistance, asthma, chronic obstructive pulmonary disorder, modulation of blood pressure, hypertension, shock, modulation of inflammation, platelet aggregation, abnormal blood coagulation, atherosclerosis, arteriosclerosis, and coronary artery disease.

Known prostaglandins and thromboxanes include prostaglandins A₁, A₂, B₁, B₂, D₂, E₁, E₂, F_{1α}, F_{2α}, G₂, H₂, I₂, and J₂ and thromboxanes A₂ and B₂. Prostaglandins are negatively charged at physiological pH, and thus traverse biological membranes only poorly, if at all. Transmembrane transport of prostaglandins appears to be mediated by a carrier in at least lung, choroid plexus, liver, eye, vagina, uterus, and placental tissues. cDNAs encoding rat and human prostaglandin transmembrane transporters have been isolated (Jacquemin et al. (1994) Proc. Natl. Acad. Sci. USA 91:133; Kanai et al. (1995) Science 268:866-869; U.S. Pat. No. 5,792,851). These two proteins comprise twelve transmembrane domains and exhibit about 37% amino acid sequence identity. However, expression of each of these two proteins has been associated with transmembrane transport of only certain of the known prostaglandins. Clearly, there should be proteins which modulate transmembrane transport of prostaglandins and thromboxanes which are not transported by the prostaglandin transporters already described in the art. However, such additional proteins have not been described in the art.

The present invention provides nucleotide and amino acid sequence information corresponding to proteins which catalyze or facilitate transmembrane transport of charged organic compounds such as prostaglandins and thromboxanes in a variety of tissues.

### SUMMARY OF THE INVENTION

The present invention is based, at least in part, on discovery of human cDNA molecules which encode proteins which are herein designated 65h2 and 593. These proteins catalyze or facilitate transmembrane transport of charged organic compounds such as one or more of prostaglandins, thromboxanes, hexoses, disaccharides, hormones (e.g. insulin), peptides, neurotransmitters, cytokines, chemokines, and the like. These two proteins, fragments thereof, derivatives thereof, and variants thereof are collectively referred to herein as the polypeptides of the invention or the proteins of the invention. Nucleic acid molecules encoding polypeptides of the invention are collectively referred to as nucleic acids of the invention.

The nucleic acids and polypeptides of the present invention are useful as modulating agents in regulating a variety of cellular processes, particularly including processes which involve transmembrane transport of charged organic compounds such as one or more of prostaglandins, thromboxanes, hexoses, disaccharides, hormones (e.g. insulin), peptides, neurotransmitters, cytokines, chemokines, and the like. Accordingly, in one aspect, the present invention provides isolated nucleic acid molecules encoding a polypeptide of the invention or a biologically active portion thereof. The present invention also provides nucleic acid molecules which are suitable as primers or hybridization probes for the detection of nucleic acids encoding a polypeptide of the invention.

The invention also features nucleic acid molecules which are at least 40% (or 50%, 60%, 70%, 80%, 90%, 95%, or 98%) identical to the nucleotide sequence of any of SEQ ID NOs: 1, 2, 4, 5, and 6, or a complement thereof

The invention features nucleic acid molecules which include a fragment of at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or 5000, 10000, 20000, 40000, or 80000 or more) consecutive nucleotide residues of any of SEQ ID NOs: 1, 2, 4, 5, and 6, or a complement thereof.

The invention also features nucleic acid molecules which include a nucleotide sequence encoding a protein having an amino acid sequence that is at least 50% (or 60%, 70%, 80%, 90%, 95%, or 98%) identical to the amino acid sequence of either of SEQ ID NOs: 3 and 7, or a complement thereof.

In preferred embodiments, the nucleic acid molecules have the nucleotide sequence of any of SEQ ID NOs: 1, 2, 4, 5, and 6.

Also within the invention are nucleic acid molecules which encode a fragment of a polypeptide having the amino acid sequence of either of SEQ ID NOs: 3 and 7, the fragment including at least 8 (10, 15, 20, 25, 30, 40, 50, 75, 100, 125, 150, or 200) consecutive amino acids of either of SEQ ID NOs: 3 and 7.

The invention includes nucleic acid molecules which encode a naturally occurring allelic variant of a polypeptide comprising the amino acid sequence of either of SEQ ID NOs: 3 and 7, wherein the nucleic acid molecule hybridizes under stringent conditions to a nucleic acid molecule having a nucleic acid sequence encoding any of SEQ ID NOs: 1, 2, 4, 5, and 6, or a complement thereof.

Also within the invention are isolated polypeptides or proteins having an amino acid sequence that is at least about 50%, preferably 60%, 75%, 90%, 95%, or 98% identical to the amino acid sequence of either of SEQ ID NOs: 3 and 7.

Also within the invention are isolated polypeptides or proteins which are encoded by a nucleic acid molecule having a nucleotide sequence that is at least about 40%, preferably 50%, 75%, 85%, or 95% identical to the nucleic acid sequence encoding either of SEQ ID NOs: 3 and 7, and isolated polypeptides or proteins which are encoded by a nucleic acid molecule consisting of the nucleotide sequence which hybridizes under stringent hybridization conditions to a nucleic acid molecule having the nucleotide sequence of any of SEQ ID NOs: 1, 2, 4, 5, and 6.

Also within the invention are polypeptides which are naturally occurring allelic variants of a polypeptide that includes the amino acid sequence of either of SEQ ID NOs: 3 and 7, wherein the polypeptide is encoded by a nucleic acid molecule which hybridizes under stringent conditions to a nucleic acid molecule having the nucleotide sequence of any of SEQ ID NOs: 1, 2, 4, 5, and 6, or a complement thereof.

The invention also features nucleic acid molecules that hybridize under stringent conditions to a nucleic acid molecule having the nucleotide sequence of any of SEQ ID NOs: 1, 2, 4, 5, and 6, or a complement thereof. In other embodiments, the nucleic acid molecules are at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, 5000, 10000, 20000, 40000, or 80000 or more) nucleotides in length and hybridize under stringent conditions to a nucleic acid molecule having the nucleotide sequence of any of SEQ ID NOs: 1, 2, 4, 5, and 6, or a complement thereof In some embodiments, the isolated nucleic acid molecules encode a cytoplasmic, transmembrane, extracellular, or other domain of a polypeptide of the invention. In other embodiments, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention. In another embodiment, the invention provides isolated host cells, e.g., mammalian and non-mammalian cells, containing such a vector or a nucleic acid of the invention. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention. Preferred proteins and polypeptides possess at least one biological activity possessed by the corresponding naturally-occurring human polypeptide. An activity, a biological activity, and a functional activity of a polypeptide of the invention refers to an activity exerted by a protein or polypeptide of the invention on a responsive cell as determined in vivo, or in vitro, according to standard techniques. Such activities can be a direct activity, such as an association with or an enzymatic activity on a second protein or an indirect activity, such as a cellular processes mediated by interaction of the protein with a second protein.

By way of example, proteins 65h2 and 593, compounds which modulate their activity, expression, or both, and compounds (e.g. antibodies) which bind with 65h2 or 593 (collectively “65h2-related molecules” and “593-related molecules) exhibit the ability to affect growth, proliferation, survival, differentiation, and activity of tissues in which they are normally expressed and tissues upon which they normally act. Such tissues include, by way of example, epithelial tissues, neuronal tissues, eye tissues, ova, spermatozoa, uterine tissues, liver tissue, lung tissue, blood tissues, cardiovascular tissues and the like. Thus, 65h2- and 593-related molecules can be used to prognosticate, prevent, diagnose, or treat disorders relating to inappropriate transmembrane transport of charged organic compounds such as prostaglandins and thromboxanes. Exemplary disorders for which 65h2- and 593-related molecules are useful include diabetes, nutritional disorders (e.g. vitamin deficiencies, and malnutrition), metabolic disorders (e.g. obesity, porphyrias, hyper- and hypolipoproteinemia, lipidoses, and water, electrolyte, mineral, and acid/base imbalances), neural transmission disorders (e.g. inappropriate pain, dementia, multiple sclerosis, nerve root disorders, Alzheimer's disease, Parkinson's disease, depression, physical and psychological substance addiction, sexual dysfunction, schizophrenic disorders, delusional disorders, mood disorders, and sleep disorders), stroke, regeneration of chronically or traumatically damaged neuronal structures (including nerve, brain, and spinal cord), developmental neuronal disorders (e.g. spina bifida), neuronal cancers (e.g. gliomas, astrocytomas, ependymomas, pituitary adenomas, and the like), peripheral nerve deficit, coronary insufficiency, angina, glaucoma, ovum fertilization, sperm motility, pregnancy-related disorders (e.g. miscarriage), gastric disorders such as peptic ulcer, inappropriate intestinal fluid secretion, liver damage, liver fibrosis, inappropriate pain, glomerular filtration disorders, body temperature maintenance disorders such as fever, airway resistance disorders such as asthma, chronic obstructive pulmonary disorder, blood pressure modulation disorders such as hypertension and shock, inflammation, platelet aggregation, abnormal blood coagulation, atherosclerosis, arteriosclerosis, coronary artery disease, and the like.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common domain and/or common functional activity. For example, amino acid or nucleotide sequences which contain a common domain having about 65% identity, preferably 75% identity, more preferably 85%, 95%, or 98% identity are defined herein as sufficiently identical.

In one embodiment, the isolated polypeptide of the invention lacks both a transmembrane and a cytoplasmic domain. In another embodiment, the polypeptide lacks both a transmembrane domain and a cytoplasmic domain and is soluble under physiological conditions.

The polypeptides of the present invention, or biologically active portions thereof, can be operably linked to a heterologous amino acid sequence to form fusion proteins. The invention further features antibody substances that specifically bind a polypeptide of the invention such as monoclonal or polyclonal antibodies, antibody fragments, single-chain antibodies, and the like. In addition, the polypeptides of the invention or biologically active portions thereof can be incorporated into pharmaceutical compositions, which optionally include pharmaceutically acceptable carriers. These antibody substances can be made, for example, by providing the polypeptide of the invention to an immunocompetent vertebrate and thereafter harvesting blood or serum from the vertebrate.

The invention is also based on discovery that a cDNA clone previously sequenced by others (who did not know the function of the encoded protein) encodes a prostaglandin/thromboxane transmembrane transport protein designated KIAA0880. This protein and fragments, derivatives, and variants thereof (collectively, “KIAA0880-related polypeptides) exhibit the physiological characteristics and activities described above.

In another aspect, the present invention provides methods for detecting the presence of the activity or expression of a polypeptide of the invention, or of a KIAA0880-related polypeptide, in a biological sample by contacting the biological sample with an agent capable of detecting an indicator of activity such that the presence of activity is detected in the biological sample.

In another aspect, the invention provides methods for modulating activity of a polypeptide of the invention, or activity of a KIAA0880-related polypeptide, the methods comprising contacting a cell with an agent that modulates (inhibits or enhances) the activity or expression of the polypeptide, such that activity or expression in the cell is modulated. In one embodiment, the agent is an antibody that specifically binds with the polypeptide of the invention or to the KIAA0880-related polypeptide.

In another embodiment, the agent modulates expression of a polypeptide of the invention, or of a KIAA0880-related polypeptide, by modulating transcription, splicing, or translation of an mRNA encoding the polypeptide of the invention or the KIAA0880-related polypeptide. In yet another embodiment, the agent is a nucleic acid molecule having a nucleotide sequence that is antisense with respect to the coding strand of an mRNA encoding a polypeptide of the invention or a KIAA0880-related polypeptide.

The present invention also provides methods to treat a subject having a disorder characterized by aberrant activity of a polypeptide of the invention, aberrant expression of a nucleic acid of the invention, aberrant activity of a KIAA0880-related polypeptide, or aberrant expression of a nucleic acid encoding a KIAA0880-related polypeptide, by administering an agent which is a modulator of the activity of the polypeptide or a modulator of expression of the nucleic acid to the subject. In one embodiment, the modulator is a protein of the invention or a KIAA0880-related polypeptide. In another embodiment, the modulator is a nucleic acid of the invention or a nucleic acid encoding a KIAA0880-related polypeptide. In other embodiments, the modulator is a peptide, peptidomimetic, or other small molecule.

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention. In addition, the invention provides diagnostic assays for identifying the presence or absence of a genetic lesion or mutation characterized by at least one of: (i) aberrant modification or mutation of a gene encoding a KIAA0880-related polypeptide, (ii) mis-regulation of a gene encoding a KIAA0880-related polypeptide, and (iii) aberrant post-translational modification of a KIAA0880-related polypeptide wherein a wild-type form of the gene encodes a polypeptide having the activity of the KIAA0880-related polypeptide.

In another aspect, the invention provides a method for identifying a compound that binds with or modulates the activity of a polypeptide of the invention or a KIAA0880-related polypeptide. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention, of a KIAA0880-related polypeptide, or of a nucleic acid encoding a KIAA0880-related polypeptide, by measuring the expression of the polypeptide or nucleic acid in the presence and absence of the compound.

In yet another aspect, the invention includes a method of treating a patient afflicted with a disorder associated with aberrant activity or expression of a protein selected from the group consisting of 65h2, 593, and KIAA0880. The method comprises administering to the patient a compound (e.g. a nucleic acid, polypeptide, small molecule, antibody, or the like) which modulates the activity of the protein in an amount effective to modulate the activity of the protein in the patient. Following administration of the compound, at least one symptom of the disorder is alleviated. In an alternate method of treating a patient afflicted with a disorder associated with aberrant activity or expression of a protein selected from the group consisting of 65h2, 593, and KIAA0880, the method comprises administering to the patient, in an amount effective to modulate the activity of the protein in the patient, a compound selected from the group consisting of

i) the protein;

ii) a variant of the protein;

iii) a nucleic acid encoding the protein; and

iv) an antisense nucleic acid which is capable of annealing with either of an mRNA encoding the protein and a portion of a genomic DNA encoding the protein.

Following administration of the compound, at least one symptom of the disorder is alleviated.

In still another aspect of the invention, the invention relates to a method of diagnosing a disorder associated with aberrant expression of a protein selected from the group consisting of 65h2, 593, and KIAA0880 in a patient. This method comprises assessing the level of expression of the gene encoding the protein (e.g. by assessing the quantity of a corresponding RNA, the quantity of a corresponding protein, or the activity of a corresponding protein) in the patient and comparing the level of expression of the gene with the normal level of expression of the gene in a human not afflicted with the disorder. A difference between the level of expression of the gene in the patient and the normal level is an indication that the patient is afflicted with the disorder.

Other features and advantages of the invention will be apparent from the following detailed description and claims.

### BRIEF DESCRIPTION OF THE DRAWINGS

FIG. 1 comprises FIGS. 1A through 1O. The nucleotide sequence (SEQ ID NO: 1) of a cDNA encoding the human 65h2 protein described herein is listed in FIGS. 1A through 1E. The open reading frame (ORF; residues 42 to 1970; SEQ ID NO: 2) of the cDNA is indicated by nucleotide triplets, above which the amino acid sequence (SEQ ID NO: 3) of human 65h2 is listed. FIGS. 1F and 1G list the nucleotide sequence of the cDNA encoding human 65h2 protein (SEQ ID NO: 1). FIG. 1H lists the amino acid sequence of 65h2 protein (SEQ ID NO: 3). An alignment of the amino acid sequences of human 65h2 protein (“65h2”; SEQ ID NO: 3) and human prostaglandin transport protein (“HPT”; SEQ ID NO: 11) is shown in FIGS. 1I through 1K, wherein identical amino acid residues are indicated by “:” and similar amino acid residues are indicated by “.”. FIG. 1L, comprising FIGS. 1L(i) through 1L(xxxix), lists the genomic sequence encoding human 65h2 protein. FIG. 1M, comprising FIGS. 1M a) through 1M m), is a series of plots described herein. FIG. 1N is a hydrophilicity plot of human 65h2 protein, in which the locations of cysteine residues (“Cys”) and potential N-glycosylation sites (“Ngly”) are indicated by vertical bars and the predicted extracellular (“out”), intracellular (“ins”), or transmembrane (“TM”) locations of the protein backbone is indicated by a horizontal bar. FIG. 1O is an alignment of the Pfam consensus sequence (“C”; upper row) of the Pfam Sugar (or other) transport domain and amino acid sequence of residues 1 to 446 of 65h2 protein (“65h2”; i.e. residues 1 to 446 of SEQ ID NO: 3; lower row). In FIG. 1O, dots represent regions of low sequence complexity, letters in the center row indicate identical amino acid residues, and “+” indicates similar amino acid residues.

FIG. 2 comprises FIGS. 2A through 2H. The nucleotide sequence (SEQ ID NO: 5) of a cDNA encoding the human 593 protein described herein is listed in FIGS. 2A and 2B. The open reading frame (ORF; residues 1 to 1836 of SEQ ID NO: 5; SEQ ID NO: 6) is listed in FIGS. 2C and 2D. The amino acid sequence (SEQ ID NO: 7) of human 593 protein is listed in FIG. 2E. FIG. 2F, comprising FIG. 2F a) through FIG. 2F m), is a series of plots described herein. FIG. 2G is a hydrophilicity plot of human 593 protein. FIG. 2H is an alignment of the Pfam consensus sequence (“C”; upper row) of the Pfam Sugar (or other) transport domain and amino acid sequence of residues 2 to 490 of 593 protein (“593”; i.e. residues 2 to 490 of SEQ ID NO: 7; lower row). In FIG. 2H, dots represent regions of low sequence complexity, letters in the center row indicate identical amino acid residues, and “+” indicates similar amino acid residues.

FIG. 3 comprises FIGS. 3A through 3I. The nucleotide sequence (SEQ ID NO: 8) of a cDNA encoding the human protein designated KIAA0880 is listed in FIGS. 3A through 3D. The amino acid sequence of KIAA0880 is listed in FIG. 3E (SEQ ID NO: 9). An alignment of the amino acid sequences of human KIAA0880 protein (SEQ ID NO: 9) and human prostaglandin transport protein (“HPT”; SEQ ID NO: 11) is shown in FIGS. 3F through 3I. FIG. 3J is a hydrophilicity plot of human KIAA0880 protein.

FIG. 4, comprising FIGS. 4A through 4E, is an alignment of the amino acid sequences of human protein 65h2 (described herein; SEQ ID NO: 3), human prostaglandin transport protein (GenBank Accession no. Q92959; SEQ ID NO: 11), human OatP sodium-independent organic anion transporter protein (GenBank Accession no. P46721; SEQ ID NO: 10), human KIAA0880 protein (GenBank Accession no. 4240248 and described herein; SEQ ID NO: 9), and human protein 593 (described herein; SEQ ID NO: 7). In FIG. 4, asterisks indicate amino acid residues that are identical in all five sequences, colons (“:”) indicate amino acid residues at which only conservative amino acid differences exist among the five sequences, and periods (“.”) indicate amino acid residues at which at least semi-conservative amino acid differences exist among the five sequences.

FIG. 5 is a hydrophilicity plot of human prostaglandin transport protein (GenBank Accession no. Q92959).

### DETAILED DESCRIPTION OF THE INVENTION

The present invention is based, at least in part, on the discovery of human cDNA molecules which encode proteins which are herein designated 65h2 and 593. The invention is also based on the discovery that the protein encoded by a previously described (but otherwise non-characterized) human brain cDNA clone is, or is functionally analogous to, a prostaglandin and thromboxane transmembrane transport protein. These three proteins are integral membrane proteins that facilitate transmembrane transport of charged organic compounds such as one or more of prostaglandins, thromboxanes, hexoses, disaccharides, hormones (e.g. insulin), peptides, neurotransmitters, cytokines, chemokines, and the like. These three proteins are included in a single application for the sake of convenience. It is understood that the allowability or non-allowability of claims directed to one of these proteins has no bearing on the allowability of claims directed to the others. The characteristics of each of these proteins and the cDNAs encoding them are now described separately.

### Protein 65h2

A cDNA encoding at least a portion of human 65h2 protein was isolated from a library of human cDNA clones on the basis of homology to the amino terminal portion of the protein designated ‘human prostaglandin transporter’ (HPT) in the literature (U.S. Pat. No. 5,792,851; Lu et al. (1996) J. Clin. Invest. 98:1142-1149; Kanai et al. (1995) Science 268:866-869). Human protein 65h2 is predicted by structural analysis to be a transmembrane transporter protein having twelve transmembrane domains.

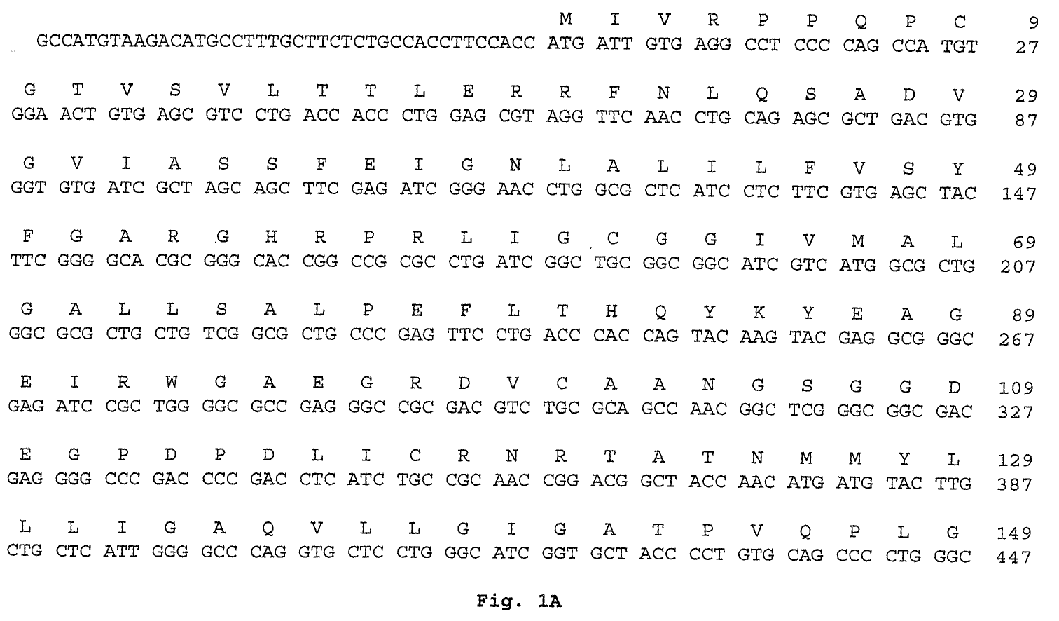

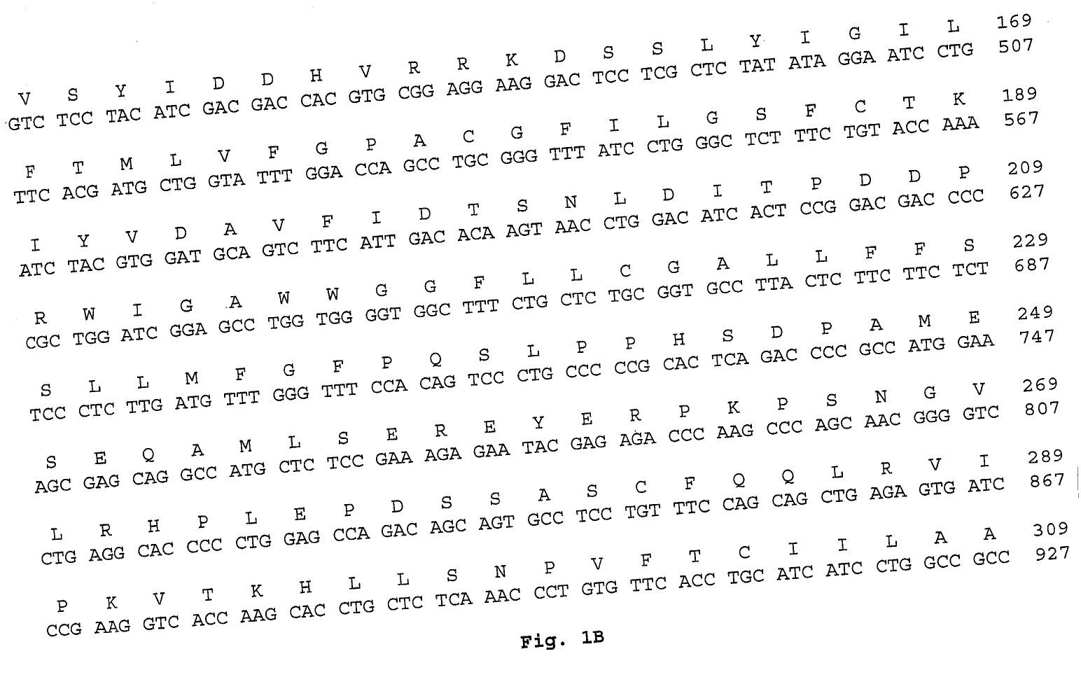

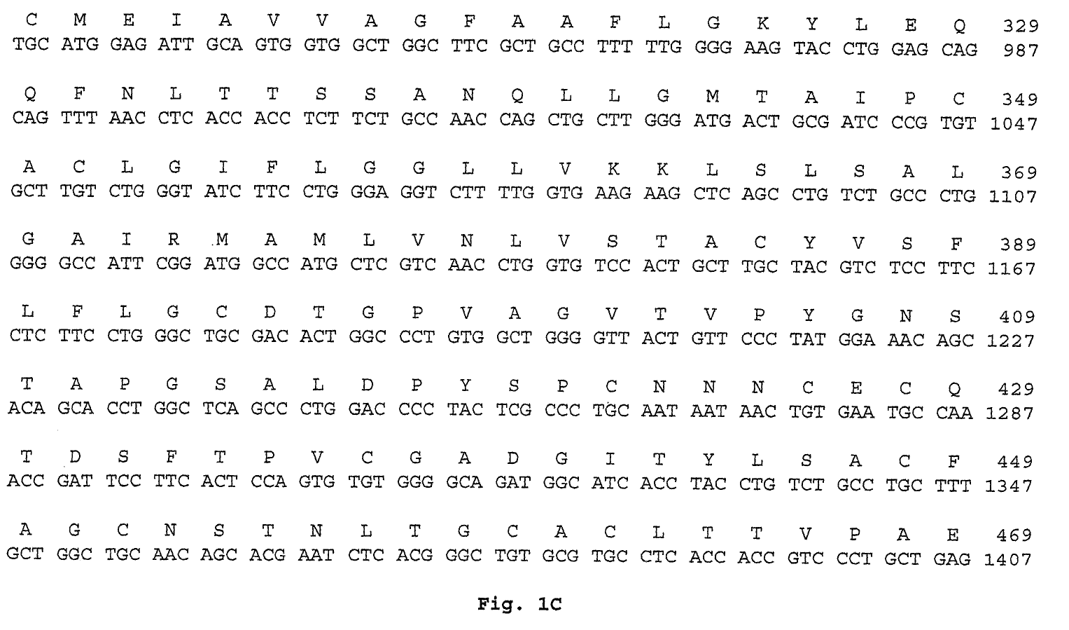

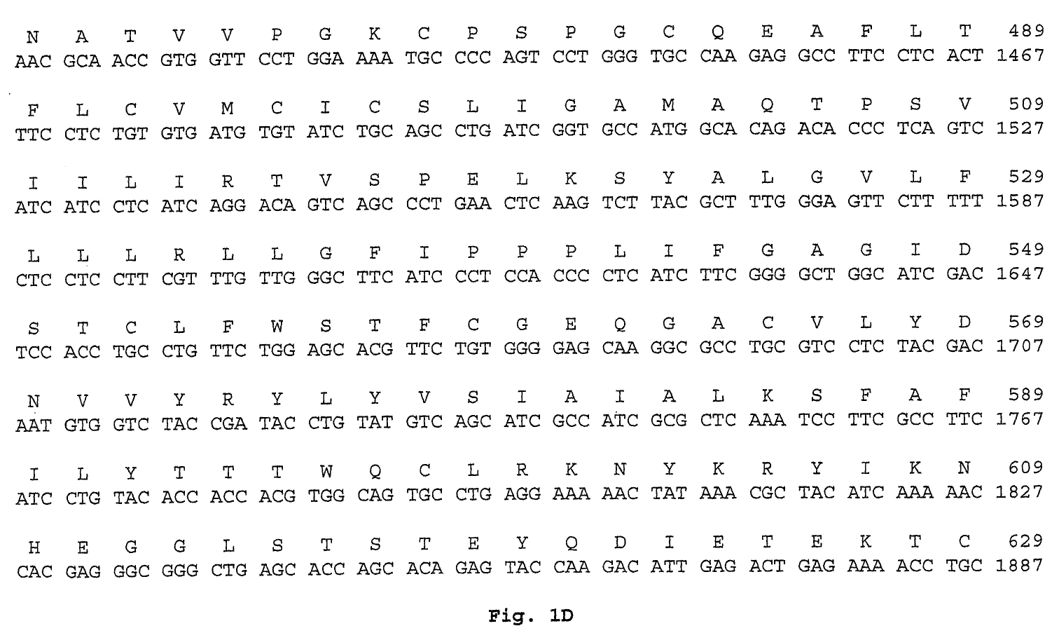

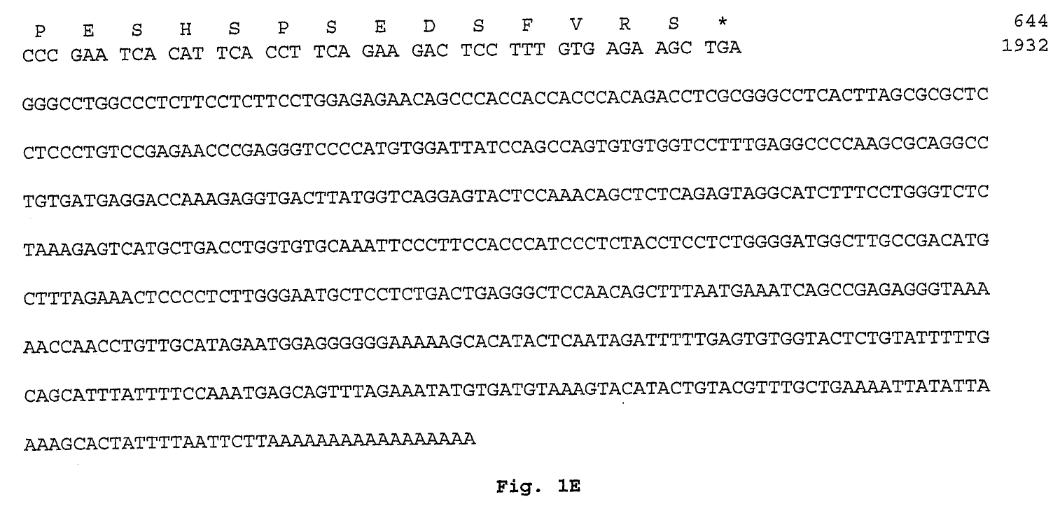

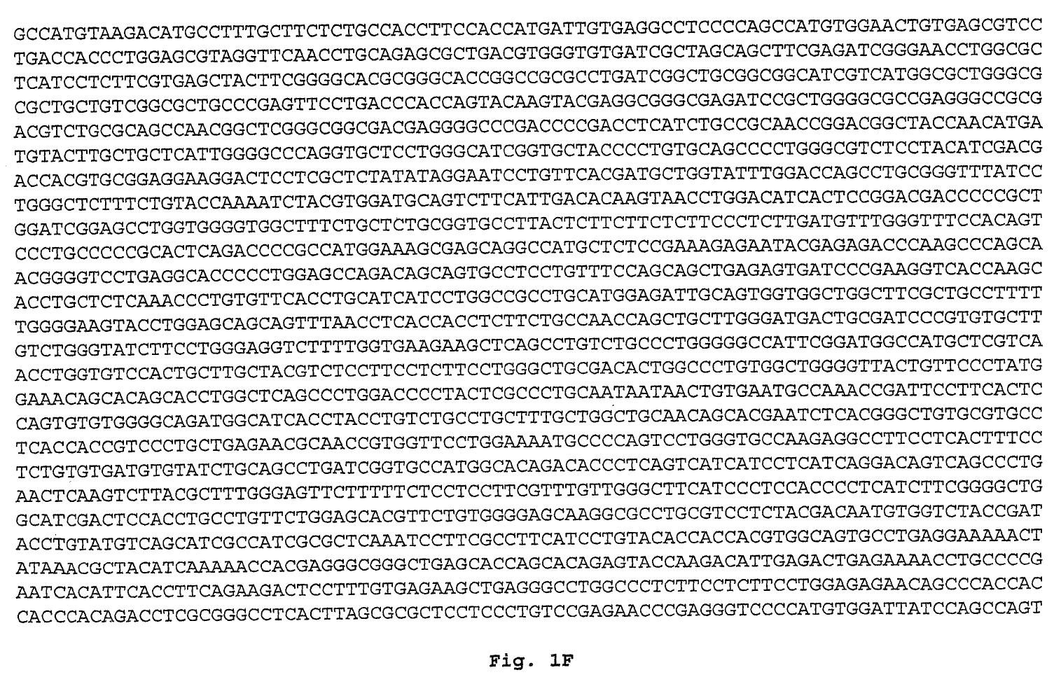

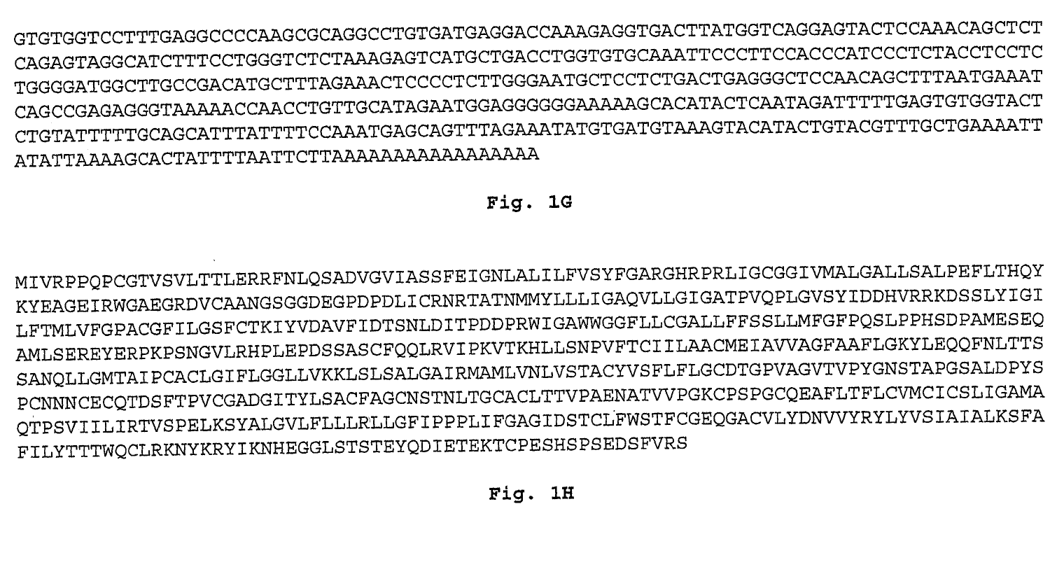

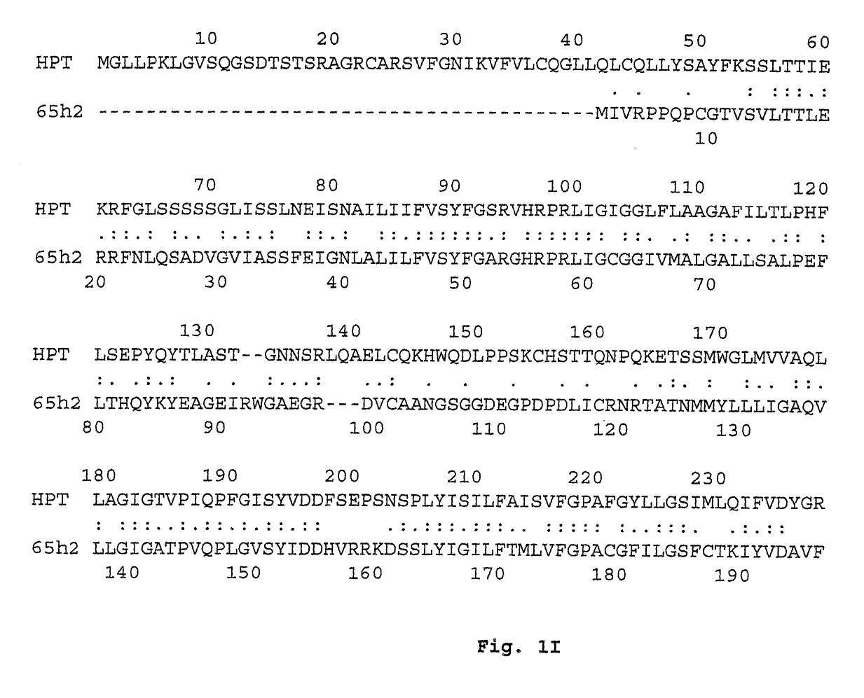

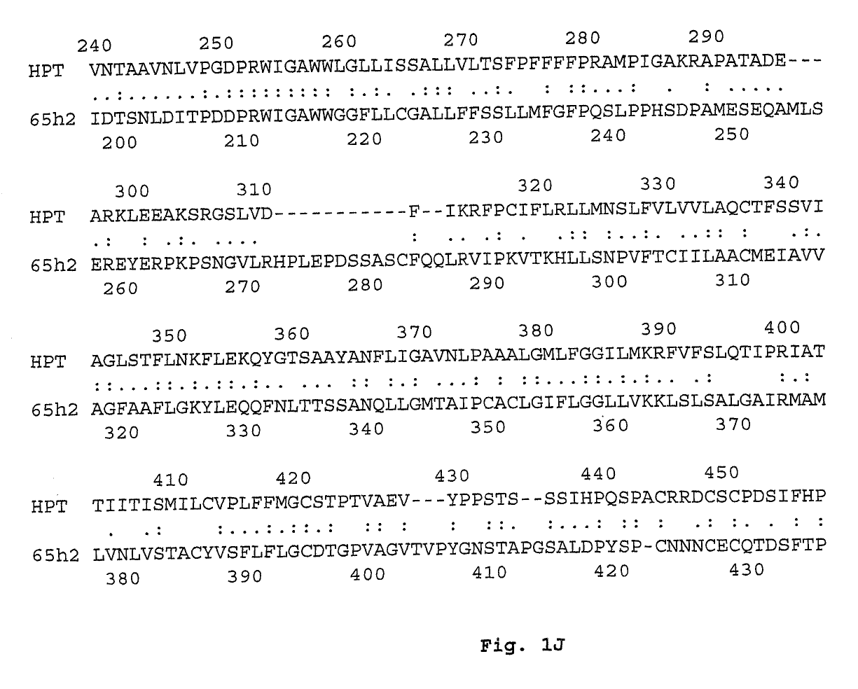

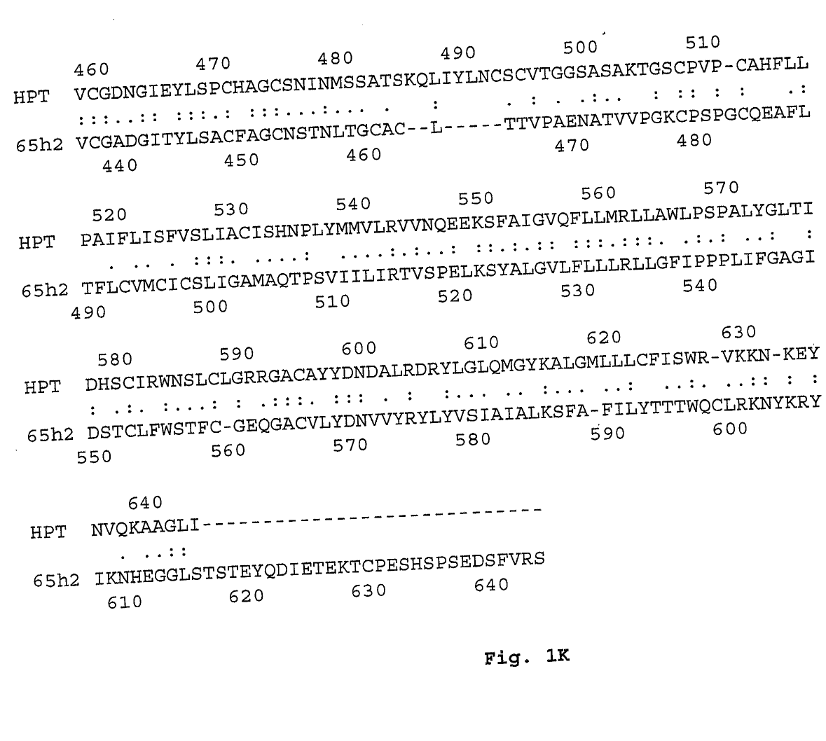

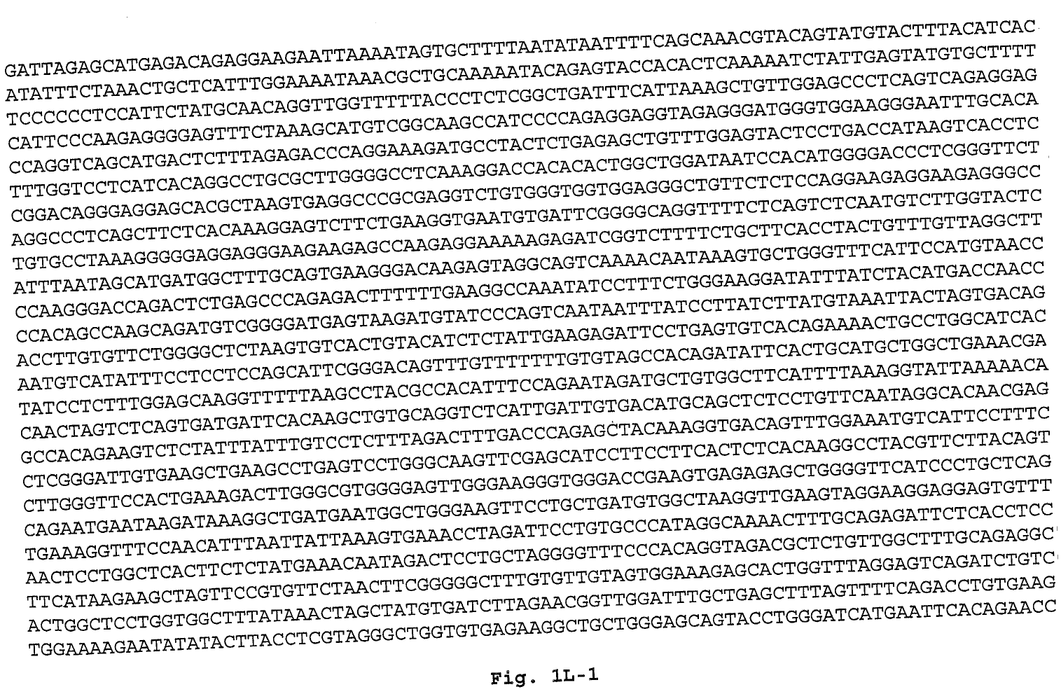

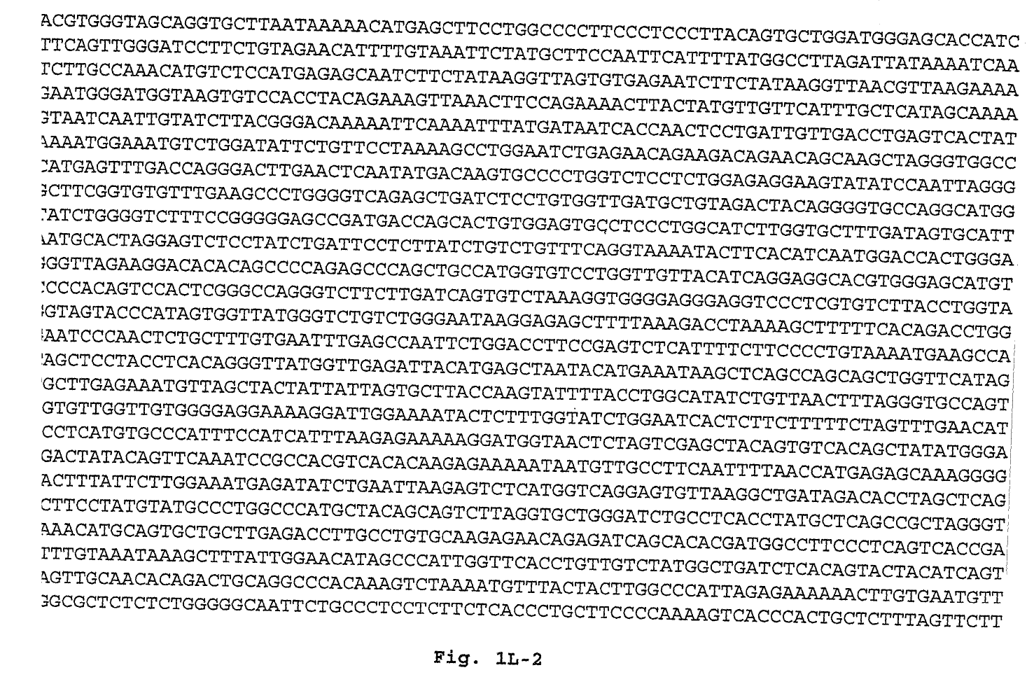

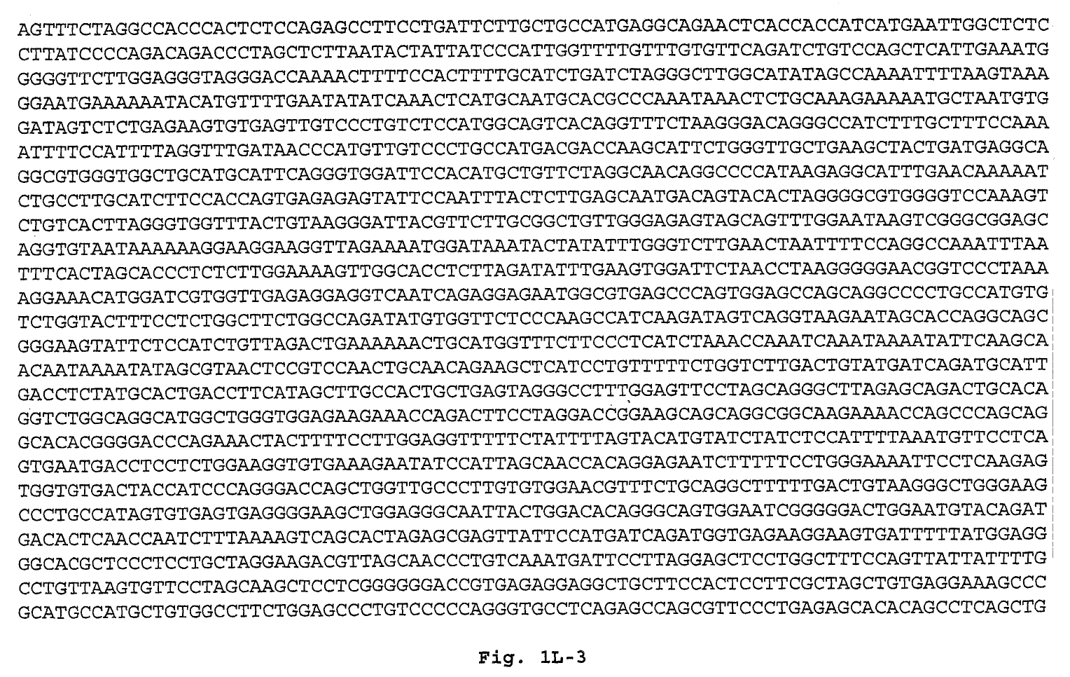

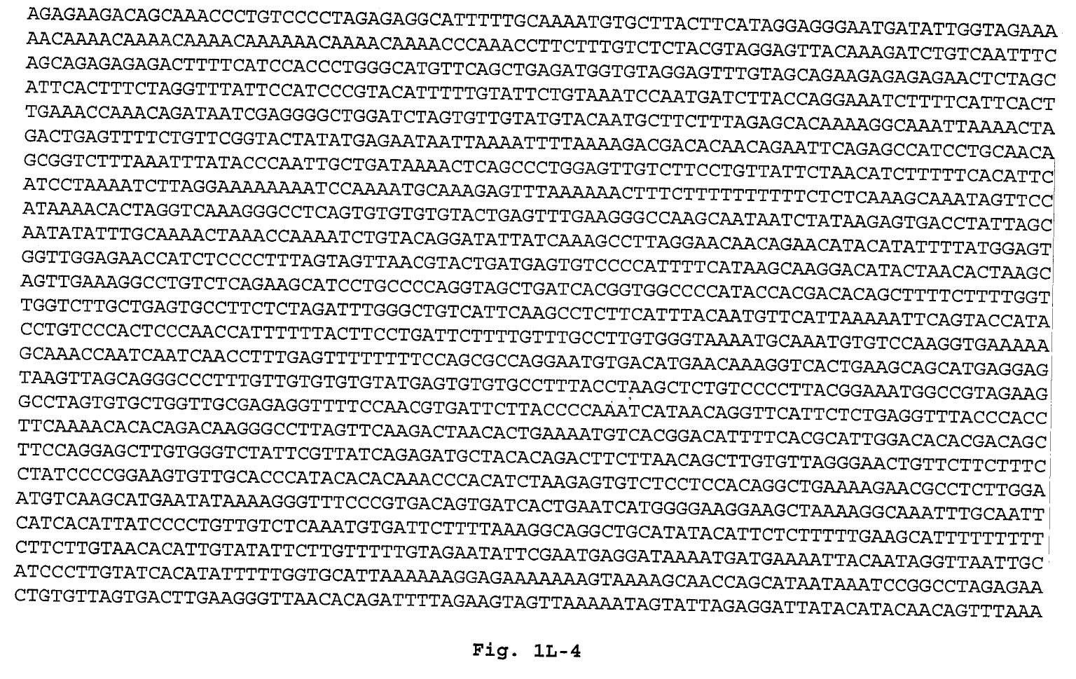

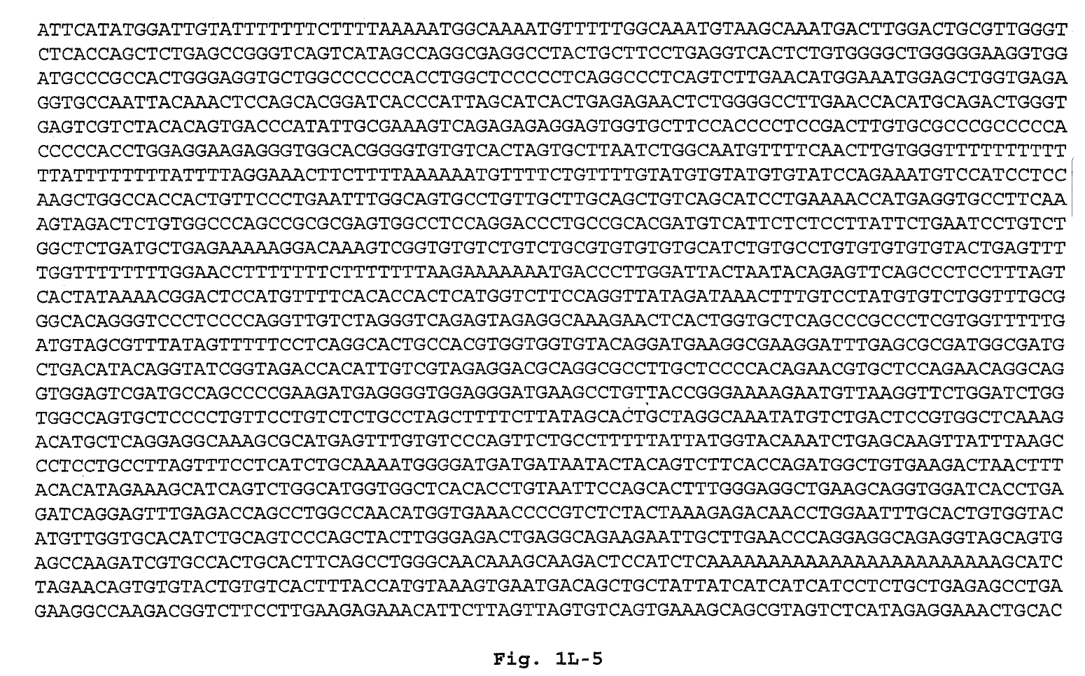

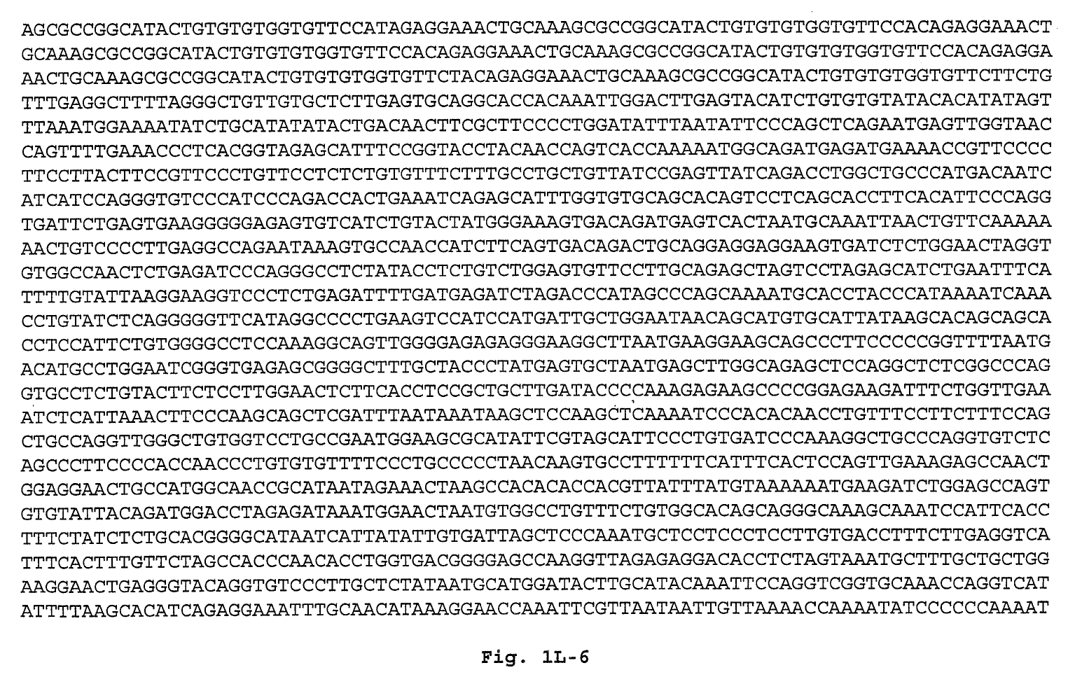

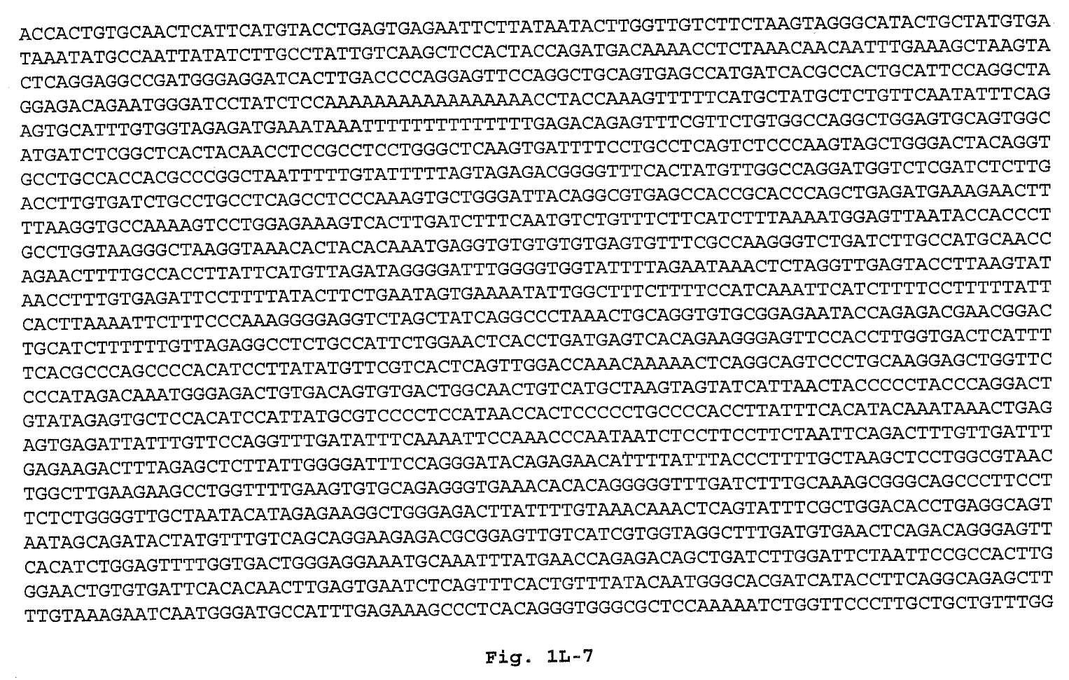

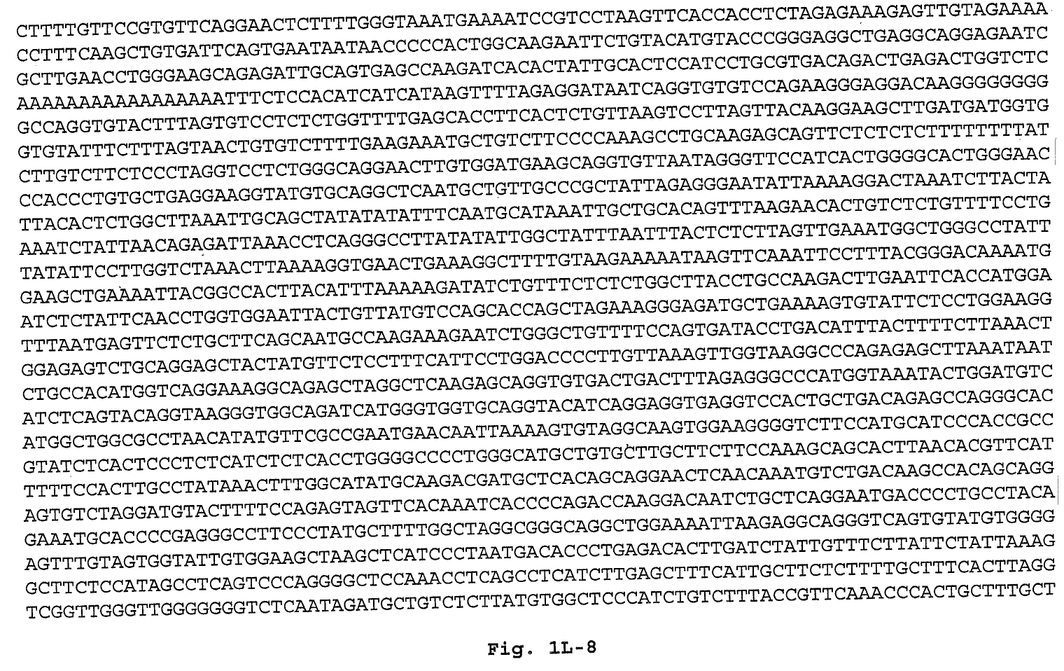

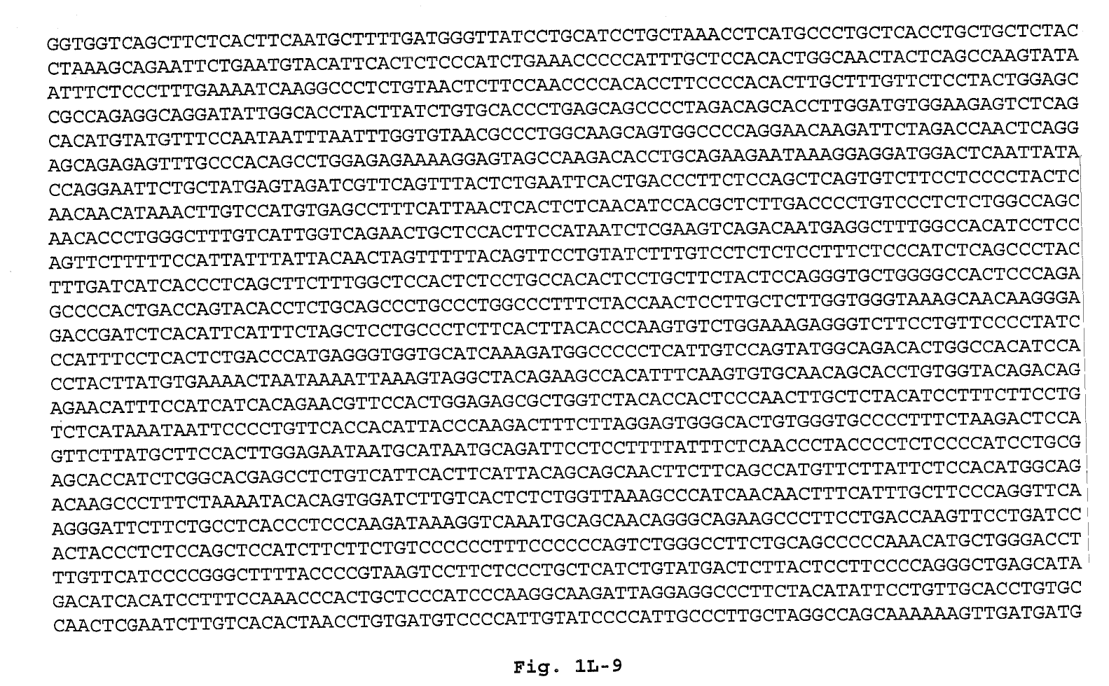

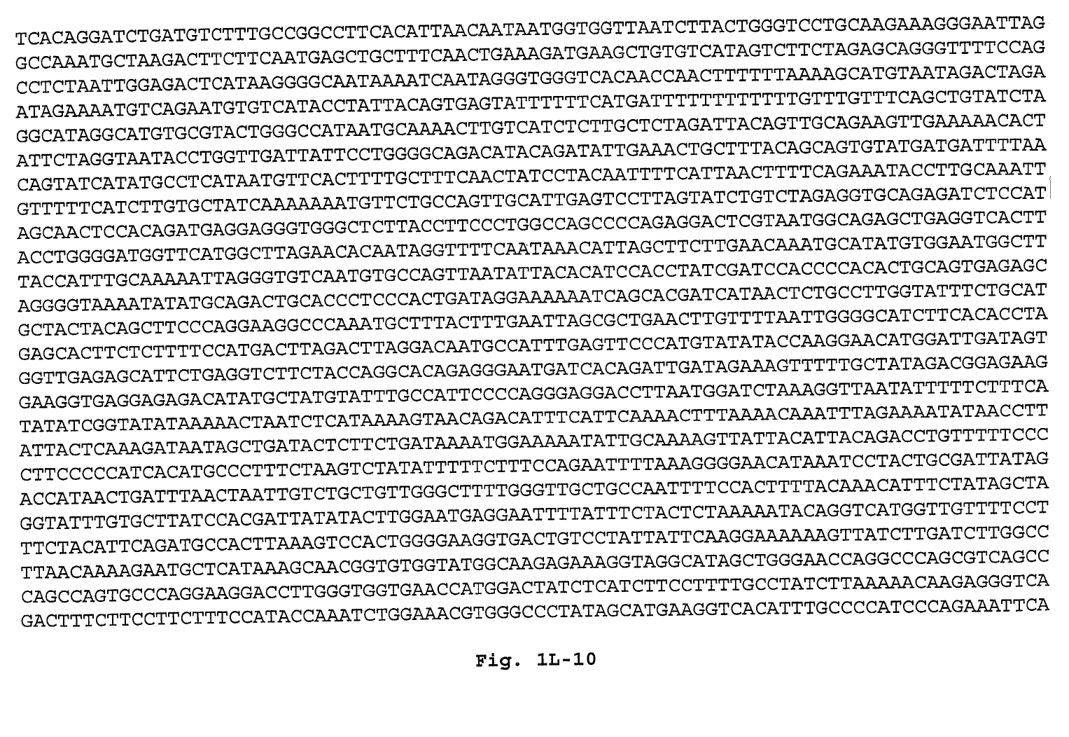

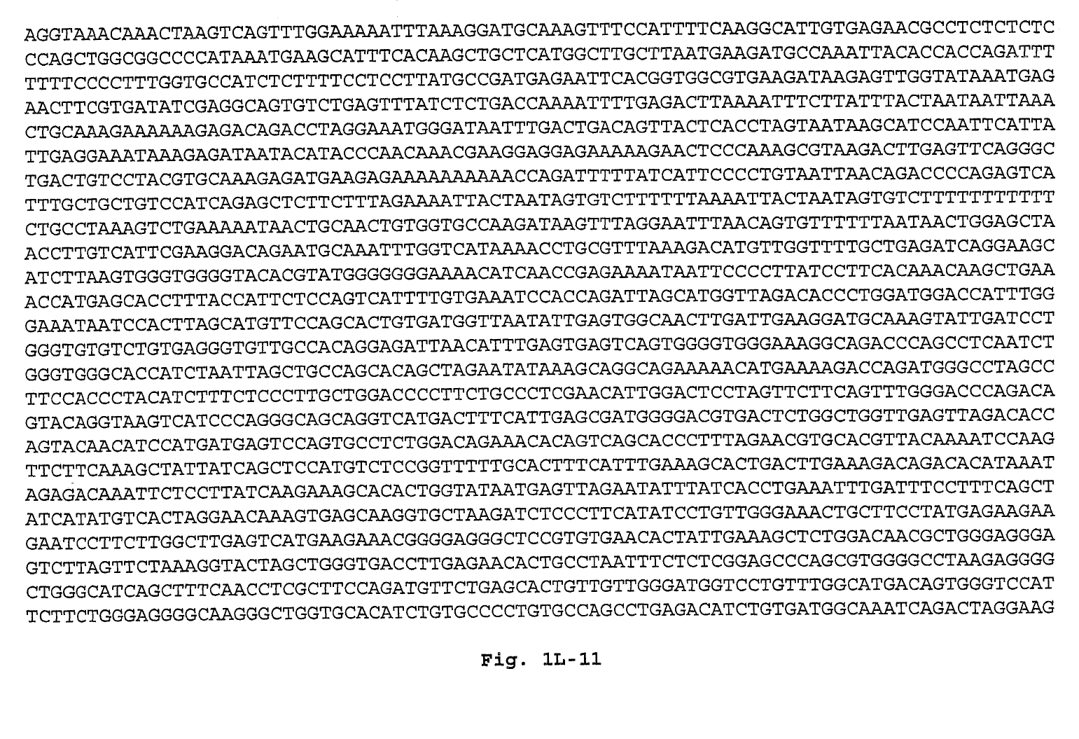

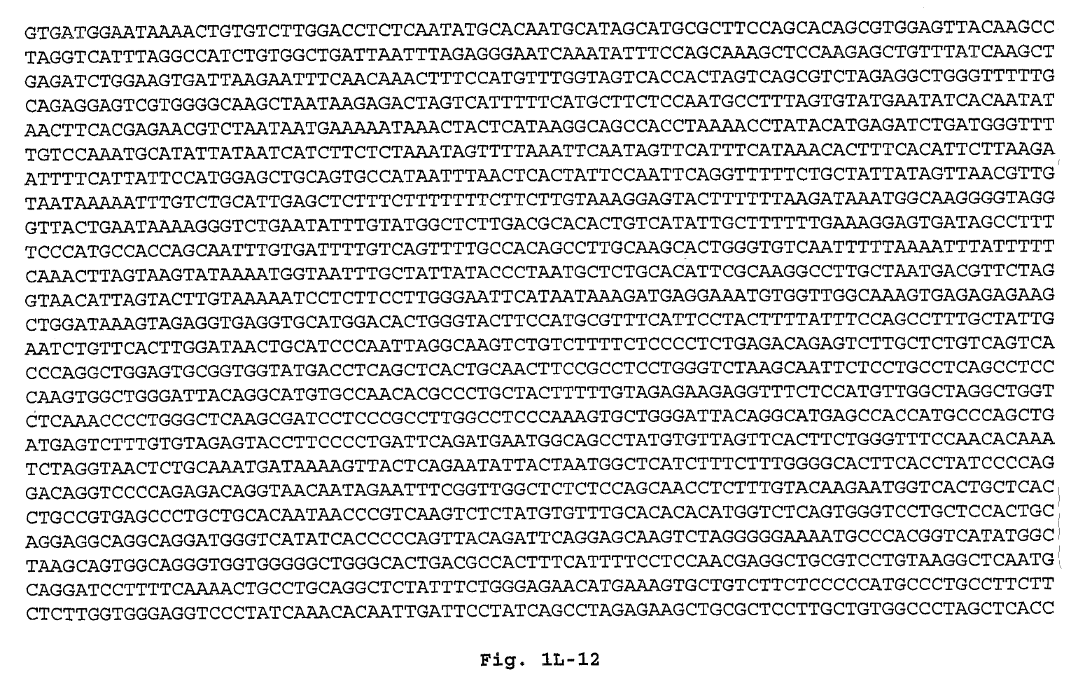

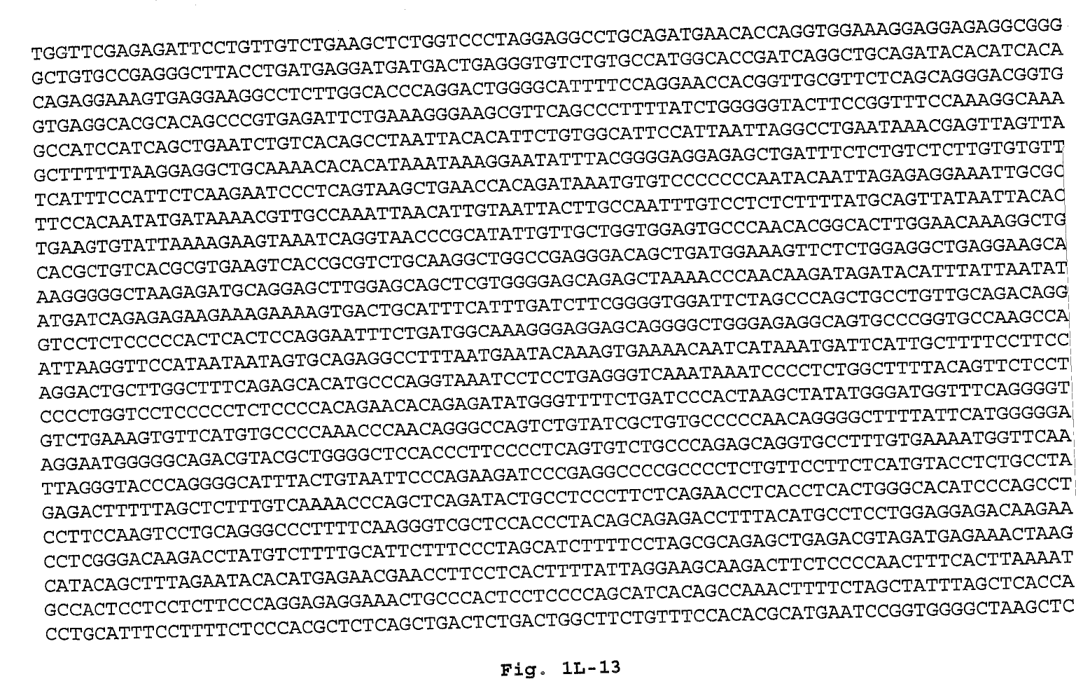

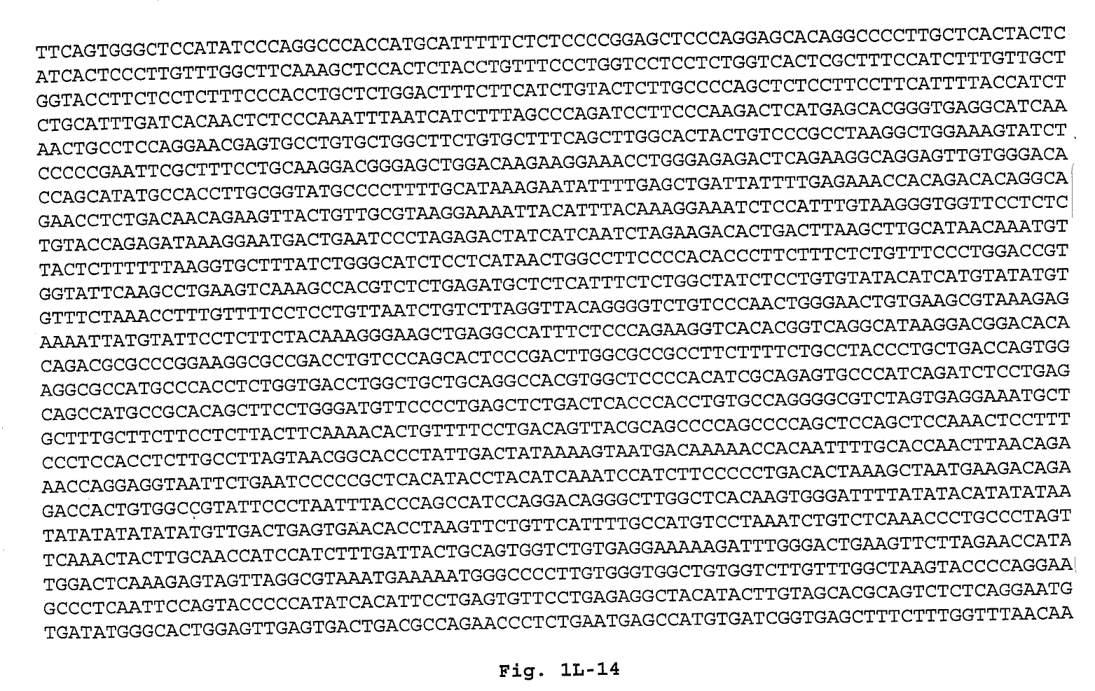

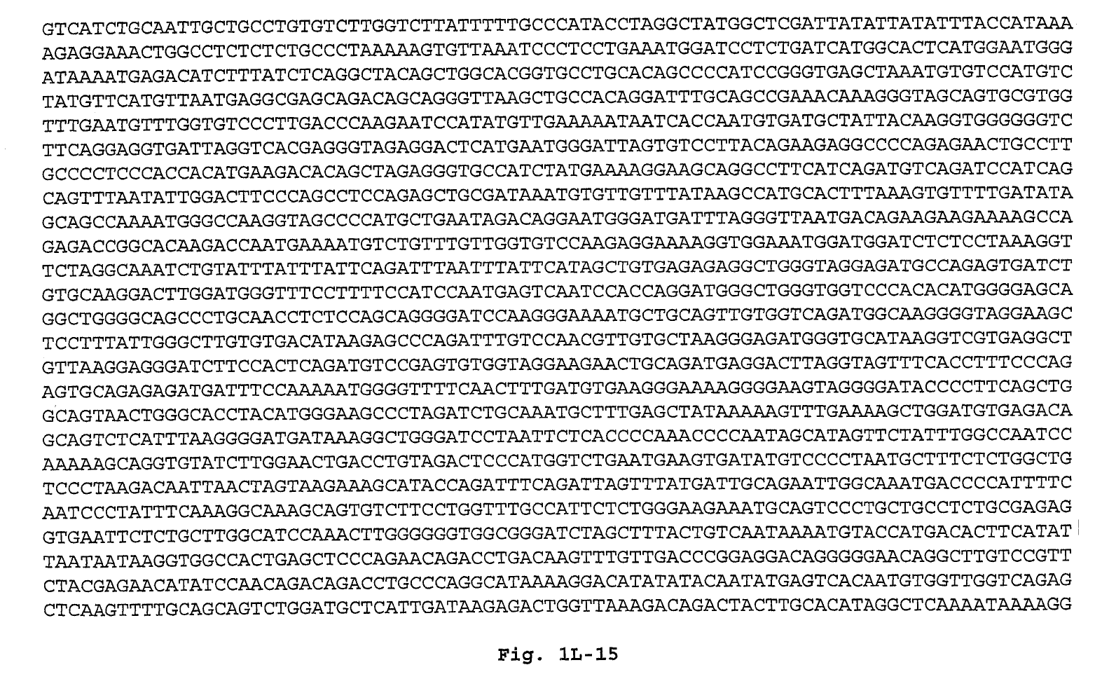

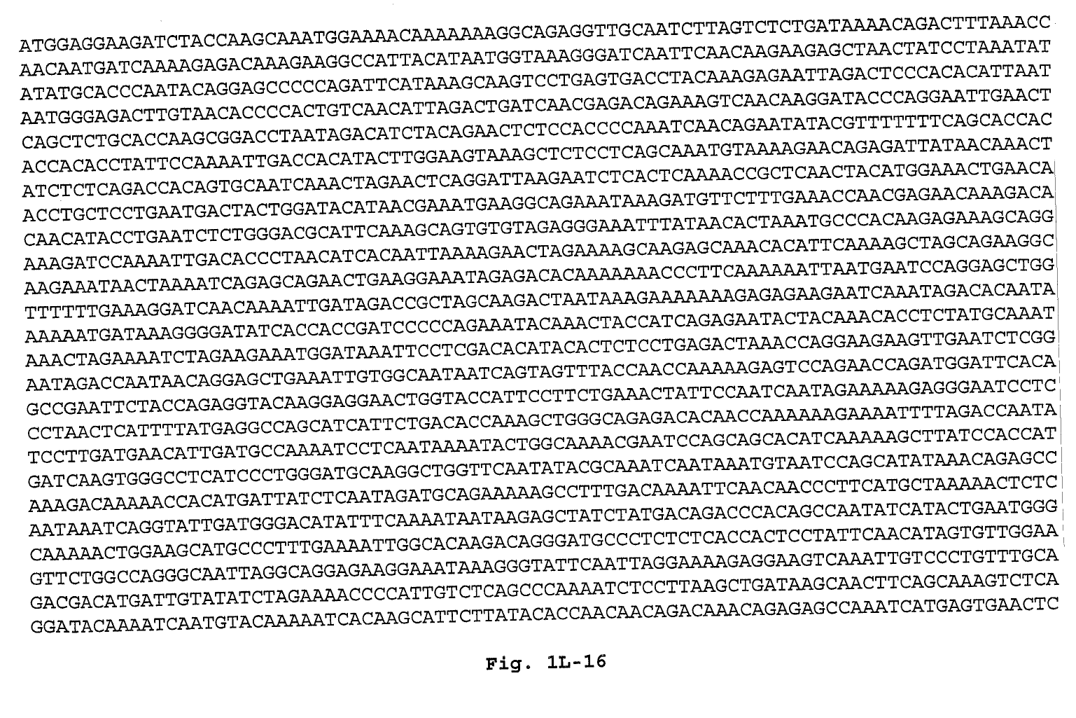

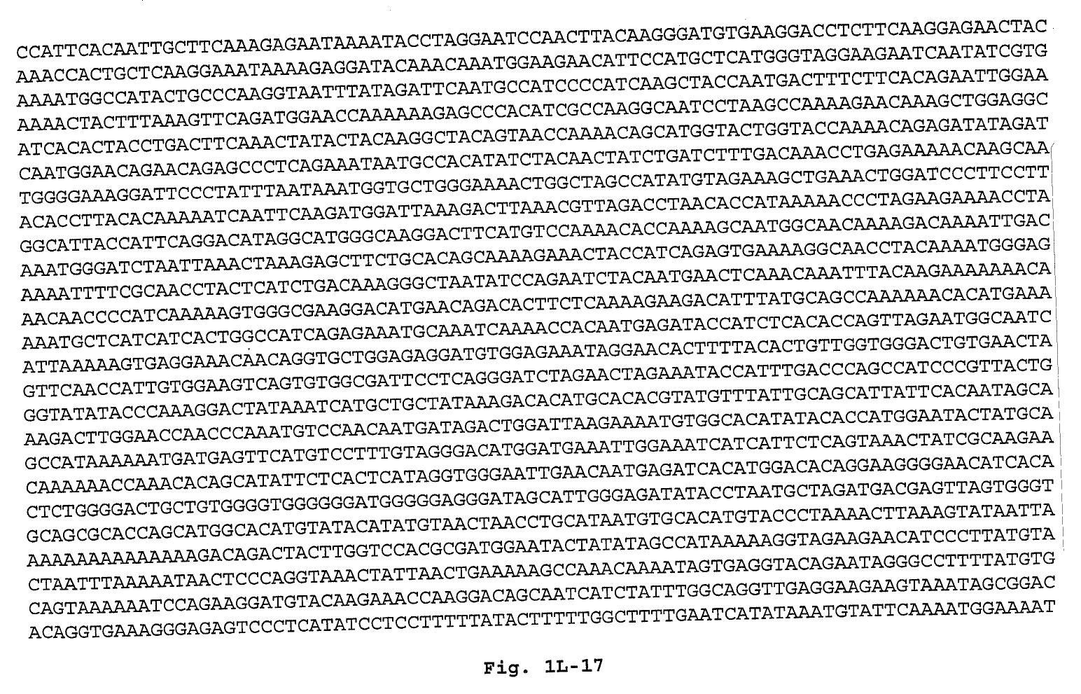

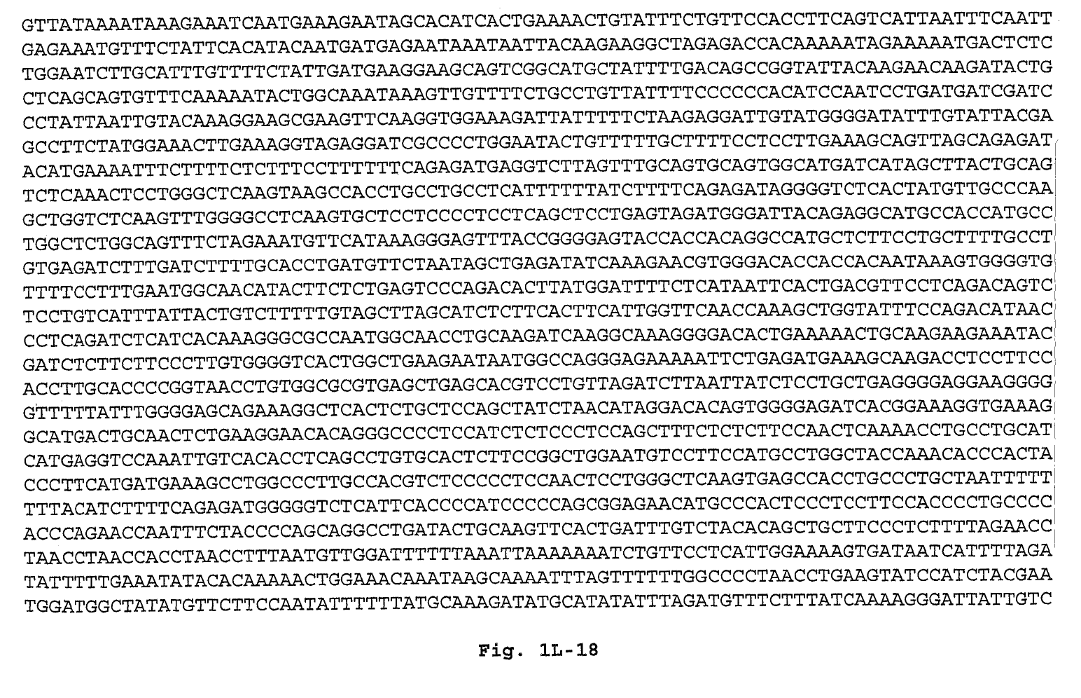

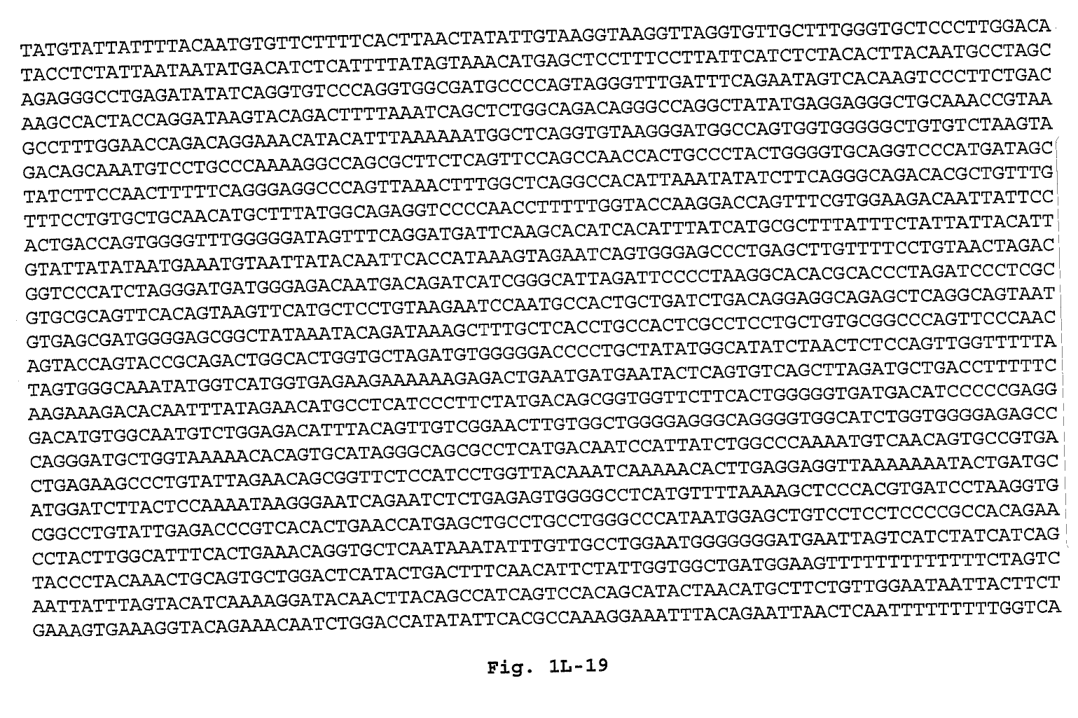

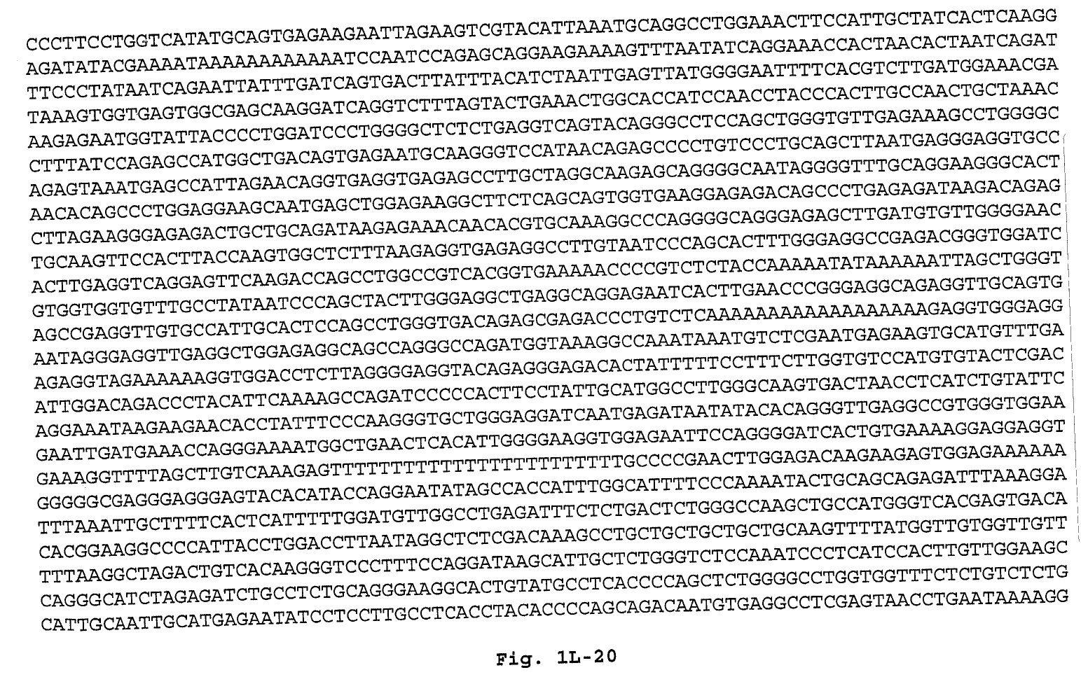

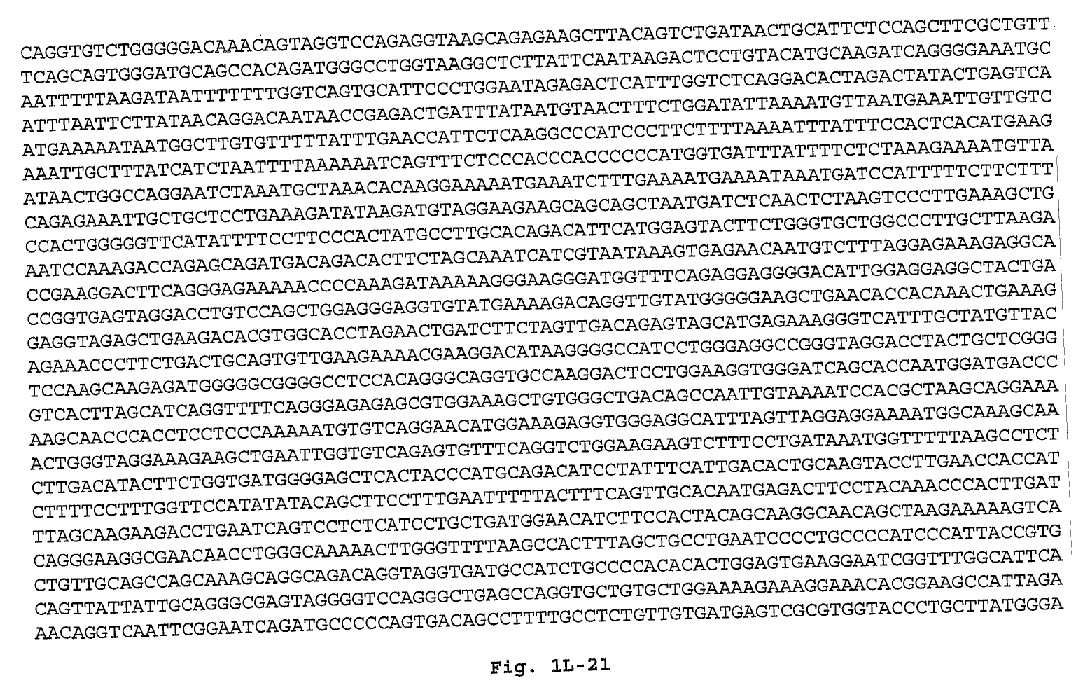

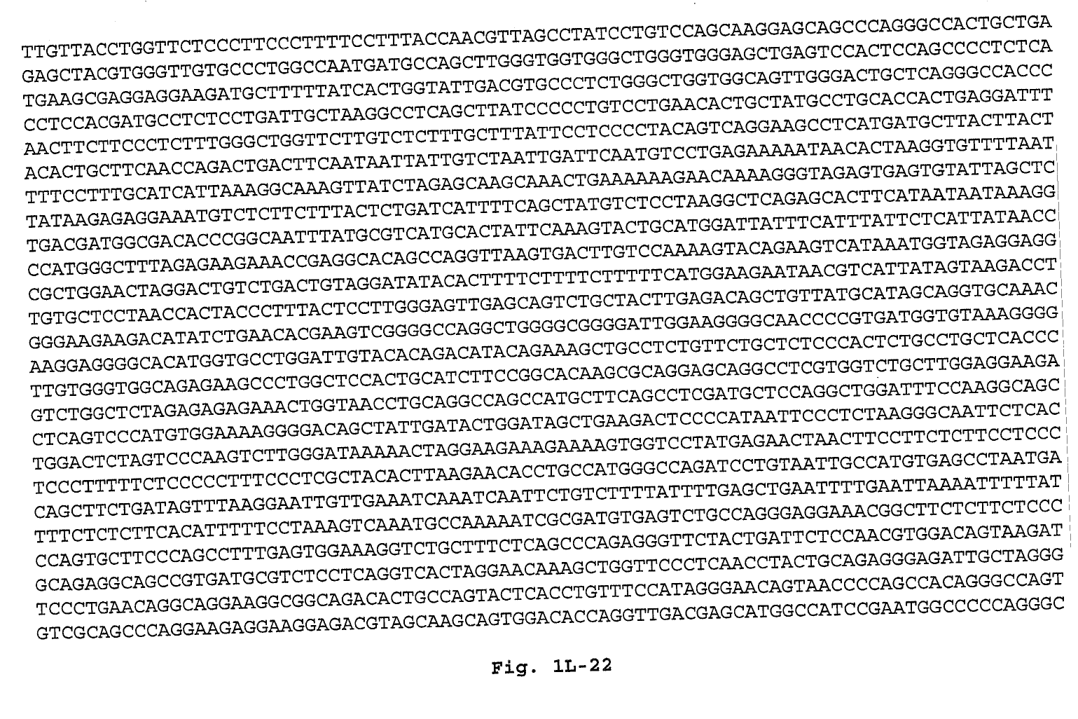

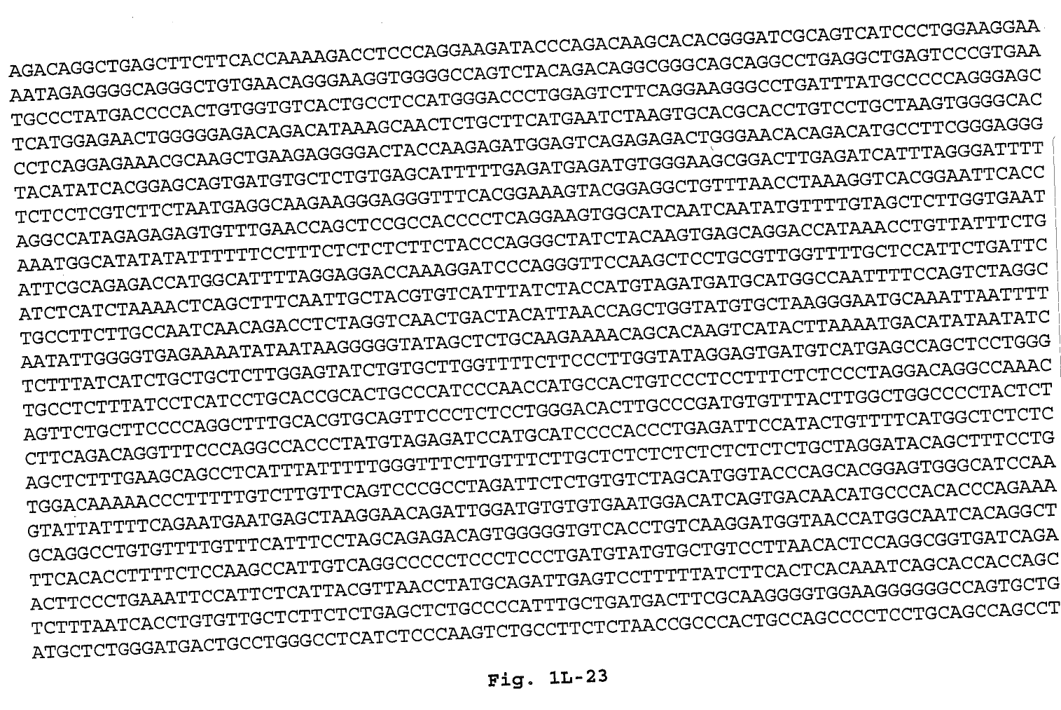

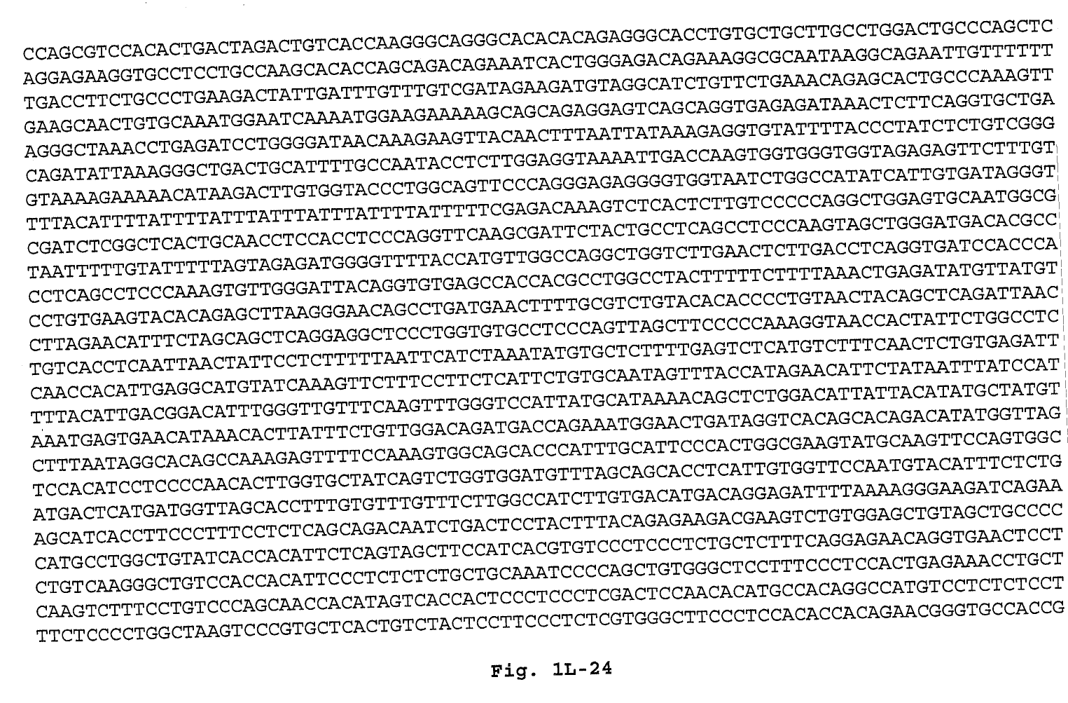

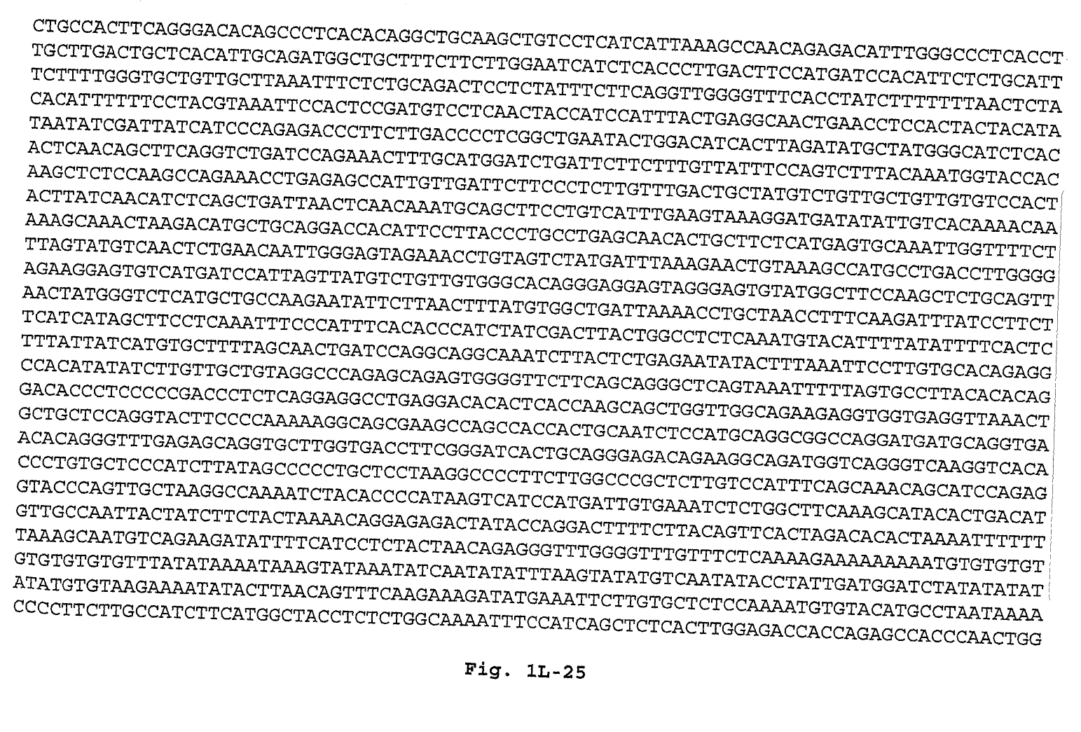

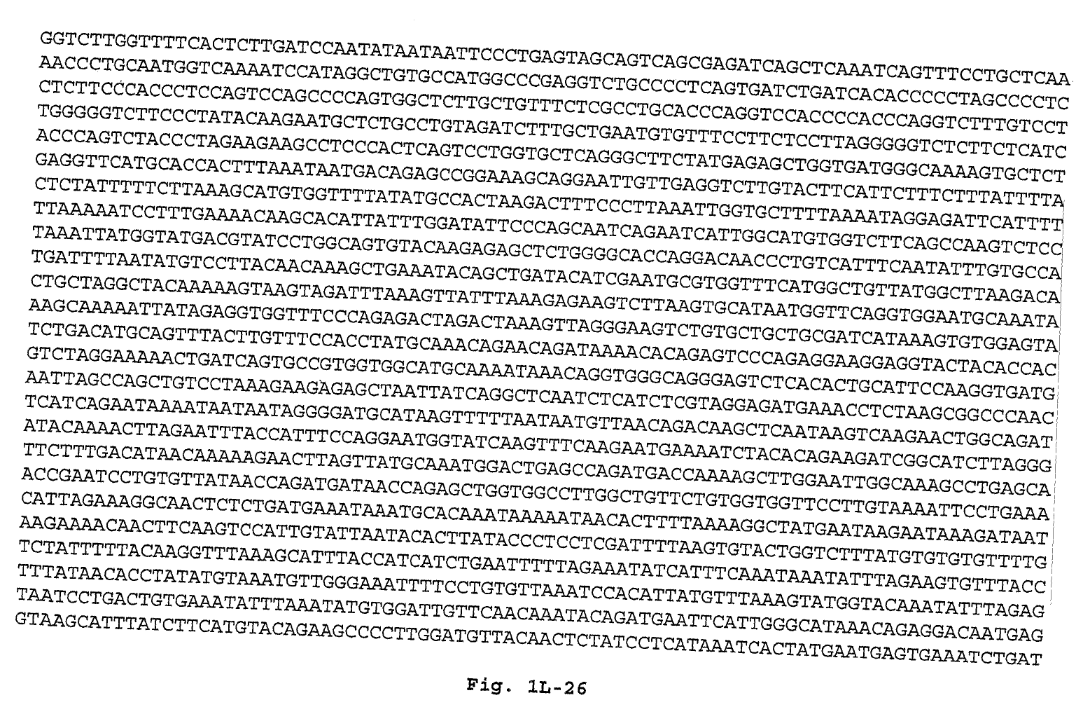

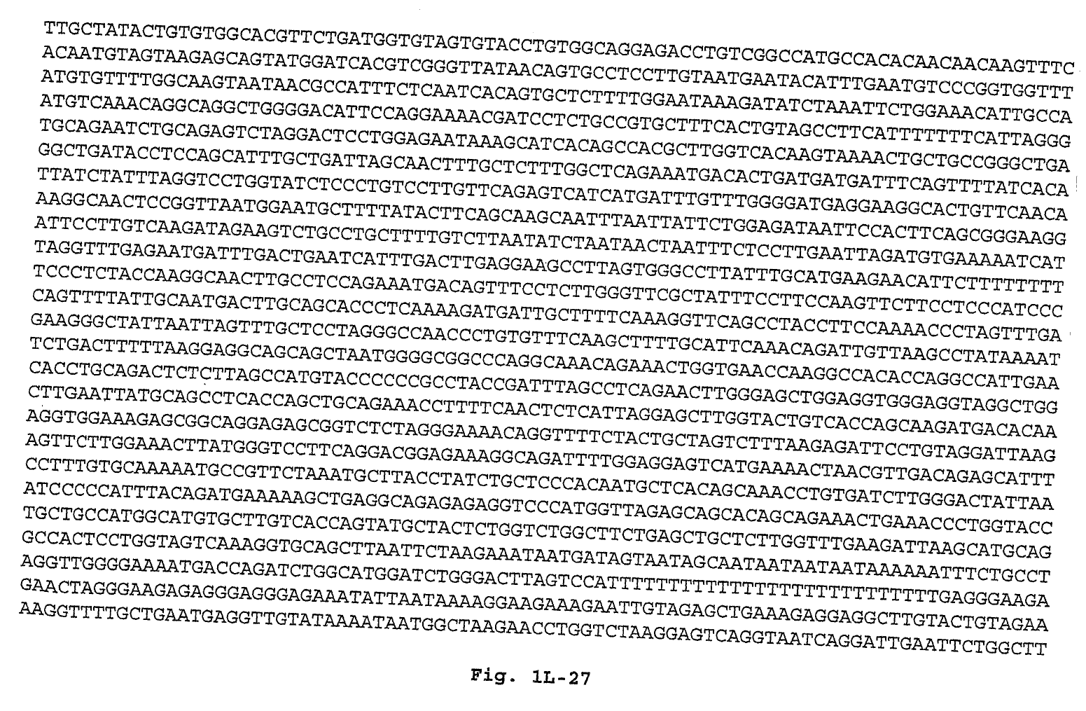

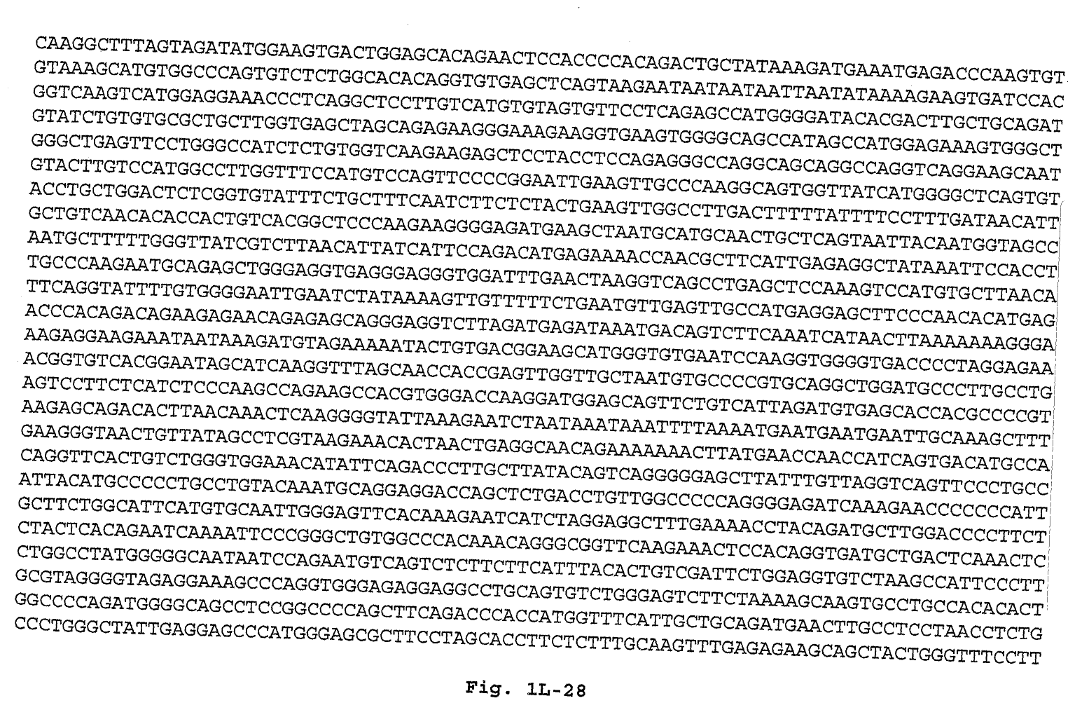

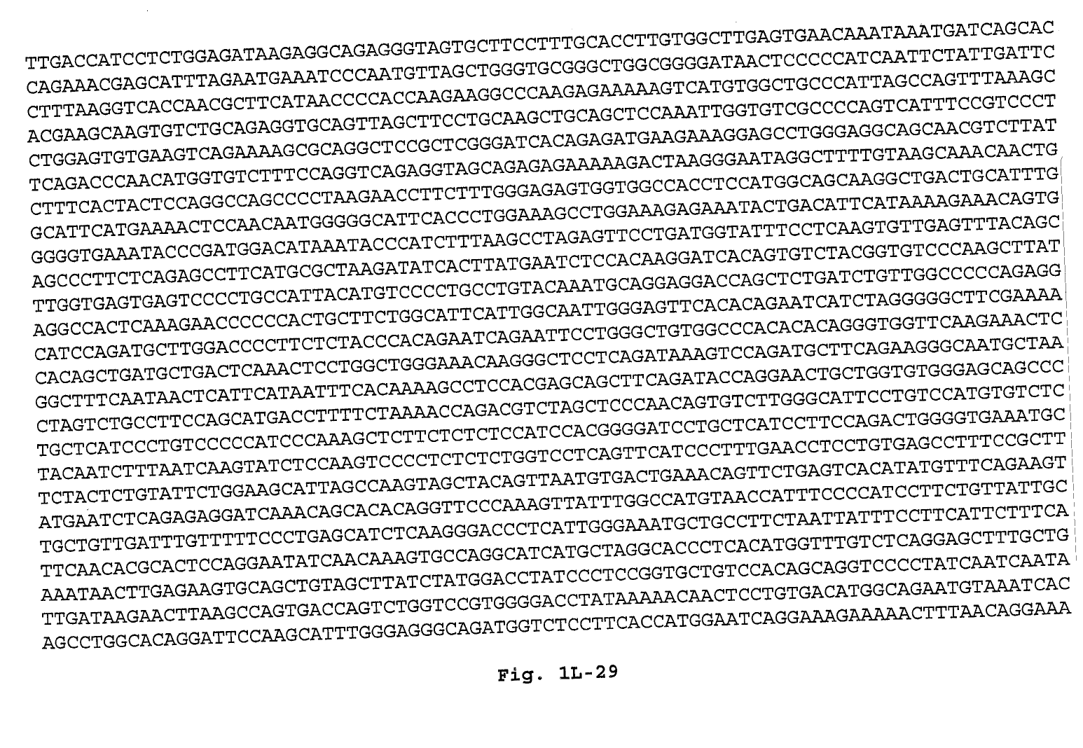

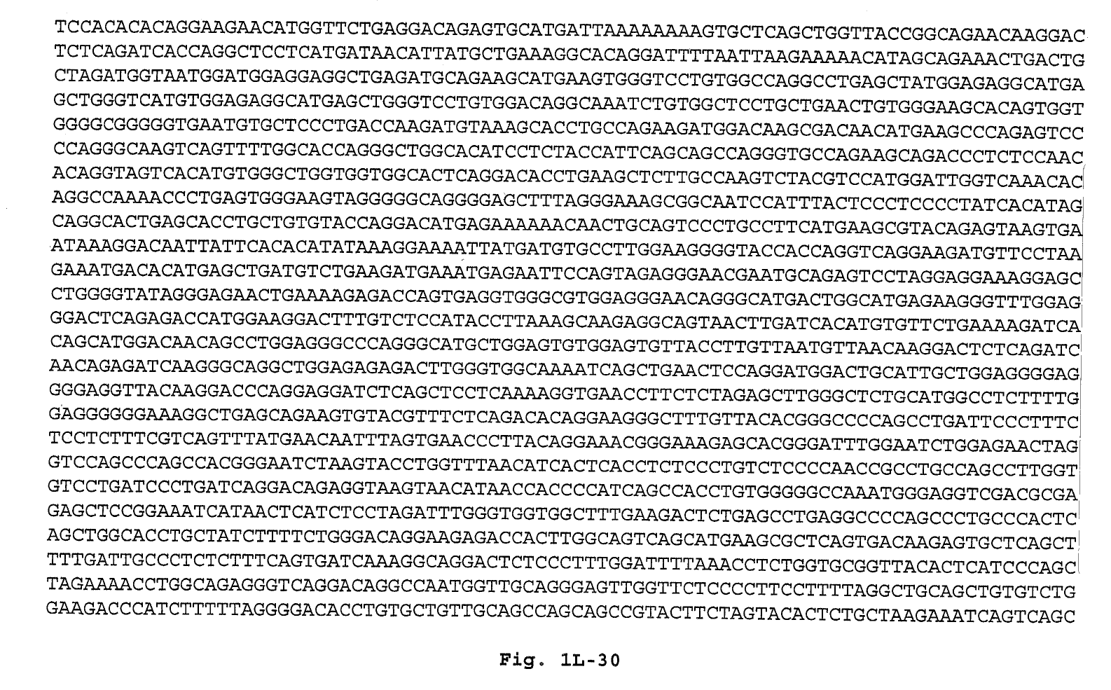

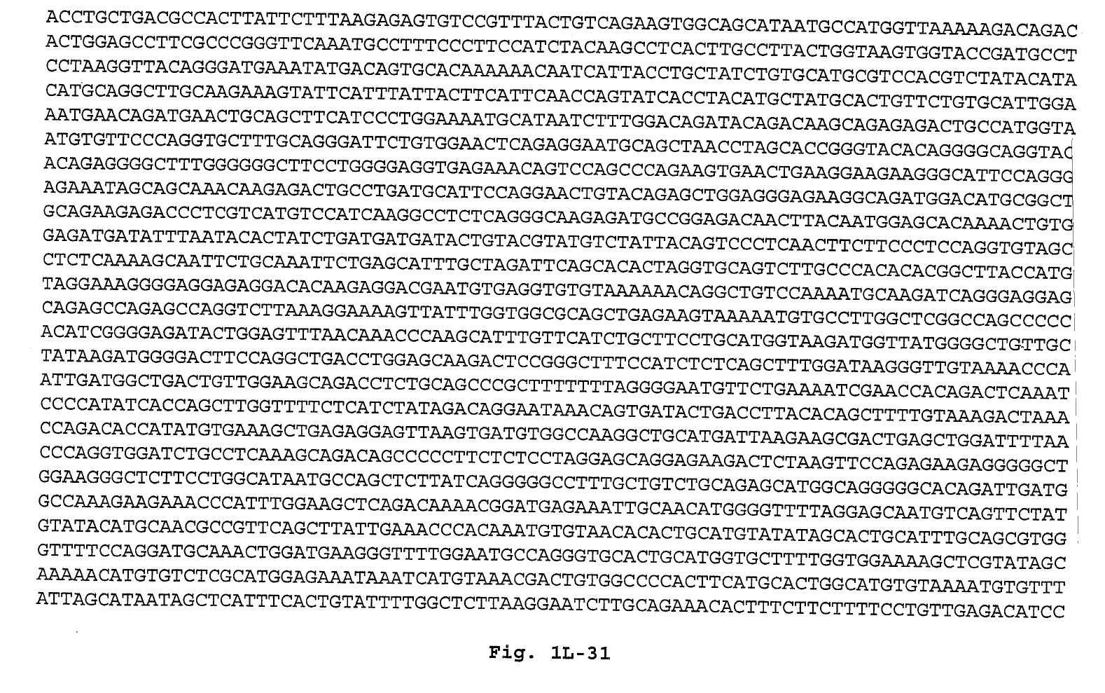

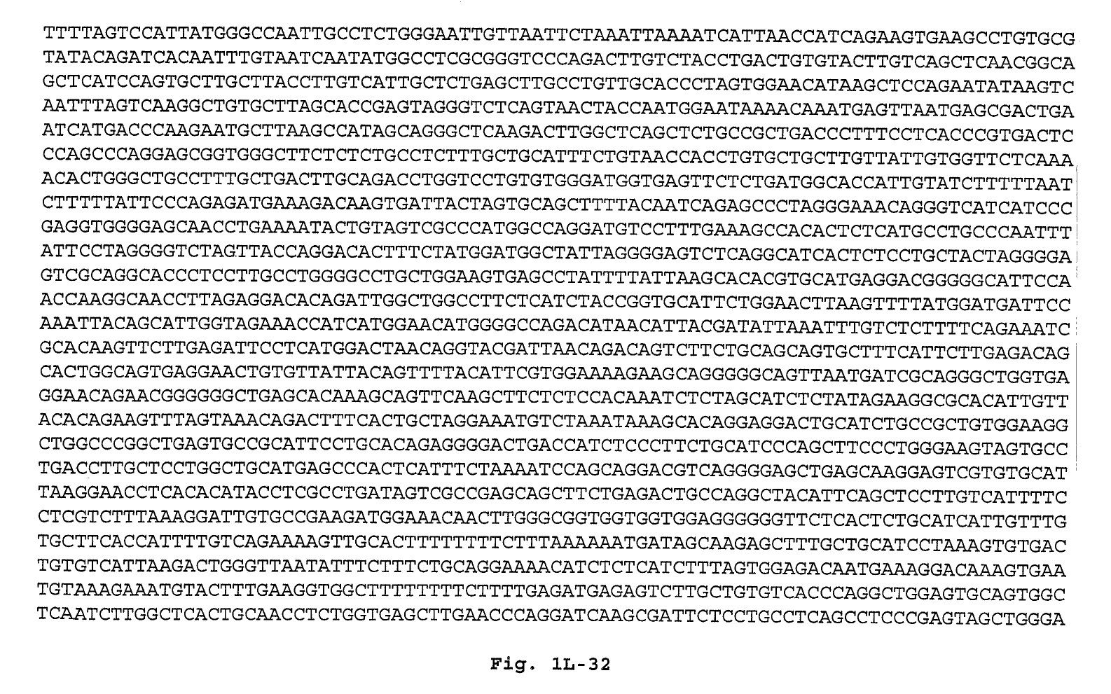

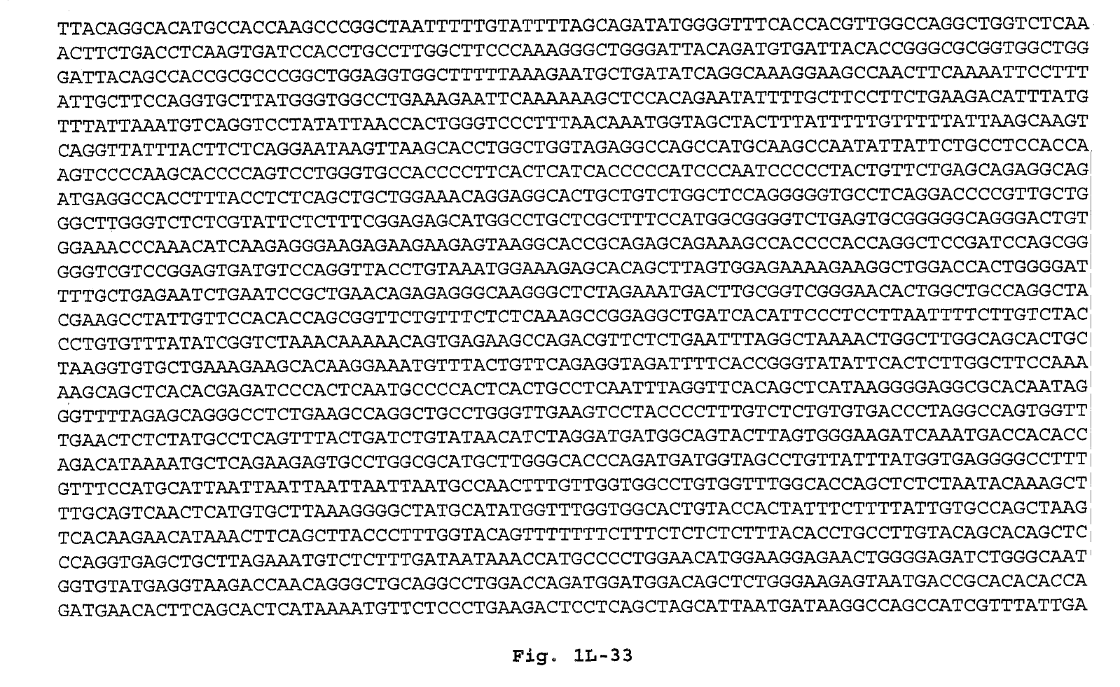

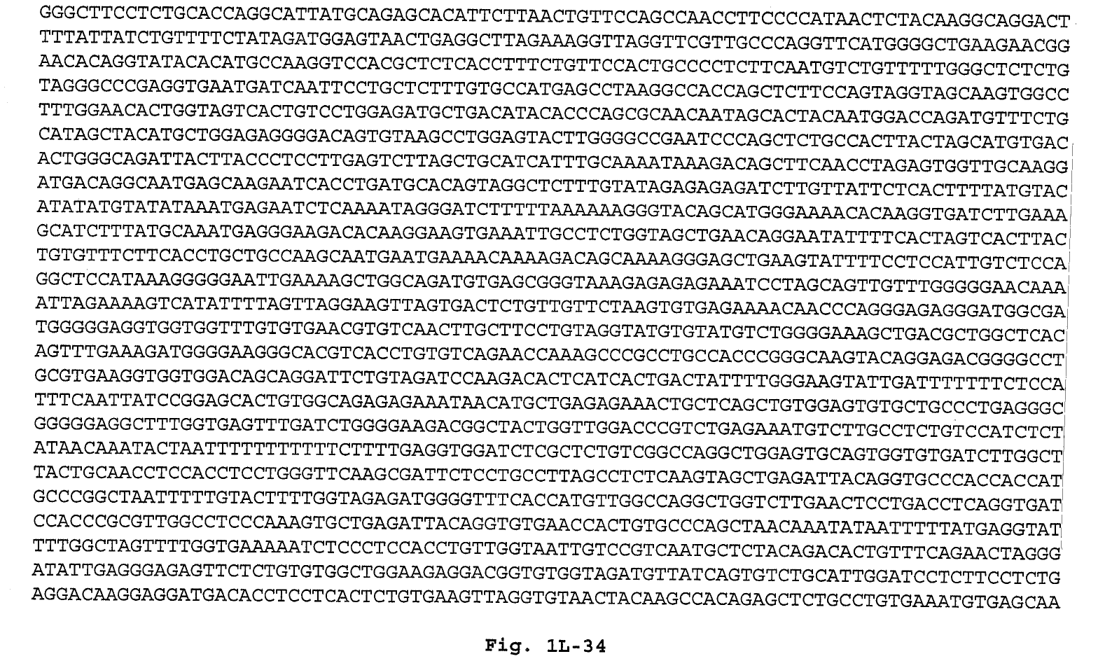

The full length of the cDNA encoding human protein 65h2 (FIG. 1; SEQ ID NO: 1) is 2563 nucleotide residues. The ORF of this cDNA, nucleotide residues 42 to 1970 of SEQ ID NO: 1 (i.e. SEQ ID NO: 2), encodes a 643-amino acid protein (FIG. 1; SEQ ID NO: 3) which exhibits amino acid sequence homology with HPT protein and other prostaglandin transporters. A human genomic sequence (FIG. 1L; nucleotide residues 1-50,000 in SEQ ID NO: 4, nucleotide residues 50,001-31,124 in SEQ ID NO: 12) corresponding to protein 65h2 is shown in FIG. 1L. The gene encoding human protein 65h2 maps to human chromosome 15 at q26.1. A PAC clone including this region has been sequenced, and the sequence of that clone is listed in GenBank Accession number AC005319. It was not previously recognized that any protein, let alone protein 65h2 was encoded within the portion of the genome encompassed by the PAC clone. The exon and intron structure of the genomic sequence is described in Tables I and II. Table I lists the positions of exons in this sequence, and Table II lists intron positions and branch sites (bold residues in Table II indicate RNA splicing junctions.

TABLE I

| | | | Corresponding |
|---|---|---|---|
| | | | Amino Acid |
| Exon | Position within | Position within | Sequence (Residues of |
| Designation | SEQ ID NO: 1 | SEQ ID NO: 4/12 | SEQ ID NO: 3) |
| a | 541-639 | 3683-3781 | 168-199 |
| b | 640-903 | 13078-13341 | 200-287 |
| c | 904-1068 | 29276-29440 | 288-342 |
| d | 1069-1267 | 34872-35070 | 343-408 |
| e | 1268-1406 | 37163-37301 | 409-455 |
| f | 1407-1582 | 55668-55843 | 456-513 |
| g | 1583-1647 | 59634-59698 | 514-535 |
| h | 1648-1890 | 71440-71682 | 536-616 |
| i | 1891-2546 | 80469-81124 | 617-643 |

TABLE II

| Intron | Position in SEQ | Donor Site | Acceptor Site | Branch Site(s) |
|---|---|---|---|---|
| Designation | ID NO:4/12 | Sequence | Sequence | (TACTAAC) |
| i | 0-3682 | | TCAG | |
| ii | 3782-13077 | GTAA | ACAG | 7141-7147 |
| iii | 13342-29275 | GTAA | GCAG | |
| iv | 29441-34871 | GTGA | CCAG | |
| v | 35071-37162 | GTGA | CCAG | |
| vi | 37302-55667 | GTAA | TCAG | 39794-39800, |
| | | | | 52196-52202 |
| vii | 55844-59633 | GTAA | GTAG | |
| viii | 59699-71439 | GTAT | ACAG | |
| ix | 71683-80468 | GTGA | TTAG | |

In addition to full length human protein 65h2, the invention includes fragments, derivatives, and variants of protein 65h2, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 1 or some portion thereof, such as the portion which encodes human protein 65h2, or a domain, fragment, derivative, or variant of protein 65h2. These nucleic acids are collectively referred to as nucleic acids of the invention.

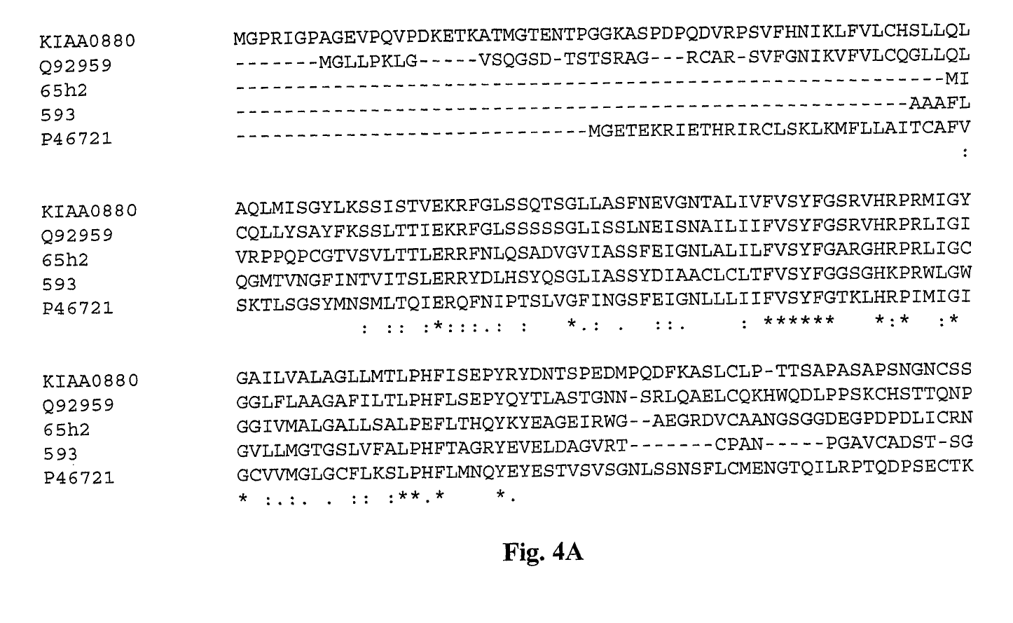

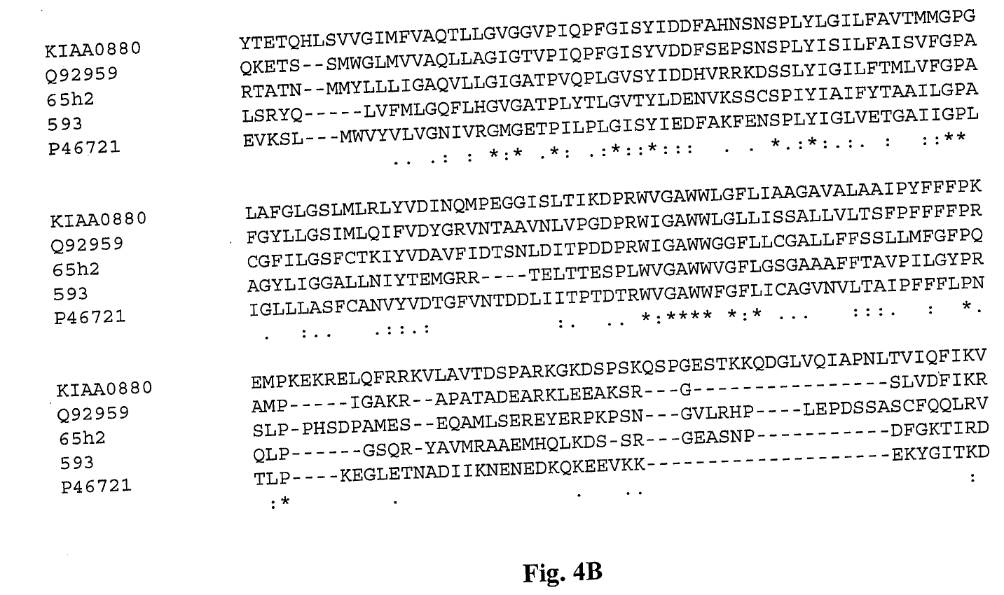

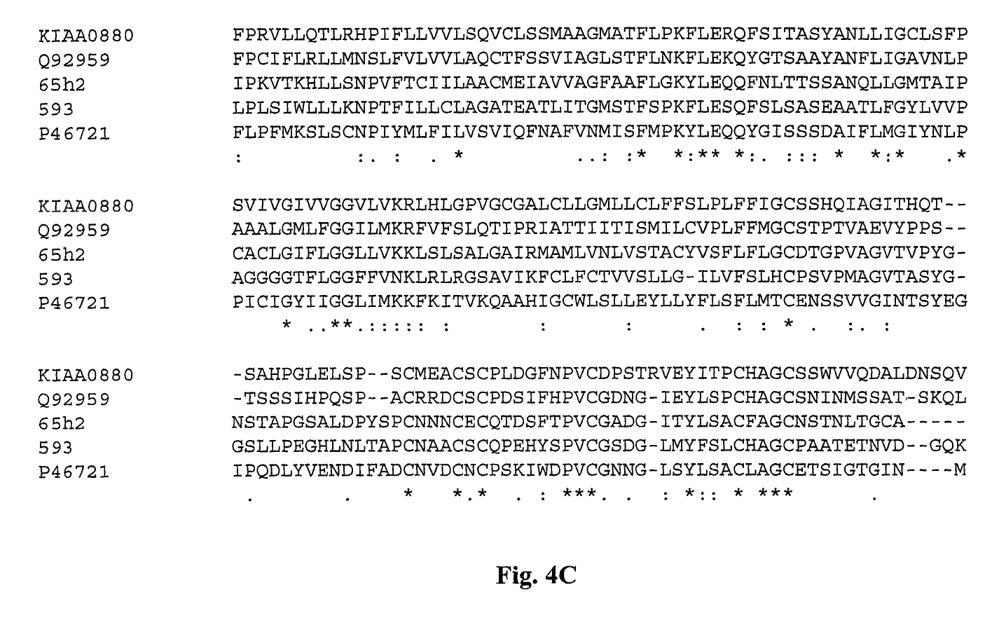

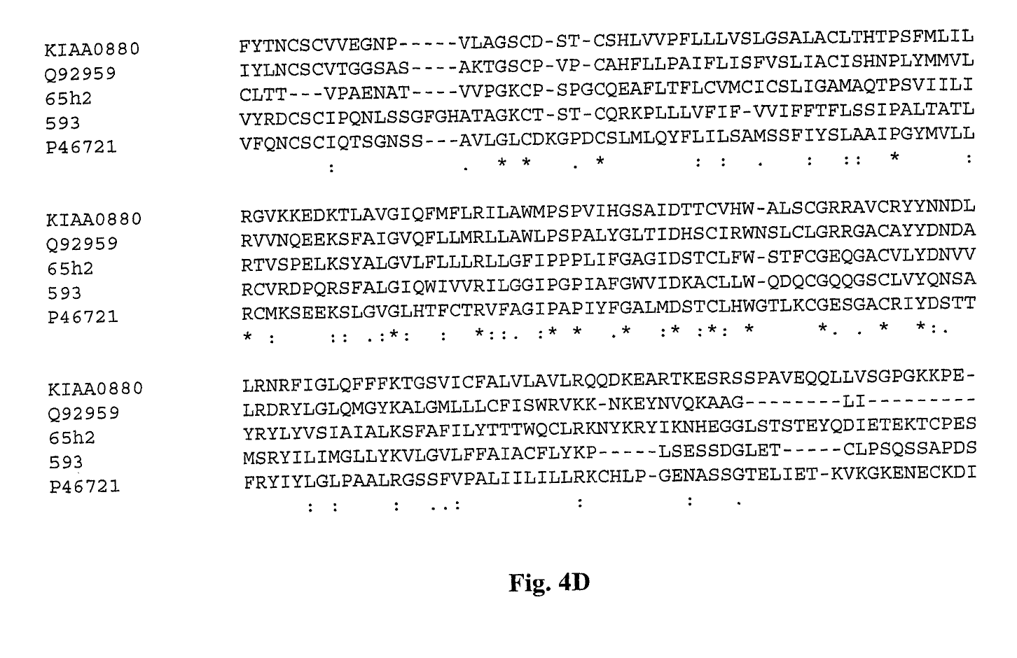

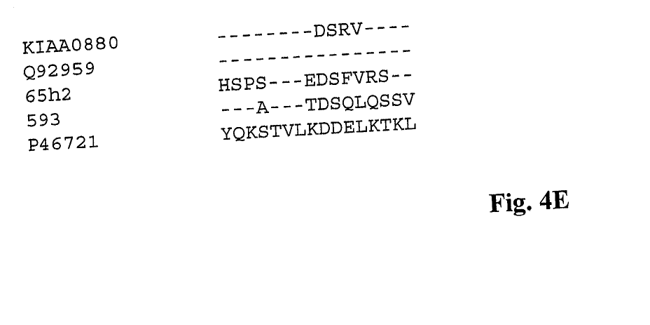

65h2 proteins of the invention and nucleic acid molecules encoding them comprise a family of molecules having certain conserved structural and functional features, as indicated by the conservation of amino acid sequence between protein 65h2 and HPT (SEQ ID NO: 10), as shown in FIGS. 1I through 1K and in FIGS. 4A through 4E, in which the amino acid sequence of human protein 65h2 is aligned with those of HPT, the human OatP sodium-independent organic anion transporter protein (GenBank Accession no. P46721; SEQ ID NO: 11), human KIAA0880 protein (GenBank Accession no. 4240248; SEQ ID NO: 9), and human protein 593 (as described herein, SEQ ID NO: 7).

65h2 proteins typically comprise a variety of potential post-translational modification sites (often within an extracellular domain), such as those described herein in Table III, as predicted by computerized sequence analysis of human 65h2 protein using amino acid sequence comparison software (comparing the amino acid sequence of protein 65h2 with the information in the PROSITE database {rel. 12.2; Feb, 1995} and the Hidden Markov Models database {Rel. PFAM 3.3}). In certain embodiments, a protein of the invention has at least 1, 2, 4, 6, 8, 10, 15, or 20 or more of the post-translational modification sites listed in Table III.

TABLE III

| Type of Potential Modification Site | Amino Acid Residues of | Amino Acid |
|---|---|---|
| or Domain | SEQ ID NO:3 | Sequence |
| N-glycosylation site | 104 to 107 | NGSG |
| | 120 to 123 | NRTA |
| | 332 to 335 | NLTT |
| | 408 to 41 | NSTA |
| | 453 to 456 | NSTN |
| | 470 to 473 | NATV |
| cAMP- or cGMP-dependent protein | 159 to 162 | RKDS |
| kinase phosphorylation site | 362 to 365 | KKLS |
| Protein kinase C phosphorylation site | 256 to 258 | SER |
| | 625 to 627 | TEK |
| Casein kinase II phosphorylation site | 16 to 19 | TTLE |
| | 34 to37 | SSFE |
| | 106 to 109 | SGGD |
| | 151 to 154 | SYID |
| | 200 to 203 | SNLD |
| | 205 to 208 | TPDD |
| | 256 to 259 | SERE |
| | 414 to 417 | SALD |
| | 616 to 619 | TSTE |
| | 628 to 631 | TCPE |
| | 634 to 637 | SPSE |
| Tyrosine kinase phosphorylation site | 158 to 165 | RRKDSSLY |
| N-myristoylation site | 30 to 35 | GVIASS |
| | 64 to 69 | GIVMAL |
| | 70 to 75 | GALLSA |
| | 167 to 172 | GILFTM |
| | 184 to 189 | GSFCTK |
| | 213 to 218 | GAWWGG |
| | 353 to 358 | GIFLGG |
| | 451 to 456 | GCNSTN |
| | 482 to 487 | GCQEAF |
| | 547 to 552 | GIDSTC |
| | 612 to 617 | GGLSTS |
| Sugar (or other) transport domain | 2 to 446 | See FIG. 1 |
| Kazal domain | 426 to 460 | See FIG. 1 |

Protein 65h2 comprises domains which exhibit homology with known sugar (or other) transport domains and with Kazal domains. In one embodiment, the protein of the invention has at least one domain that is at least 55%, preferably at least about 65%, more preferably at least about 75%, yet more preferably at least about 85%, and most preferably at least about 95% identical to one of these domains. Preferably, the protein of the invention has at least two domains, each of which is at least 55%, preferably at least about 65%, more preferably at least about 75%, yet more preferably at least about 85%, and most preferably at least about 95% identical to either the sugar (or other) transport domain or the Kazal domain of protein 65h2.

Sugar (or other) transport domains occur in a variety of proteins involved in transmembrane transport of sugars and other metabolites. Other proteins which comprise such a domain include human glucose transporters GLUT1, GLUT2, GLUT3, GLUT4, GLUT5, GLUT6, and GLUT7, Escherichia coli proteins AraE (arabinose-proton symporter), GalP (galactose-proton symporter), citrate-proton symport protein, KgtP (α-ketoglutarate permease), ProP (proline/betaine transporter), and XylE (xylose-proton symporter), Escherichia coli hypothetical proteins YabE, YdjE, and YhjE, Klebsiella pneumoniae citrate-proton symport protein, Zymomonas mobilis glucose facilitated diffusion protein, yeast high and low affinity glucose transport proteins (SNF3 and HXT1 through HXT14), yeast galactose transporter, yeast maltose permease, yeast myo-inositol transporter, yeast carboxylic acid transporter homolog JEN1, yeast hypothetical proteins YBR241c, YCR98c, and YFL040w, Klyveromyces lactis lactose permease, Neurospora crassa quinate transporter, Emericella nidulans quinate permease, Chlorella hexose carrier, Arabidopsis thaliana glucose transporter, spinach sucrose transporter, Leishmania donovani transporters D1 and D2, Leishmania enriettii probable transport protein LTP, Caenorhabditis elegans hypothetical protein ZK637.1, Haemophilus influenzae hypothetical proteins H10281 and HI0418, and Bacillus subtilis hypothetical proteins YxbC and YxdF. Occurrence of a sugar (or other) transport domain in protein 65h2 indicates that protein 65h2 is involved in transmembrane transport of one or more compounds, most likely a compound having a molecular weight on the order of a hexose or greater (i.e. having a molecular weight greater than about 180). Examples of such compounds include prostaglandins, thromboxanes, hexoses, disaccharides, hormones (e.g. insulin), peptides, neurotransmitters, cytokines, chemokines, and the like. Protein 65h2 thus mediates one or more of facilitated diffusion and symport or antiport (e.g. involving co-transport of a proton, a sodium ion, a potassium ion, or another physiological ion).

Kazal domains occur frequently in serine protease inhibitors. However, these domains also occur as extracellular domains in agrins, which are not thought to have roles as protease-inhibitors. These domains are characterized by occurrence, preferably within an extracellular domain, of the consensus pattern

—C—X_{(7 or 8)}—C—X₆—Y—X₃—C—X_{(2 or 3)}—C—

wherein standard single-letter amino acid residue codes are used, X being any amino acid residue, and subscripts referring to the number of residues. Agrins are involved in organization of neural synapses, including, for example, inter-neuronal synapses within the central nervous system (e.g. glutamatergic synapses) and neuromuscular junctions (Martin and Sanes (1997) Development 124:3909-3917; Lieth and Fallon (1993) J. Neurosci. 13:2509-2514). Agrins are also involved in organization of endothelial cells and astrocytes during formation and maintenance of the blood brain barrier. Thus, occurrence of a Kazal domain in protein 65h2 indicates that this protein is involved in formation and maintenance of cell-to-cell interactions, and more particularly that the protein is involved in forming and maintaining neural synapses, including both neuron-to-neuron synapses and neuron-to-non-neural cell synapses (e.g. neuromotor and neuroendocrine synapses).

Human protein 65h2 exhibits sequence similarity to HPT (GenBank Accession no. Q92959), as indicated herein in FIGS. 1I through 1K. FIGS. 1I through 1K depict an alignment of the amino acid sequences of human protein 65h2 (SEQ ID NO: 3) and HPT (SEQ ID NO: 10). In this alignment (made using the ALIGN program of the GCG software package, pam120.mat scoring matrix, gap penalties −12/−4), the amino acid sequences of the proteins are 32.4% identical.

Protein 65h2 is predicted by computerized amino acid sequence analysis (using the MEMSAT computer program) to be a twelve-transmembrane region integral membrane protein having transmembrane regions at approximately the following positions within SEQ ID NO: 3.

1) from about amino acid residue 8 to about residue 17;

2) from about amino acid residue 29 to about residue 52;

3) from about amino acid residue 59 to about residue 76;

4) from about amino acid residue 129 to about residue 153;

5) from about amino acid residue 164 to about residue 186;

6) from about amino acid residue 215 to about residue 236;

7) from about amino acid residue 301 to about residue 324;

8) from about amino acid residue 341 to about residue 361;

9) from about amino acid residue 374 to about residue 392;

10) from about amino acid residue 490 to about residue 513;

11) from about amino acid residue 524 to about residue 548; and

12) from about amino acid residue 575 to about residue 592.

Extracellular domains are predicted to include approximately amino acid residues 18 to 28, 77 to 128, 187 to 214, 325 to 340, 393 to 489, and 549 to 574 of SEQ ID NO: 3. Intracellular domains are predicted to include approximately amino acid residues 1 to 7, 53 to 58, 154 to 163, 237 to 300, 362 to 373, 514 to 523, and 593 to 643 of SEQ ID NO: 3.

Human protein 65h2 can have additional amino acid residues at the amino terminal end of the sequence listed in SEQ ID NO: 3 (i.e. the protein can have an additional portion at its amino terminus). For example, protein 65h2 can have 1, 2, 4, 6, 10, 15, 20, 25, or 30 or more additional amino acid residues at the amino terminus indicated in SEQ ID NO: 3.

FIG. 1M depicts a variety of plots produced by computerized analysis of the amino acid sequence of human protein 65h2. Regions of the protein which are predicted to assume alpha helix (FIG. 1M a)), beta sheet (FIG. 1M c)), turn (FIG. 1M e)), and random coil (FIG. 1M g)) configurations by the Garnier-Robson method are indicated, as are regions predicted to assume alpha helix (FIG. 1M b)), beta sheet (FIG. 1M d)), and turn (FIG. 1M f)) configurations by the Chou-Fasman method. FIG. 1M h) is a Kyte-Doolittle hydrophilicity plot of human protein 65h2, wherein relatively hydrophilic regions are above the horizontal axis (value=0) and relatively hydrophobic regions are below the horizontal axis. FIG. 1M indicates amphipathic regions of the protein which are predicted by the methods of Eisenberg and Karplus-Schulz to assume alpha (FIG. 1M i)), beta (FIG. 1M j)), and flexible (FIG. 1M k)) configurations. FIGS. 1M l) and m) are plots of the antigenic index, as calculated by the method of Jameson-Wolf and the surface probability, as calculated by the method of Emini, respectively.

FIG. 1N depicts a hydrophilicity plot of human protein 65h2. Relatively hydrophobic regions are above the dashed horizontal line, and relatively hydrophilic regions are below the dashed horizontal line. As described elsewhere herein, relatively hydrophilic regions are generally located at or near the surface of a protein, and are more frequently effective immunogenic epitopes than are relatively hydrophobic regions. For example, the region of human protein 65h2 from about amino acid residue 415 to about amino acid residue 430 appears to be located at or near the surface of the protein, while the region from about amino acid residue 440 to about amino acid residue 450 appears not to be located at or near the surface.

The predicted molecular weight of human protein 65h2 is about 69.2 kilodaltons.

A monkey cDNA clone having significant homology with the human cDNA clone encoding protein 65h2 was isolated from a monkey brain cDNA library, indicating that human protein 65h2 is expressed in brain tissue, although it can, of course, be expressed in other tissues as well.

Biological function of human 65h2 proteins, nucleic acids encoding them, and modulators of these molecules

Human 65h2 proteins are involved in disorders which affect both tissues in which they are normally expressed and tissues in which they are normally not expressed. Based on the observation that 65h2 protein is expressed in monkey brain and is therefore likely expressed in human brain tissue, human 65h2 protein is involved in one or more biological processes which occur in brain and other neurological tissues. In particular, 65h2 is involved in modulating growth, proliferation, survival, differentiation, and activity of cells including, but not limited to, central nervous system neurons, peripheral nervous system neurons, motor neurons, sensory neurons, and sympathetic and parasympathetic neural cells of the animal in which it is normally expressed. Protein 65h2 is also involved in mediating interactions between cells, particularly between two neurons or between a neuron and a non-neuronal cell such as a muscle or endocrine cell. Thus, 65h2 protein has a role in disorders which affect neuronal cells and cells which interact with neurons and their growth, proliferation, survival, differentiation, and activity.

Widespread expression of 65h2 has been detected among human tissue types Thus, the growth- , proliferation- , survival- , differentiation- , and activity-modulating activities of 65h2 protein affect cells of many types. Thus, protein 65h2 can affect cell-to-cell interactions in a wide variety of cell types.

The presence of the sugar (or other) transport domain in protein 65h2 indicates that this protein is involved in transmembrane transport of one or more charged organic compounds such as prostaglandins, thromboxanes, neurotransmitters, hormones, small peptides, short polysaccharides (e.g. disaccharides), and the like. The proteins of the invention are therefore involved in one or more disorders relating to inappropriate uptake or release of such molecules (i.e. including inappropriate failure to take up or release such molecules). Protein 65h2 is thus involved in one or more of a variety of cellular uptake and release disorders such as diabetes, nutritional disorders (e.g. vitamin deficiencies, and malnutrition), metabolic disorders (e.g. obesity, porphyrias, hyper- and hypolipoproteinemia, lipidoses, and water, electrolyte, mineral, and acid/base imbalances), and neural transmission disorders (e.g. inappropriate pain, dementia, multiple sclerosis, nerve root disorders, Alzheimer's disease, Parkinson's disease, depression, physical and psychological substance addiction, sexual dysfunction, schizophrenic disorders, delusional disorders, mood disorders, sleep disorders, and the like).

Occurrence of a Kazal domain in human protein 65h2 further implicates this protein in neuronal development and transmission. The presence of this domain therefore indicates that 65h2 protein is involved in disorders relating to inappropriate formation (i.e. including failure to form) and maintenance (i.e. including deterioration) of neuronal synapses, including both neuron-to-neuron synapses and neuron-to-non-neuronal cell synapses. Thus, in addition to the neural transmission disorders described above, protein 65h2 is also implicated in disorders such as stroke, regeneration of chronically or traumatically damaged neuronal structures (including nerve, brain, and spinal cord), developmental neuronal disorders (e.g. spina bifida), neuronal cancers (e.g. gliomas, astrocytomas, ependymomas, pituitary adenomas, and the like), peripheral nerve deficit, cardiac insufficiency, and the like.

The observation that human protein 65h2 shares sequence homology with proteins involved in transmembrane prostaglandin transport indicates that 65h2 protein has activity identical or analogous to the activity of those proteins, i.e. that 65h2 catalyzes or facilitates transmembrane transport of one or more prostaglandins, thromboxanes, other hormones or hormone-like molecules, or other charged organic compounds. Exemplary molecules which can be transported across cell membranes via protein 65h2 include one or more charged organic compounds such as prostaglandins A₁, A₂, B₁, B₂, D₂, E₁, E₂, F_{1α}, F_{2α}, G₂, H₂, I₂, and J₂ and thromboxanes A₂ and B₂. Uptake and release of prostaglandins and thromboxanes, for example, are known to be involved in a variety of physiological processes and disorders including glaucoma, ovum fertilization, sperm motility, pregnancy, labor, delivery, abortion, gastric protection, peptic ulcer formation, intestinal fluid secretion, liver protection, liver damage, liver fibrosis, pain stimulation, glomerular filtration, maintenance of body temperature, fever, airway resistance, asthma, chronic obstructive pulmonary disorder, modulation of blood pressure, hypertension, shock, modulation of inflammation, platelet aggregation, abnormal blood coagulation, atherosclerosis, arteriosclerosis, and coronary artery disease. Thus, polypeptides and nucleic acid molecules of the invention, and compounds which bind with or modulate one or more polypeptides and nucleic acid molecules of the invention can be used to prognosticate, diagnose, inhibit, or treat one or more of the disorders listed above or one or more disorders associated with the physiological processes listed above.

### Protein 593

A cDNA encoding at least a portion of human 593 protein was identified by assembling isolated sequences derived from a library of human cDNA clones on the basis of homology with the nucleic acid sequence encoding human protein 65h2. Human protein 593 is predicted by structural analysis to be a transmembrane transporter protein having twelve transmembrane domains.

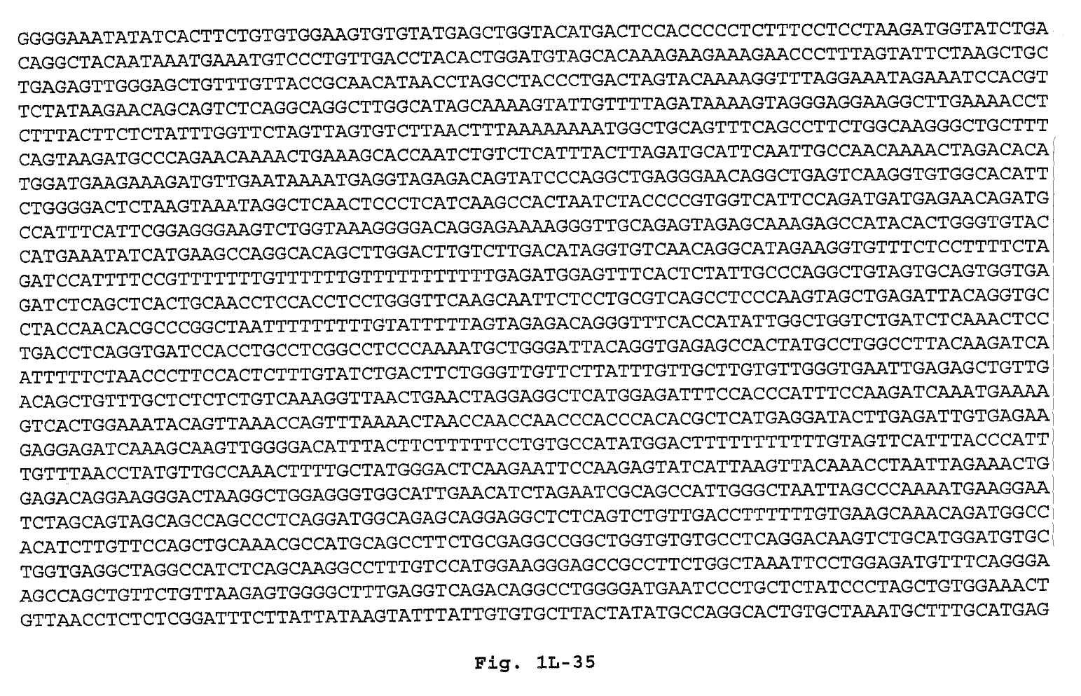

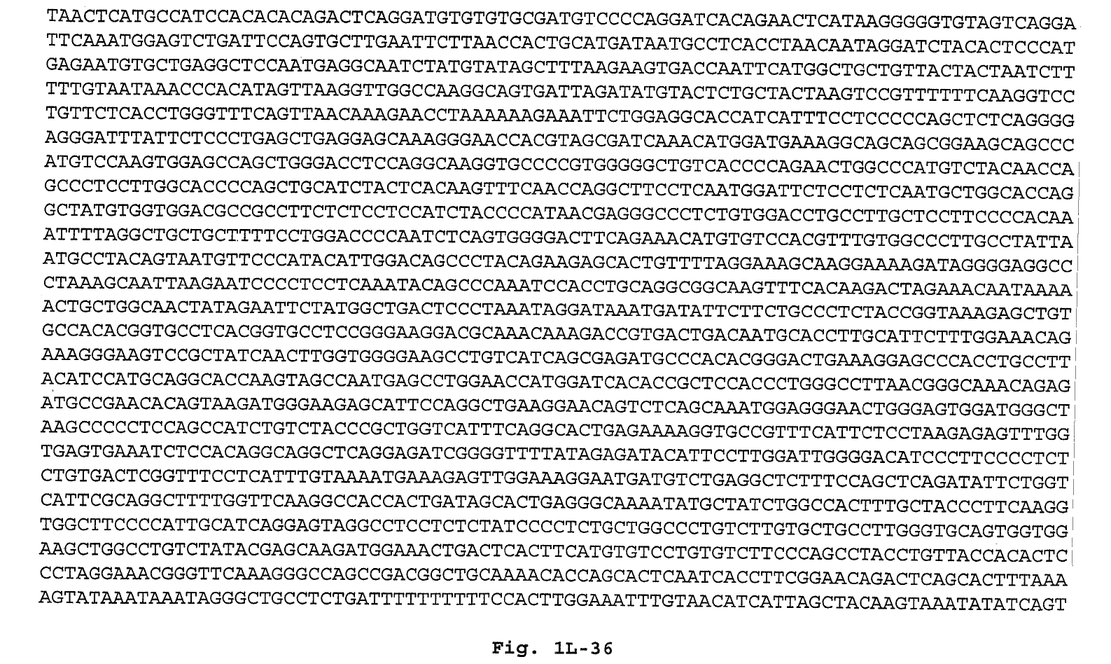

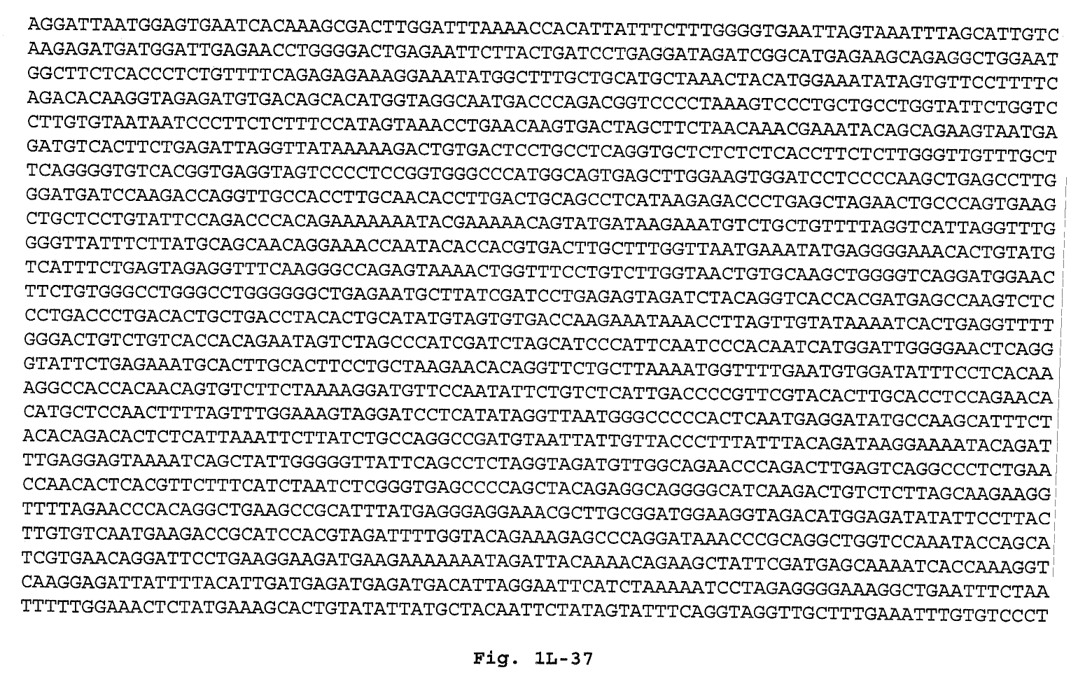

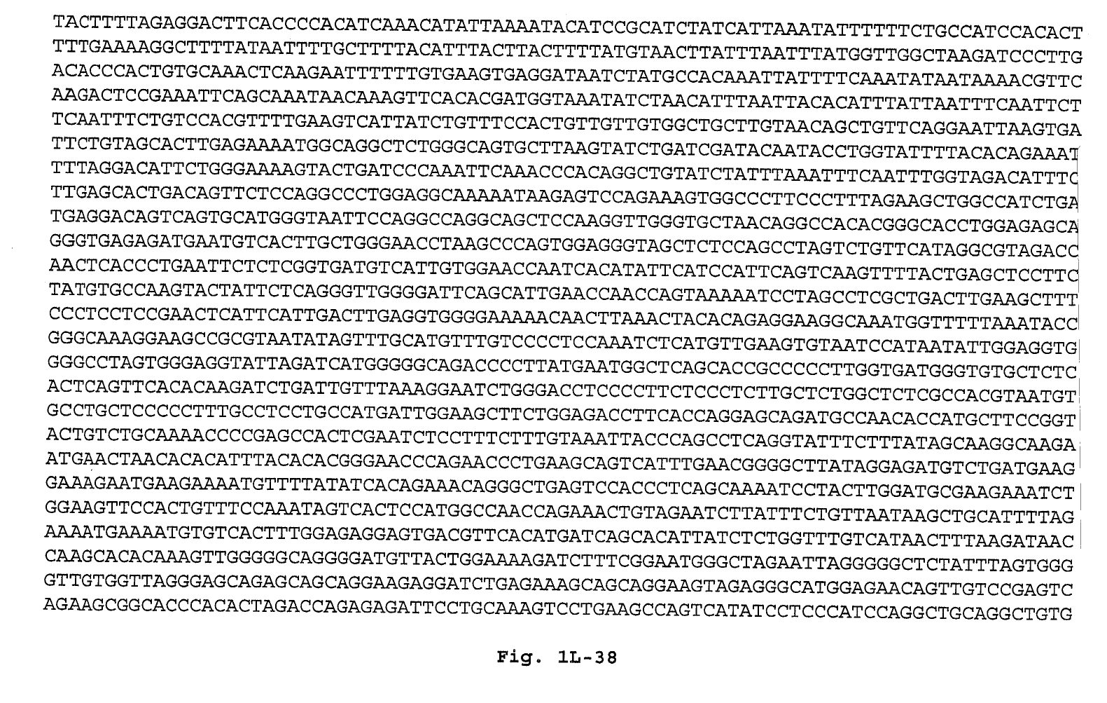

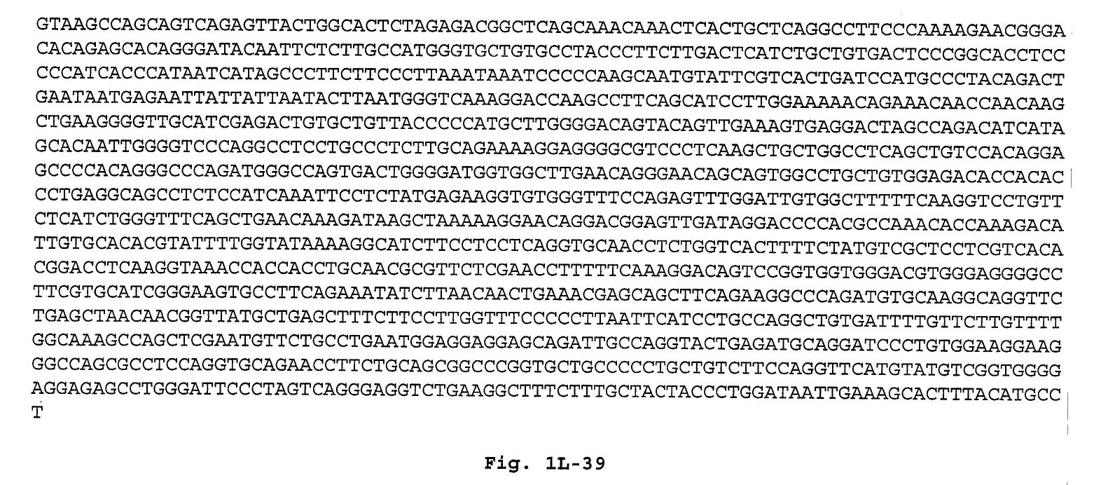

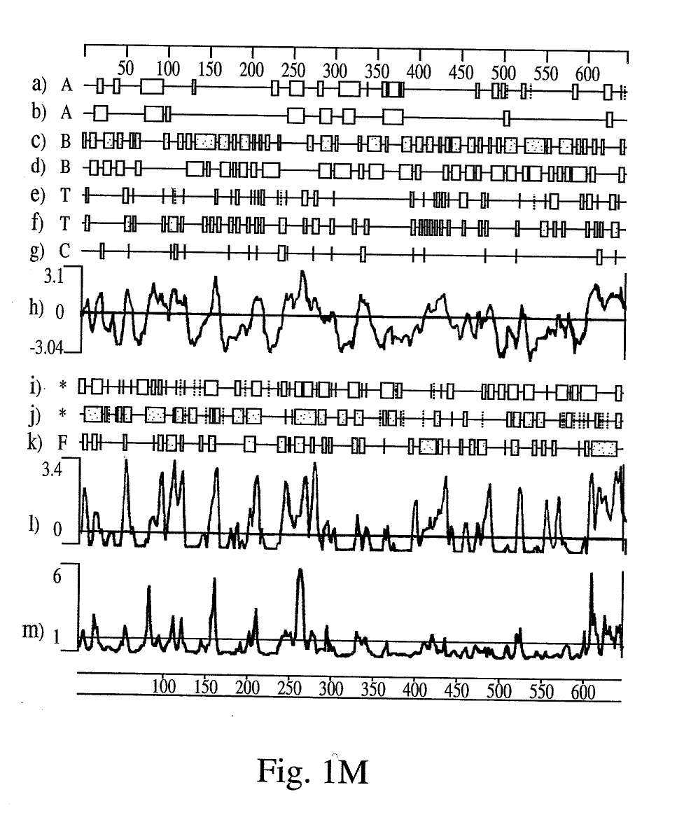

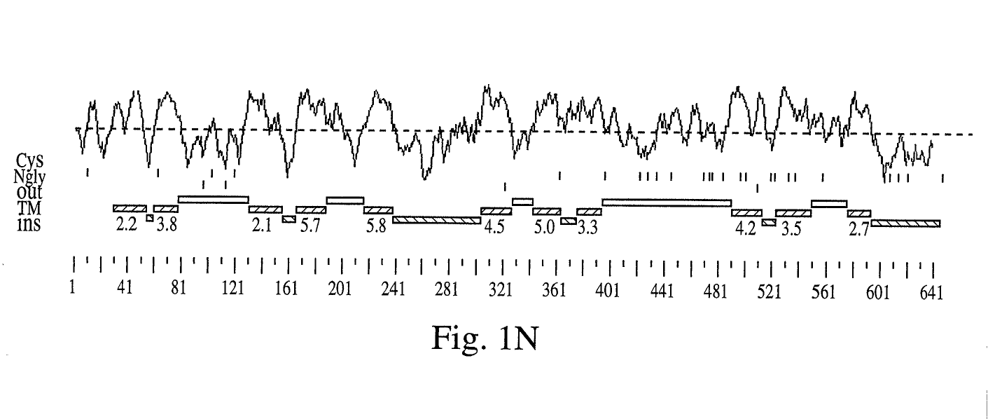

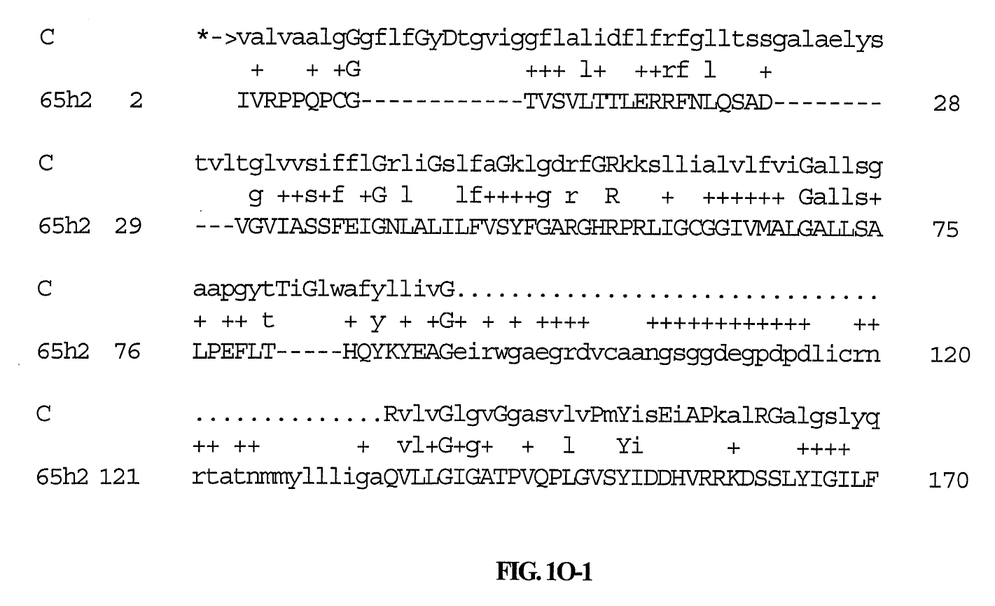

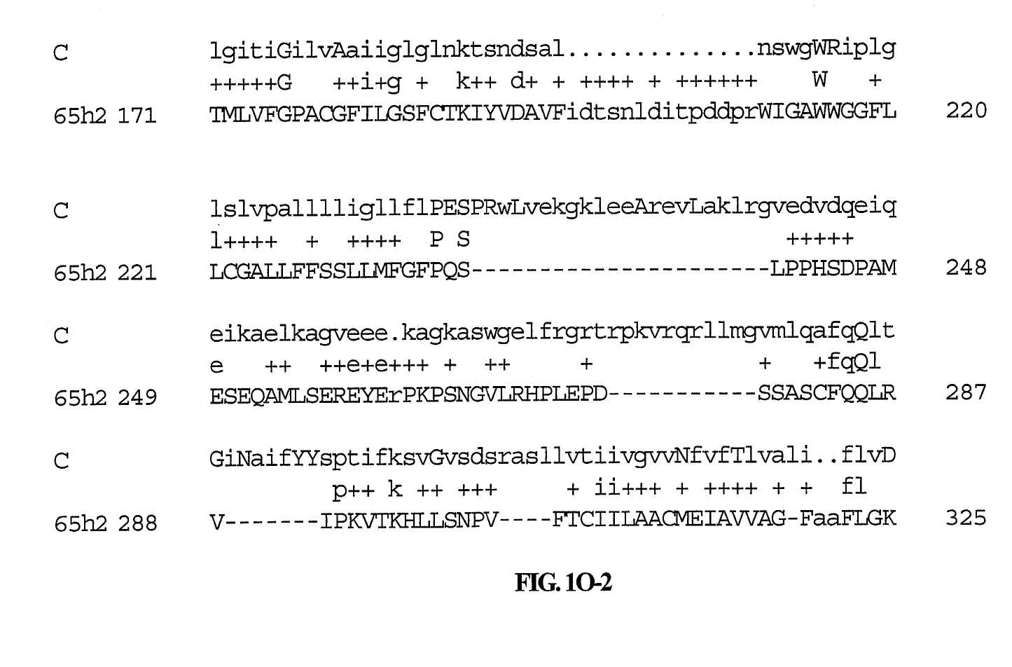

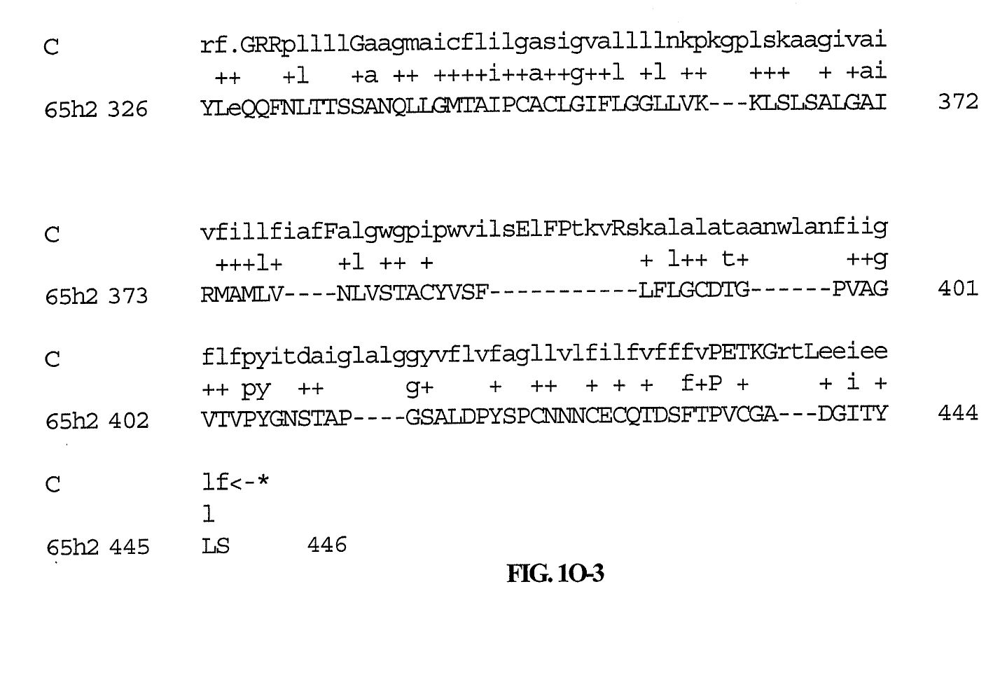

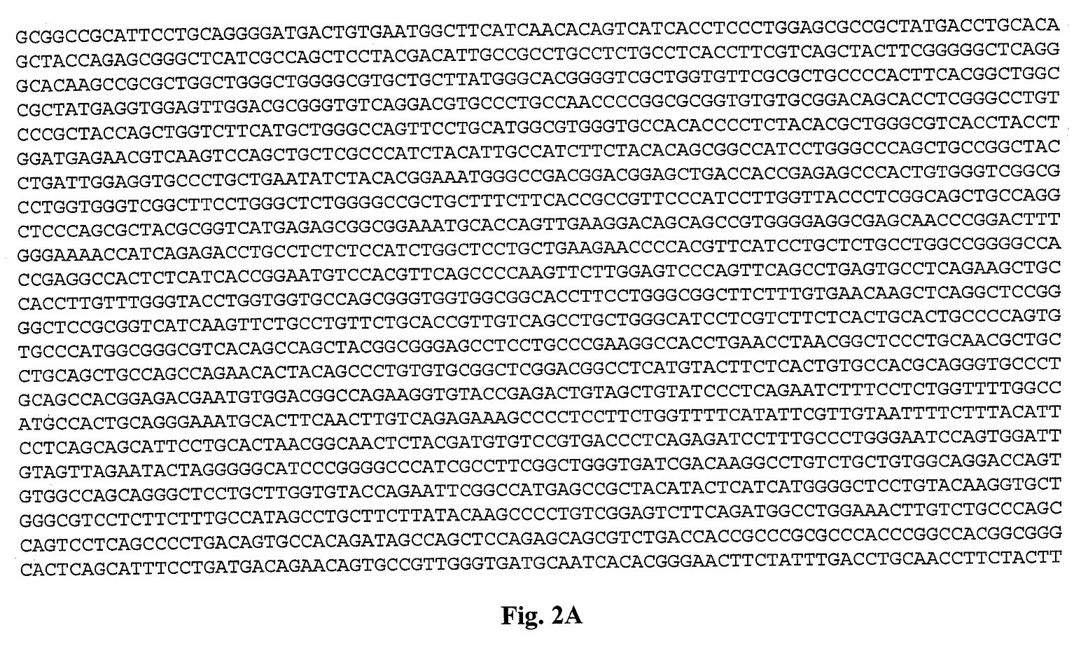

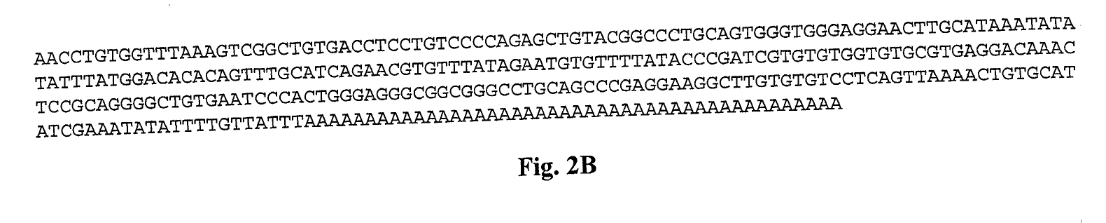

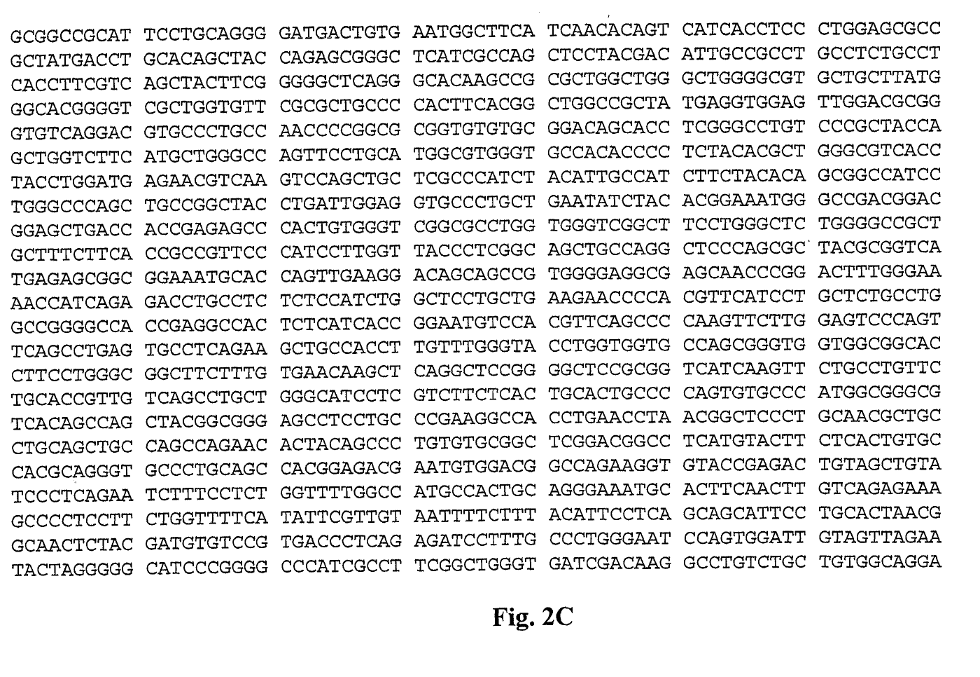

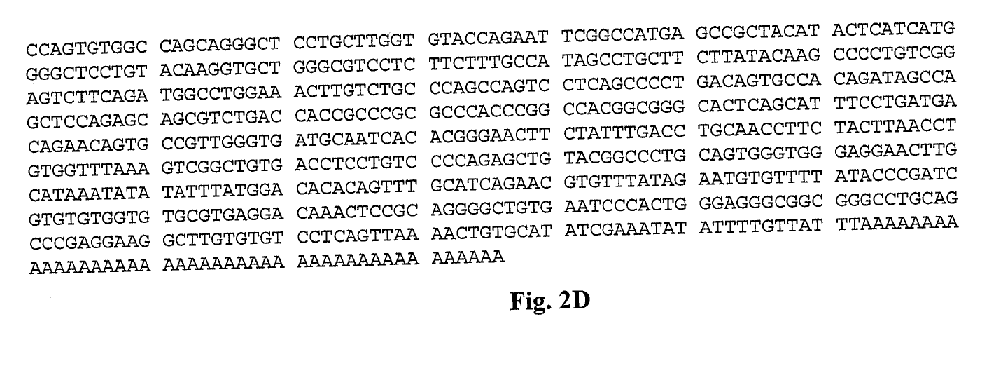

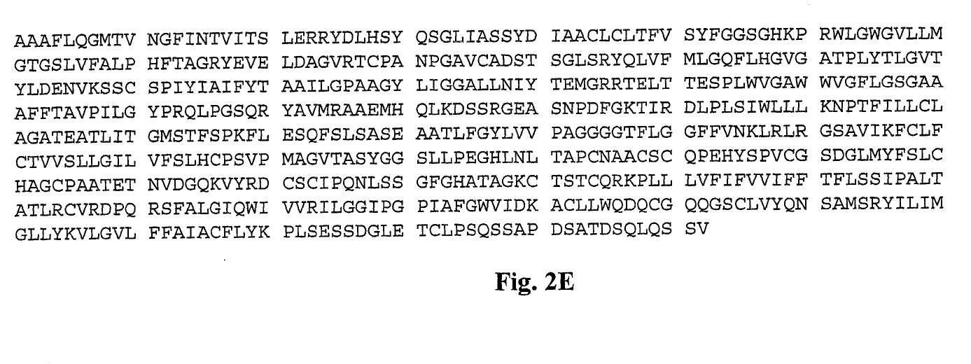

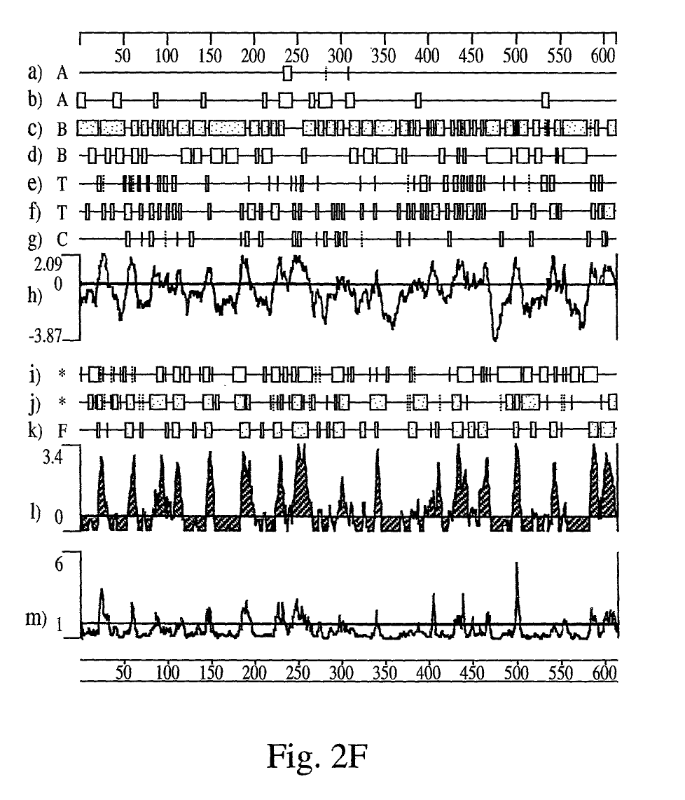

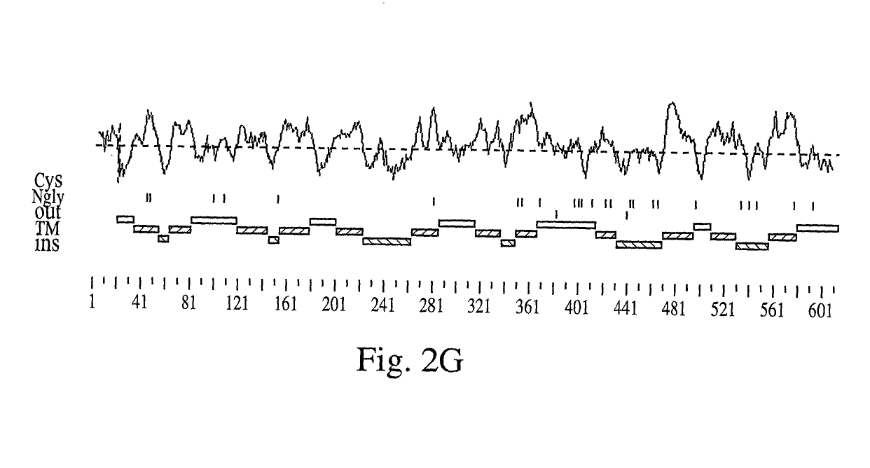

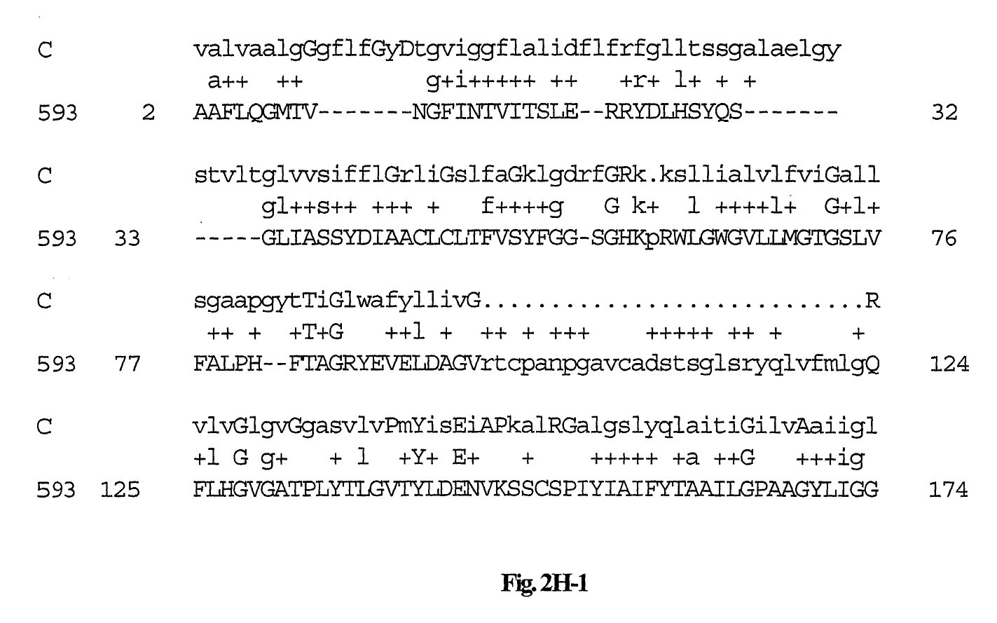

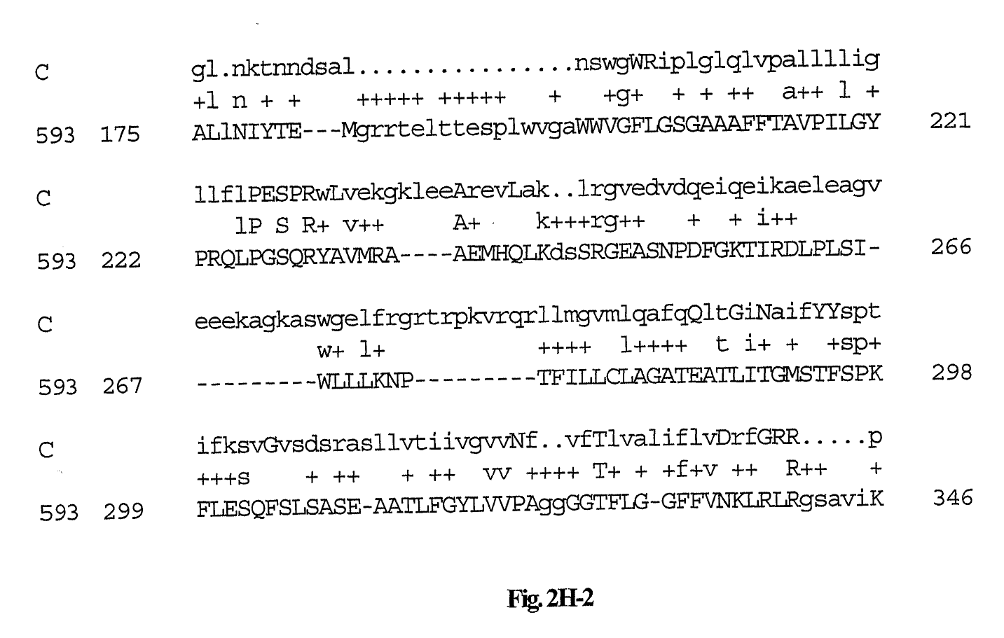

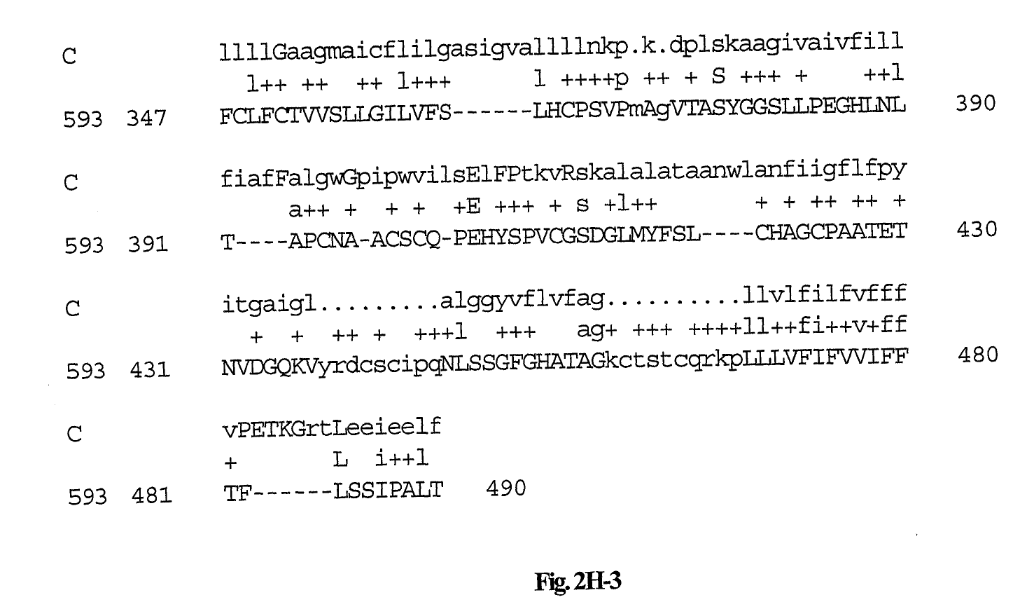

The full length of the cDNA encoding human protein 593 (FIG. 2; SEQ ID NO: 5) is 2276 nucleotide residues. The ORF of this cDNA, nucleotide residues 1 to 1836 of SEQ ID NO: 5 (SEQ ID NO: 6), encodes a 612-amino acid protein (FIG. 2; SEQ ID NO: 7) which exhibits amino acid sequence homology with human protein 65h2 and other prostaglandin transporters.

In addition to full length human protein 593, the invention includes fragments, derivatives, and variants of protein 593, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 5 or some portion thereof, such as the portion which encodes human protein 593, or a domain, fragment, derivative, or variant of protein 593. These nucleic acids are collectively referred to as nucleic acids of the invention.

Human 593 proteins of the invention and nucleic acid molecules encoding them comprise a family of molecules having certain conserved structural and functional features, as indicated in FIGS. 4A through 4E, in which the amino acid sequence of human protein 593 (SEQ ID NO: 7) is aligned with those of HPT (SEQ ID NO: 11), the human OatP sodium-independent organic anion transporter protein (GenBank Accession no. P46721; SEQ ID NO: 10), human KIAA0880 protein (GenBank Accession no. 4240248; SEQ ID NO: 9), and human protein 65h2 (as described herein, SEQ ID NO: 3).

Human 593 proteins typically comprise a variety of potential post-translational modification sites (often within an extracellular domain), such as those described herein in Table IV, as predicted by computerized sequence analysis of human 593 protein using amino acid sequence comparison software (comparing the amino acid sequence of protein 593 with the information in the PROSITE database {rel. 12.2; Feb; 1995} and the Hidden Markov Models database {Rel. PFAM 3.3}). In certain embodiments, a protein of the invention has at least 1, 2, 4, 6, 8, 10, 15, or 20 or more of the post-translational modification sites listed in Table IV.

TABLE IV

| Type of Potential Modification Site | Amino Acid Residues of | Amino Acid |
|---|---|---|
| or Domain | SEQ ID NO:7 | Sequence |
| N-glycosylation site | 389 to 392 | NLTA |
| | 447 to 450 | NLSS |
| Protein kinase C phosphorylation site | 228 to 230 | SQR |
| | 245 to 247 | SSR |
| | 258 to 260 | TIR |
| | 296 to 298 | SPK |
| | 492 to 494 | TLR |
| Casein kinase II phosphorylation site | 19 to 22 | TSLE |
| | 37 to 40 | SSYD |
| | 140 to 143 | TYLD |
| | 246 to 249 | SRGE |
| | 251 to 254 | SNPD |
| | 258 to 261 | TIRD |
| | 307 to 310 | SASE |
| | 430 to 433 | TNVD |
| | 598 to 601 | SAPD |
| | 602 to 605 | SATD |
| Tyrosine kinase phosphorylation site | 23 to 30 | RRYDLHSY |
| N-myristoylation site | 7 to 12 | GMTVNG |
| | 33 to 38 | GLIASS |
| | 103 to 108 | GAVCAD |
| | 174 to 179 | GALLNI |
| | 206 to 211 | GSGAAA |
| | 282 to 287 | GATEAT |
| | 323 to 328 | GGGGTF |
| | 373 to 378 | GVTASY |
| | 423 to 428 | GCPAAT |
| | 540 to 545 | GQQGSC |
| | 588 to 593 | GLETCL |
| Amidation site | 183 to 186 | MGRR |
| Aminotransferase class-V pyridoxal | 52 to 68 | YFGGSGHKP- |
| phosphate attachment site | | RWLGWGVL |
| Sugar (or other) transport domain | 2 to 490 | See FIG. 2 |
| Kazal domain | 398 to 4441 | See FIG. 2 |

Protein 593 comprises domains which exhibit homology with known sugar (or other) transport domains and with Kazal domains. In one embodiment, the protein of the invention has at least one domain that is at least 55%, preferably at least about 65%, more preferably at least about 75%, yet more preferably at least about 85%, and most preferably at least about 95% identical to one of these domains. Preferably, the protein of the invention has at least two domains, each of which is at least 55%, preferably at least about 65%, more preferably at least about 75%, yet more preferably at least about 85%, and most preferably at least about 95% identical to either the sugar (or other) transport domain or the Kazal domain of protein 593.

Sugar (or other) transport domains occur in a variety of proteins involved in transmembrane transport of sugars and other metabolites. Other proteins which comprise such a domain include human glucose transporters GLUT1, GLUT2, GLUT3, GLUT4, GLUT5, GLUT6, and GLUT7, Escherichia coli proteins AraE (arabinose-proton symporter), GalP (galactose-proton symporter), citrate-proton symport protein, KgtP (α-ketoglutarate permease), ProP (proline/betaine transporter), and XylE (xylose-proton symporter), Escherichia coli hypothetical proteins YabE, YdjE, and YhjE, Klebsiella pneumoniae citrate-proton symport protein, Zymomonas mobilis glucose facilitated diffusion protein, yeast high and low affinity glucose transport proteins (SNF3 and HXT1 through HXT14), yeast galactose transporter, yeast maltose permease, yeast myo-inositol transporter, yeast carboxylic acid transporter homolog JEN1, yeast hypothetical proteins YBR241c, YCR98c, and YFL040w, Klyveromyces lactis lactose permease, Neurospora crassa quinate transporter, Emericella nidulans quinate permease, Chlorella hexose carrier, Arabidopsis thaliana glucose transporter, spinach sucrose transporter, Leishmania donovani transporters D1 and D2, Leishmania enriettii probable transport protein LTP, Caenorhabditis elegans hypothetical protein ZK637.1, Haemophilus influenzae hypothetical proteins HI0281 and HI0418, and Bacillus subtilis hypothetical proteins YxbC and YxdF. Occurrence of a sugar (or other) transport domain in protein 593 indicates that protein 593 is involved in transmembrane transport of one or more compounds, most likely a compound having a molecular weight on the order of a hexose or greater (i.e. having a molecular weight greater than about 180). Examples of such compounds include prostaglandins, thromboxanes, hexoses, disaccharides, hormones (e.g. insulin), peptides, neurotransmitters, cytokines, chemokines, and the like. Protein 593 thus mediates one or more of facilitated diffusion and symport or antiport (e.g. involving co-transport of a proton, a sodium ion, a potassium ion, or another physiological ion). One, both, or neither of a glycosaminoglycan attached at the predicted glycosaminoglycan attachment site and a pyridoxal phosphate moiety attached at the predicted pyridoxal phosphate attachment site can, in conjunction with the amino acid sequence of protein 593, determine the specificity of the protein for transporting molecules across the membrane of a cell in which it is expressed.

Like human protein 65h2, as described above, human protein 593 comprises a Kazal domain. Occurrence of a Kazal domain in protein 593 indicates that this protein is involved in formation and maintenance of cell-to-cell interactions, and more particularly that the protein is involved in forming and maintaining neural synapses, including both neuron-to-neuron synapses and neuron-to-non-neural cell synapses (e.g. neuromotor and neuroendocrine synapses).

Human protein 593 exhibits sequence similarity to HPT (GenBank Accession no. Q92959), as indicated herein in FIGS. 4A through 4E. Protein 593 is a twelve-transmembrane region integral membrane protein having transmembrane regions at approximately the following positions within SEQ ID NO: 7.

1) from about amino acid residue 1 to about residue 10;

2) from about amino-acid residue 33 to about residue 53;

3) from about amino acid residue 62 to about residue 79;

4) from about amino acid residue 118 to about residue 142;

5) from about amino acid residue 153 to about residue 177;

6) from about amino acid residue 200 to about residue 221;

7) from about amino acid residue 262 to about residue 283;

8) from about amino acid residue 314 to about residue 334;

9) from about amino acid residue 347 to about residue 364;

10) from about amino acid residue 469 to about residue 493;

11) from about amino acid residue 509 to about residue 528; and

12) from about amino acid residue 556 to about residue 579.

Extracellular domains are predicted to include approximately amino acid residues 11 to 32, 80 to 117, 178 to 199, 284 to 313, 365 to 468, and 529 to 555 of SEQ ID NO: 7. Intracellular domains are predicted to include approximately amino acid residues 54 to 61, 143 to 152, 222 to 261, 335 to 346, 494 to 508, and 580 to 612 of SEQ ID NO: 7.

Human protein 593 can have additional amino acid residues at the amino terminal end of the sequence listed in SEQ ID NO: 7 (i.e. the protein can have an additional portion at its amino terminus). For example, protein 593 can have 1, 2, 4, 6, 10, 15, 20, 25, or 30 or more additional amino acid residues at the amino terminus indicated in SEQ ID NO: 7.

FIG. 2F depicts a variety of plots produced by computerized analysis of the amino acid sequence of human protein 593. Regions of the protein which are predicted to assume alpha helix (FIG. 2F a)), beta sheet (FIG. 2F c)), turn (FIG. 2F e)), and random coil (FIG. 2F g)) configurations by the Garnier-Robson method are indicated, as are regions predicted to assume alpha helix (FIG. 2F b)), beta sheet (FIG. 2F e)), and turn (FIG. 2F f)) configurations by the Chou-Fasman method. FIG. 2F h) is a Kyte-Doolittle hydrophilicity plot of human protein 593, wherein relatively hydrophilic regions are above the horizontal axis (value=0) and relatively hydrophobic regions are below the horizontal axis. FIG. 2F indications of amphipathic regions of the protein which are predicted to assume alpha (FIG. 2F i)), beta (FIG. 2F j)), and flexible (FIG. 2F k)) configurations by the methods of Eisenberg and Karplus-Schulz, as indicated. FIG. 2F l) and m) are plots of the antigenic index, as calculated by the method of Jameson-Wolf and the surface probability, as calculated by the method of Emini, respectively.

FIG. 2G depicts a hydrophilicity plot of human protein 593. Relatively hydrophobic regions are above the dashed horizontal line, and relatively hydrophilic regions are below the dashed horizontal line. As described elsewhere herein, relatively hydrophilic regions are generally located at or near the surface of a protein, and are more frequently effective immunogenic epitopes than are relatively hydrophobic regions. For example, the region of human protein 593 from about amino acid residue 240 to about amino acid residue 260 appears to be located at or near the surface of the protein, while the region from about amino acid residue 415 to about amino acid residue 430 appears not to be located at or near the surface.

The predicted molecular weight of human protein 593 is about 65.4 kilodaltons.

Biological function of human 593 proteins, nucleic acids encoding them, and modulators of these molecules

Human 593 proteins are involved in disorders which affect both tissues in which they are normally expressed and tissues in which they are normally not expressed. Based on the observation that 593 protein exhibits amino acid sequence homology to human protein 65h2, which is expressed in monkey brain and is therefore likely expressed in human brain tissue, human 593 protein is involved in one or more biological processes which occur in brain and other neurological tissues, although it can also be expressed in other tissues, and involved in disorders in those tissues as well. In particular, 593 is involved in modulating growth, proliferation, survival, differentiation, and activity of cells including, but not limited to, central nervous system neurons, peripheral nervous system neurons, motor neurons, sensory neurons, and sympathetic and parasympathetic neural cells of the animal in which it is normally expressed. Protein 593 is also involved in mediating interactions between cells, particularly between two neurons, or between a neuron and a non-neuronal cell such as a muscle or endocrine cell. Thus, 593 protein has a role in disorders which affect neuronal cells and cells which interact with neurons and their growth, proliferation, survival, differentiation, and activity.

Widespread expression of 593 has been detected among human tissue types. Thus, the growth- , proliferation- , survival- , differentiation- , and activity-modulating activities of 593 protein affect cells of many types. Thus, protein 593 can affect cell-to-cell interactions in a wide variety of cell types.

Protein 593 can also be expressed in other tissues which normally produce or are acted upon by prostaglandins and thromboxanes. Such tissues include, by way of example, blood tissues (e.g. blood platelets), epithelial tissues such as stomach, kidney, lung, uterus, vascular, and other epithelia, liver, ova, and spermatozoa. Protein 593 is thus involved in one or more disorders which affect these tissues, such as one or more of the tissues listed above in the discussion regarding protein 65h2.

The presence of the sugar (or other) transport domain in protein 593 indicates that this protein is involved in transmembrane transport of one or more molecules such as neurotransmitters, prostaglandins, thromboxanes, hormones, small peptides, short polysaccharides (e.g. disaccharides), other charged organic compounds, and the like. The proteins of the invention are therefore involved in one or more disorders relating to inappropriate uptake or release of such molecules (i.e. including inappropriate failure to take up or release such molecules). Protein 593 is thus involved in one or more of a variety of cellular uptake and release disorders such as diabetes, nutritional disorders (e.g. vitamin deficiencies, and-malnutrition), metabolic disorders (e.g. obesity, porphyrias, hyper- and hypolipoproteinemia, lipidoses, and water, electrolyte, mineral, and acid/base imbalances), and neural transmission disorders (e.g. inappropriate pain, dementia, multiple sclerosis, nerve root disorders, Alzheimer's disease, Parkinson's disease, depression physical and psychological substance addiction, sexual dysfunction, schizophrenic disorders, delusional disorders, mood disorders, sleep disorders, and the like).

Occurrence of a Kazal domain in human protein 593 further implicates this protein in neuronal development and neuronal transmission processes. The presence of this domain therefore indicates that 593 protein is involved in disorders relating to inappropriate formation (i.e. including failure to form) and maintenance (i.e. including deterioration) of neuronal synapses, including both neuron-to-neuron synapses and neuron-to-non-neuronal cell synapses. Thus, in addition to the neural transmission disorders described above, protein 593 is also implicated in disorders such as stroke, regeneration of chronically or traumatically damaged neuronal structures (including nerve, brain, and spinal cord), developmental neuronal disorders (e.g. spina bifida), neuronal cancers (e.g. gliomas, astrocytomas, ependymomas, pituitary adenomas, and the like), peripheral nerve deficit, coronary insufficiency, angina, and the like.

The observation that human protein 593 shares sequence homology with proteins involved in transmembrane prostaglandin transport indicates that 593 protein has activity identical or analogous to the activity of those proteins, i.e. that 593 catalyzes or facilitates transmembrane transport of one or more prostaglandins, thromboxanes, other hormones or hormone-like molecules, or other charged organic compounds. Exemplary molecules which can be transported across cell membranes via protein 593 include charged organic compounds, such as one or more of prostaglandins A₁, A₂, B₁, B₂, D₂, E₁, E₂, F_{1α}, F_{2α}, G₂, H₂, I₂, and J₂ and thromboxanes A₂ and B₂. Uptake and release of prostaglandins and thromboxanes, for example, are known to be involved in a variety of physiological processes and disorders including glaucoma, ovum fertilization, sperm motility, pregnancy, labor, delivery, abortion, gastric protection, peptic ulcer formation, intestinal fluid secretion, liver protection, liver damage, liver fibrosis, pain stimulation, glomerular filtration, maintenance of body temperature, fever, airway resistance, asthma, chronic obstructive pulmonary disorder, modulation of blood pressure, hypertension, shock, modulation of inflammation, platelet aggregation, abnormal blood coagulation, atherosclerosis, arteriosclerosis, and coronary artery disease. Thus, polypeptides and nucleic acid molecules of the invention, and compounds which bind with or modulate one or more polypeptides and nucleic acid molecules of the invention can be used to prognosticate, diagnose, inhibit, or treat one or more of the disorders listed above or one or more disorders associated with the physiological processes listed above.

### Protein KIAA0880

A cDNA encoding at least a portion of human KIAA0880 protein was isolated by others from a human brain library of cDNA clones on the basis of the encoded protein being ‘large’ (Nagase et al. (1998) DNA Res. 5:355-364; GenBank submission assigned Accession no. AB020687, submitted Dec. 2, 1998). At the time this cDNA was isolated and submitted to GenBank, it was unknown by the isolators whether the encoded protein had any physiological relevance and, if it did, what that relevance might be. The present inventor has discovered that the protein encoded by the cDNA clone identified by Nagase et al. encodes a transmembrane transport protein that catalyzes transmembrane transport of charged organic compounds such as one or more prostaglandins. In view of this discovery, it is now possible to make use of protein KIAA0880 for the treatment of numerous disorders relating to aberrant transmembrane transport of prostaglandins and/or thromboxanes, and for other purposes.

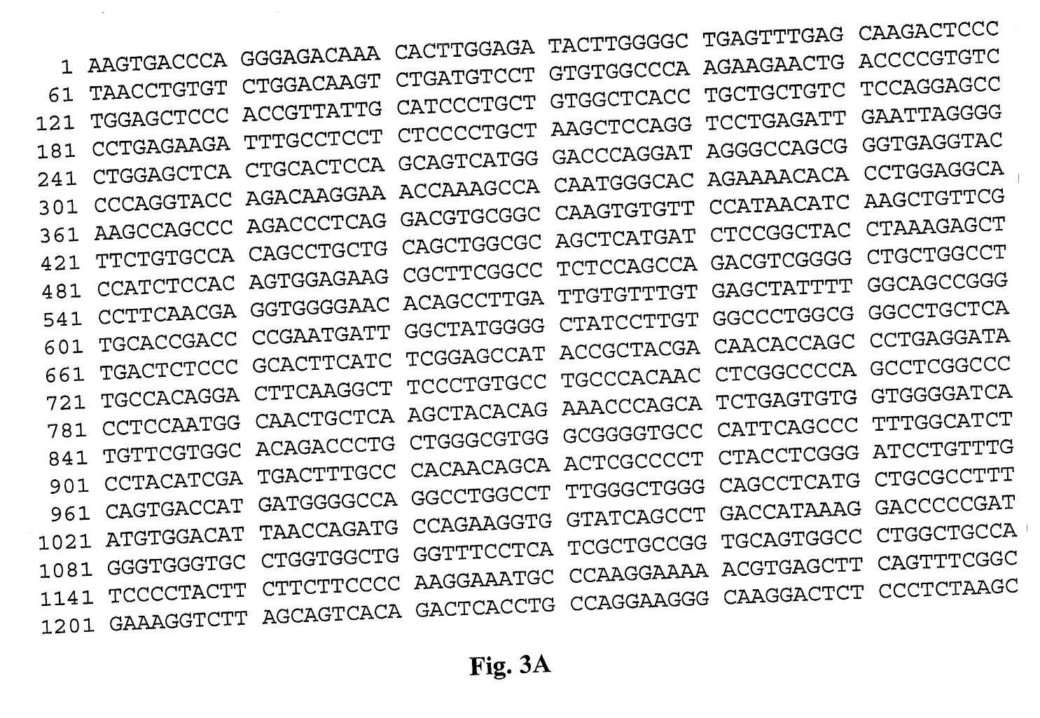

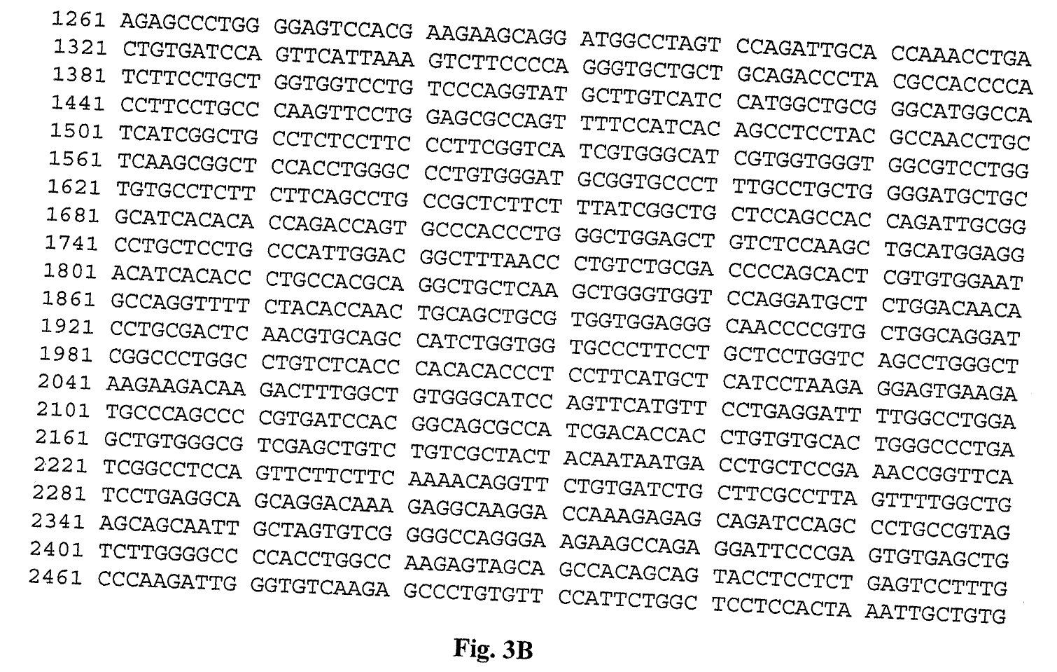

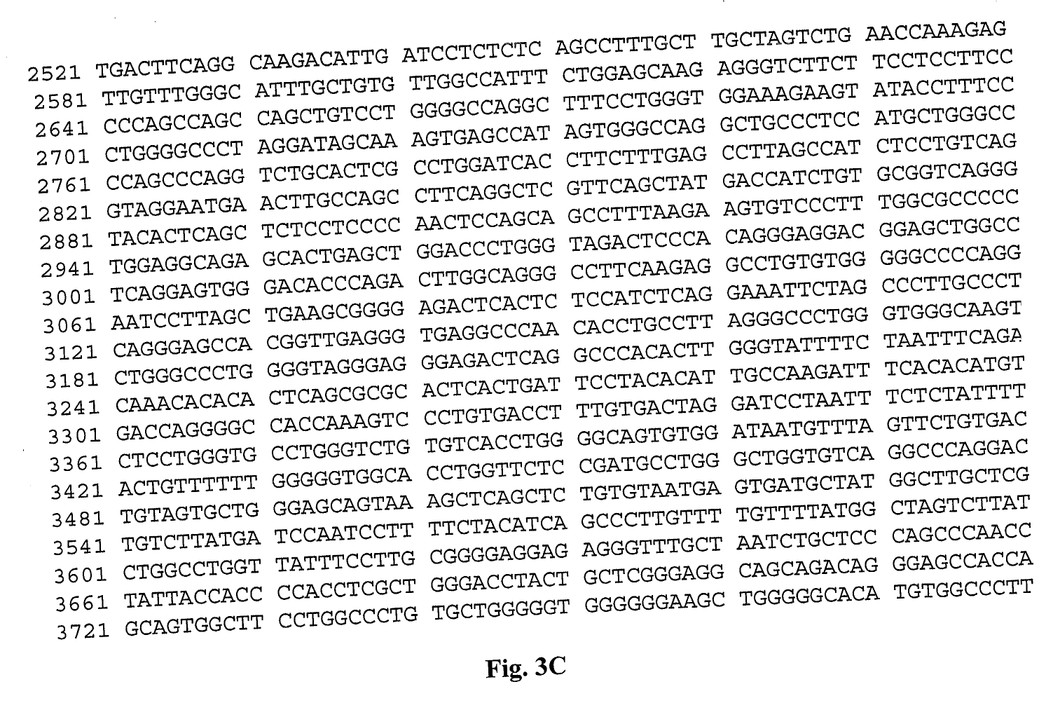

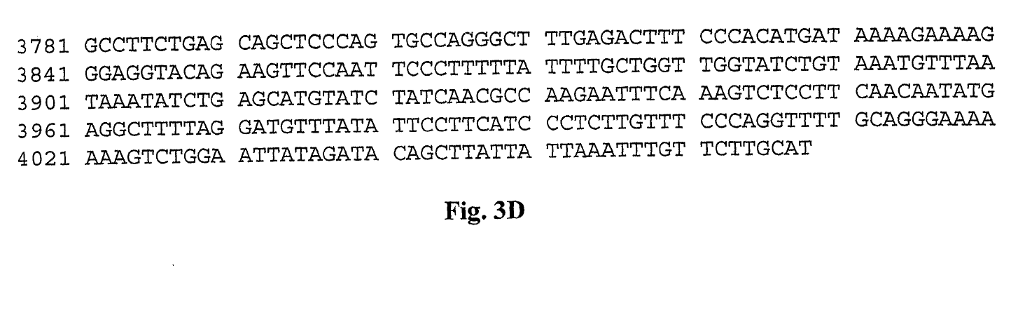

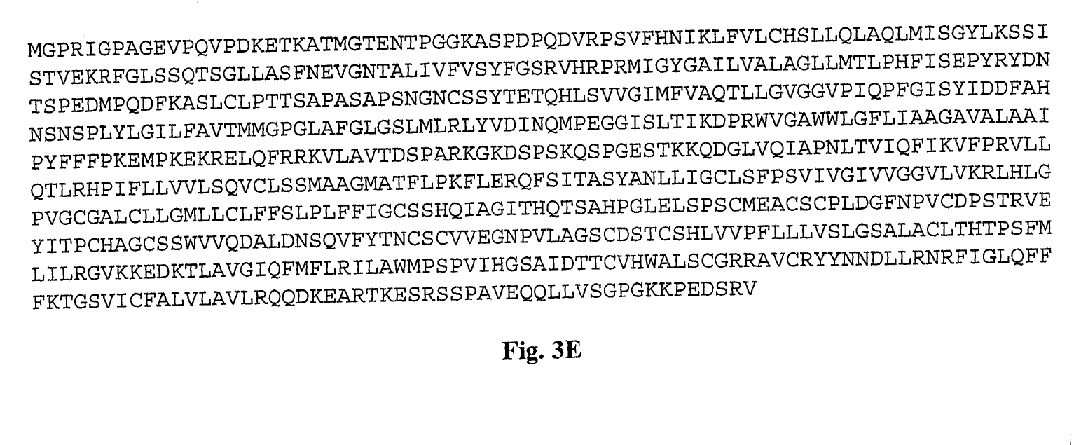

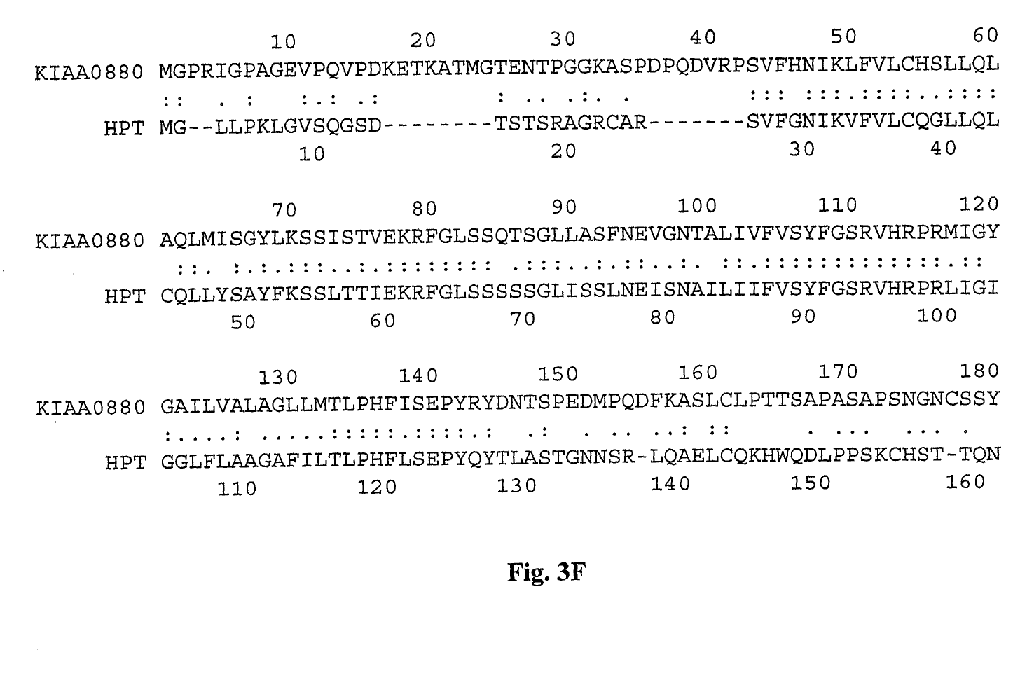

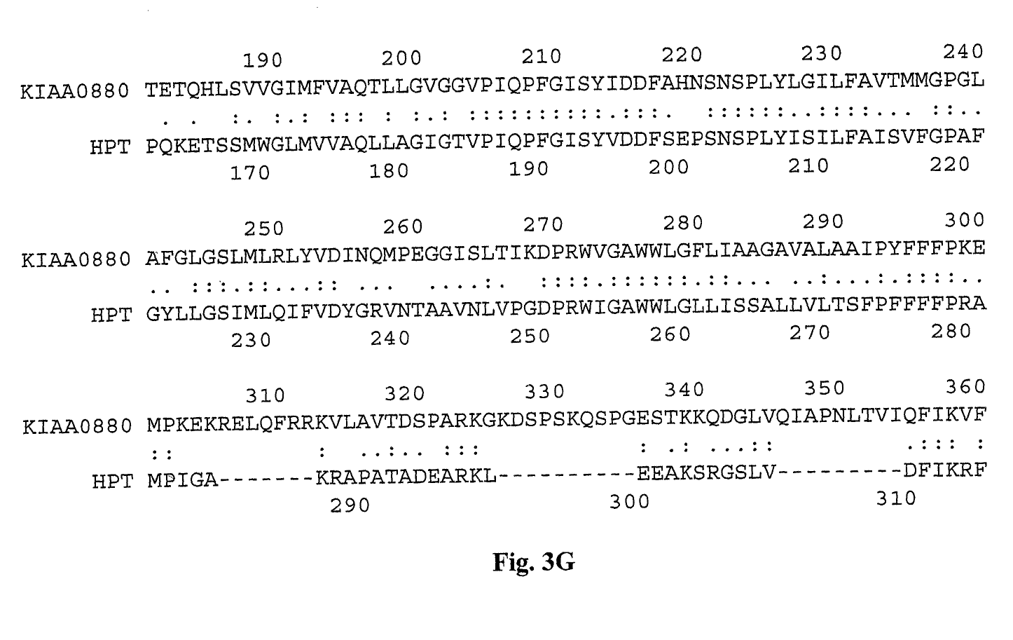

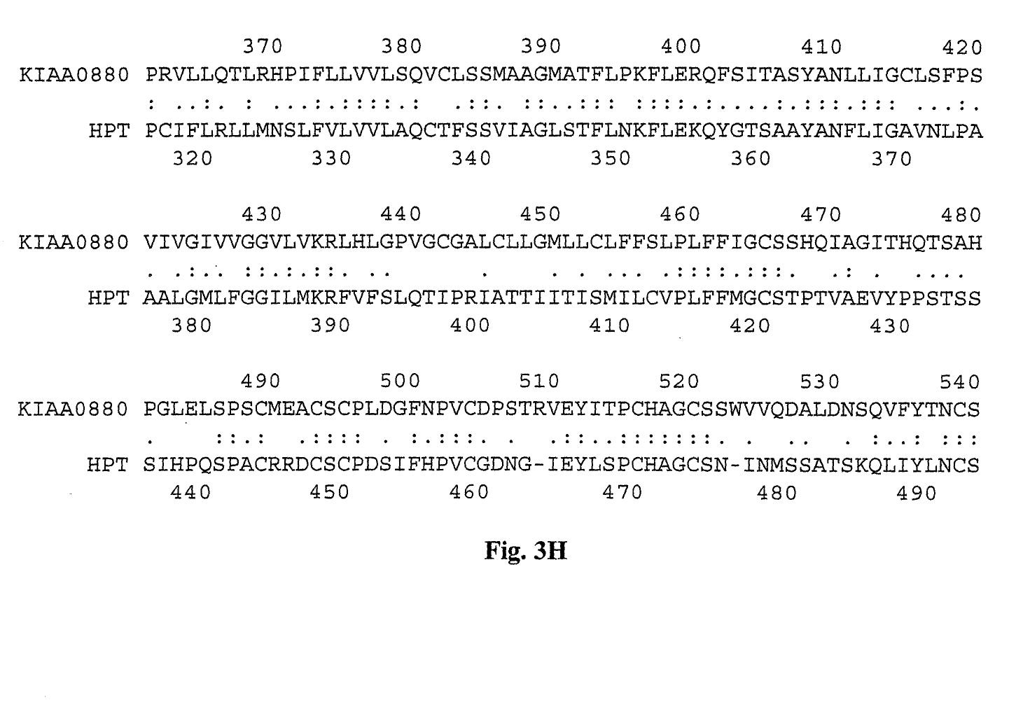

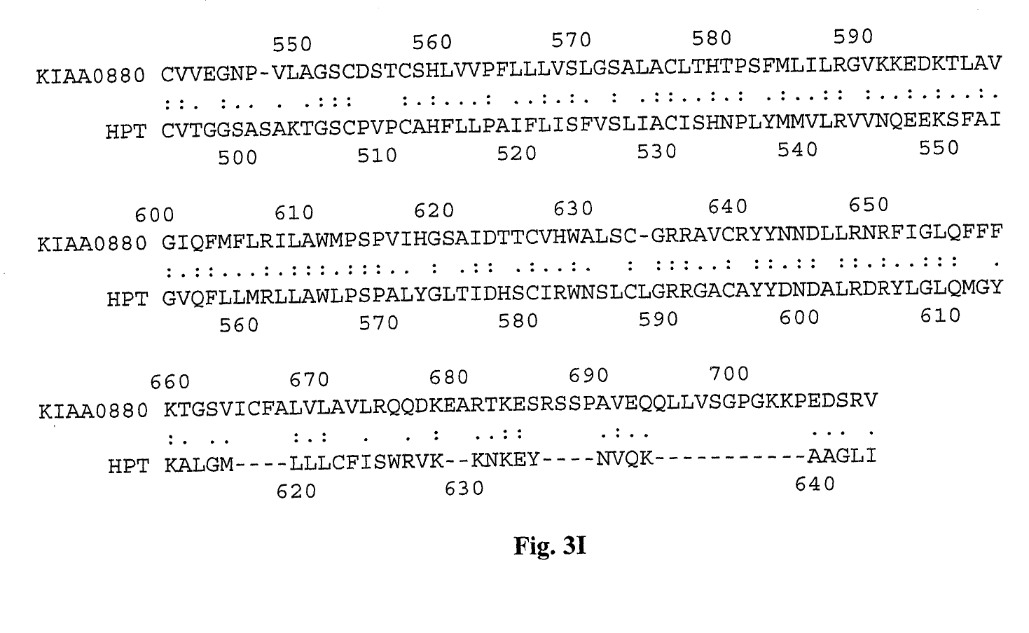

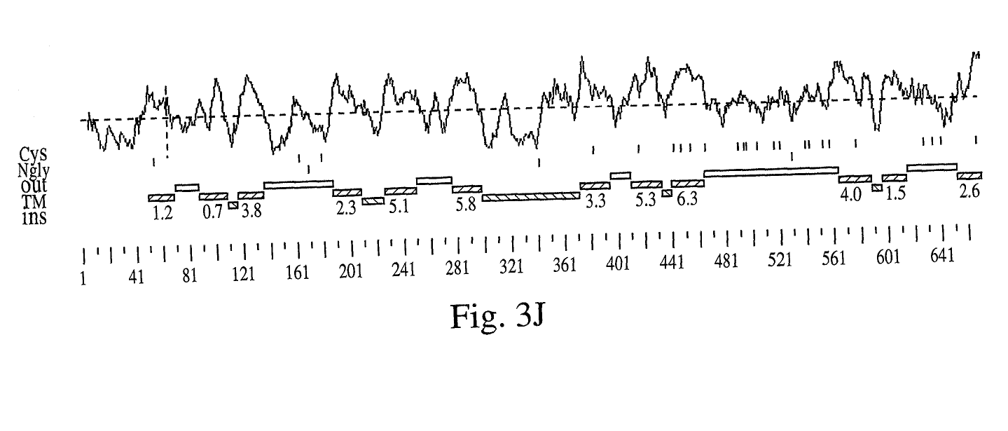

The full length of the cDNA encoding human protein KIAA0880 (FIG. 3; SEQ ID NO: 8) is 4068 nucleotide residues and encodes a 709-amino acid protein (FIG. 3; SEQ ID NO: 9) which exhibits amino acid sequence homology with HPT and other prostaglandin transporters.

KIAA0880 proteins of the invention and nucleic acid molecules encoding them comprise a family of molecules having certain conserved structural and functional features, as indicated in FIGS. 4A through 4E, in which the amino acid sequence of human protein KIAA0880 (SEQ ID NO: 9) is aligned with those of HPT (SEQ ID NO: 10), the human OatP sodium-independent organic anion transporter protein (GenBank Accession no. P46721; SEQ ID-NO: 11), human 65h2 protein (as described herein, SEQ ID NO: 3), and human protein 593 (as described herein, SEQ ID NO: 7).

KIAA0880 proteins typically comprise a variety of potential post-translational modification sites (often within an extracellular domain), such as those described herein in Table V, as predicted by computerized sequence analysis of human KIAA0880 protein using amino acid sequence comparison software (comparing the amino acid sequence of protein KIAA0880 with the information in the PROSITE database {rel. 12.2; Feb, 1995} and the Hidden Markov Models database {Rel. PFAM 3.3 }). In certain embodiments, a protein of the invention has at least 1, 2, 4, 6, 8, or 10 or more of the post-translational modification sites listed in Table V.

TABLE V

| Type of Potential Modification Site | Amino Acid Residues of | Amino Acid |
|---|---|---|
| or Domain | SEQ ID NO:9 | Sequence |
| N-glycosylation site | 176 to 179 | NCSS |
| | 350 to 353 | NLTV |
| | 538 to 541 | NCSC |
| Protein kinase C phosphorylation site | 266 to 268 | TIK |
| | 337 to 339 | STK |
| | 367 to 369 | TLR |
| | 507 to 509 | STR |
| Casein kinase II phosphorylation site | 74 to 77 | STVE |
| | 92 to 95 | SFNE |
| | 147 to 150 | TSPE |
| | 179 to 182 | SYTE |
| | 212 to 215 | SYID |
| | 266 to 269 | TIKD |
| | 333 to 336 | SPGE |
| | 488 to 491 | SCME |
| | 508 to 511 | TRVE |
| | 620 to 623 | SAID |
| N-myristoylation site | 88 to 93 | GLLASF |
| | 129 to 134 | GLLMTL |
| | 175 to 180 | GNCSSY |
| | 228 to 233 | GILFAV |
| | 239 to 244 | GLAFGL |
| | 262 to 267 | GISLTL |
| | 424 to 429 | GIVVGG |
| | 449 to 454 | GMLLCL |
| | 551 to 556 | GSCDST |
| | 571 to 576 | GSALAC |
| | 661 to 666 | GSVICF |
| Amidation site | 633 to 636 | CGRR |
| | 700 to 703 | PGKK |
| Microbodies C-terminal targeting | 707 to 709 | SRV |
| signal | | |

Protein KIAA0880 is predicted by computerized amino acid sequence analysis (using the MEMSAT computer program) to be a twelve-transmembrane region integral membrane protein having transmembrane regions at approximately the following positions within SEQ ID NO: 9.

1) from about amino acid residue 50 to about residue 69;

2) from about amino acid residue 88 to about residue 108;

3) from about amino acid residue 117 to about residue 134;

4) from about amino acid residue 186 to about residue 206;

5) from about amino acid residue 225 to about residue 249;

6) from about amino acid residue 276 to about residue 297;

7) from about amino acid residue 372 to about residue 394;

8) from about amino acid residue 411 to about residue 432;

9) from about amino acid residue 440 to about residue 463;

10) from about amino acid residue 564 to about residue 587;

11) from about amino acid residue 596 to about residue 612; and

12) from about amino acid residue 651 to about residue 673

Extracellular domains are predicted to include approximately amino acid residues 70 to 87, 135 to 185, 250 to 275, 395 to 410, 464 to 563, and 613 to 650 of SEQ ID NO: 9. Intracellular domains are predicted to include approximately amino acid residues 1 to 49, 109 to 116, 207 to 224, 298 to 371, 433 to 439, 588 to 595, and 674 to 709 of SEQ ID NO: 9.

FIG. 3J depicts a hydrophilicity plot of human protein KIAA0880. Relatively hydrophobic regions are above the dashed horizontal line, and relatively hydrophilic regions are below the dashed horizontal line. As described elsewhere herein, relatively hydrophilic regions are generally located at or near the surface of a protein, and are more frequently effective immunogenic epitopes than are relatively hydrophobic regions. For example, the region of human protein KIAA0880 from about amino acid residue 135 to about amino acid residue 155 appears to be located at or near the surface of the protein, while the region from about amino acid residue 160 to about amino acid residue 165 appears not to be located at or near the surface.

Human protein KIAA0880 exhibits sequence similarity to HPT (GenBank Accession no. Q92959), as indicated herein in FIGS. 1I through 1K. FIGS. 3F through 3I depict an alignment of the amino acid sequences of human protein KIAA0880 (SEQ ID NO: 9) and HPT (SEQ ID NO: 11). In this alignment (made using the ALIGN program of the GCG software package, pam120.mat scoring matrix, gap penalties −12/−4), the amino acid sequences of the proteins are 39.5% identical.

The predicted molecular weight of human protein KIAA0880 is about 76.7 kilodaltons.

Biological function of human KIAA0880 proteins, nucleic acids encoding them, and modulators of these molecules

Human KIAA0880 protein is involved in disorders which affect both tissues in which they are normally expressed and tissues in which they are normally not expressed. Based on the observation by others that KIAA0880 protein is expressed in human brain tissue and on the function of this protein as identified herein, human KIAA0880 protein is involved in one or more biological processes which occur in brain and other neurological tissues. In particular, KIAA0880 is involved in modulating growth, proliferation, survival, differentiation, and activity of cells including, but not limited to, central nervous system neurons, peripheral nervous system neurons, motor neurons, sensory neurons, and sympathetic and parasympathetic neural cells of the animal in which it is normally expressed. Protein KIAA0880 is also involved in mediating interactions between cells, particularly between two neurons, or between a neuron and a non-neuronal cell such as a muscle or endocrine cell. Thus, KIAA0880 protein has a role in disorders which affect neuronal cells and cells which interact with neurons and their growth, proliferation, survival, differentiation, and activity.

Widespread expression of KIAA0880 has been detected among human tissue types. Thus, the growth- , proliferation- , survival- , differentiation- , and activity-modulating activities of KIAA0880 protein affect cells of many types. Thus, protein KIAA0880 can affect cell-to-cell interactions in a wide variety of cell types.

Protein KIAA0880 is involved in transmembrane transport of one or more charged organic compounds such as prostaglandins, thromboxanes, and the like. Protein KIAA0880 mediates one or more of facilitated diffusion of the prostaglandin (or thromboxane or the like) and symport or antiport (e.g. involving co-transport of a proton, a sodium ion, a potassium ion, or another physiological ion).

Protein KIAA0880 is therefore involved in transmembrane transport of charged organic molecules such as one or more prostaglandins and thromboxanes in brain and other neural tissues in humans, and is thus involved in, and can be used to prognosticate, prevent, diagnose, or treat, one or more disorders related to inappropriate transmembrane transport (i.e. including inappropriate failure of transport) of prostaglandins, thromboxanes, and the like in neural tissues. Such disorders include, by way of example, neural transmission disorders (e.g. inappropriate pain, dementia, multiple sclerosis, nerve root disorders, Alzheimer's disease, Parkinson's disease, depression, physical and psychological substance addiction, sexual dysfunction, schizophrenic disorders, delusional disorders, mood disorders, sleep disorders, and the like) and disorders relating to inappropriate formation (i.e. including failure to form) and maintenance (i.e. including deterioration) of neuronal synapses, including both neuron-to-neuron synapses and neuron-to-non-neuronal cell synapses. Thus, in addition to the neural transmission disorders described above, protein KIAA0880 is also implicated in, and can be used to prognosticate, prevent, diagnose, or treat, one or more disorders such as stroke, regeneration of chronically or traumatically damaged neuronal structures (including nerve, brain, and spinal cord), developmental neuronal disorders (e.g. spina bifida), neuronal cancers (e.g. gliomas, astrocytomas, ependymomas, pituitary adenomas, and the like), peripheral nerve deficit, coronary insufficiency, angina, and the like. Exemplary molecules which can be transported across cell membranes via protein KIAA0880 include one or more charged organic compounds such as prostaglandins A₁, A₂, B₁, B₂, D₂, E₁, E₂, F_{1α}, F_{2α}, G₂, H₂, I₂, and J₂ and thromboxanes A₂ and B₂. Uptake and release of prostaglandins and thromboxanes, for example, are known to be involved in a variety of physiological processes and disorders including glaucoma, ovum fertilization, sperm motility, pregnancy, labor, delivery, abortion, gastric protection, peptic ulcer formation, intestinal fluid secretion, liver protection, liver damage, liver fibrosis, pain stimulation, glomerular filtration, maintenance of body temperature, fever, airway resistance, asthma, chronic obstructive pulmonary disorder, modulation of blood pressure, hypertension, shock, modulation of inflammation, platelet aggregation, abnormal blood coagulation, atherosclerosis, arteriosclerosis, and coronary artery disease. Thus, polypeptides and nucleic acid molecules of the invention, and compounds which bind with or modulate one or more polypeptides and nucleic acid molecules of the invention can be used to prognosticate, diagnose, inhibit, or treat one or more of the disorders listed above or one or more disorders associated with the physiological processes listed above.

Various aspects of the invention are described in further detail in the following subsections.

I. Isolated Nucleic Acid Molecules

One aspect of the invention pertains to isolated nucleic acid molecules that encode a polypeptide of the invention or a biologically active portion thereof (e.g. a portion encoding the twelve transmembrane portions of one human proteins 65h2, 593, and KIAA0880), as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of all or a portion of SEQ ID NOs: 1, 2, 4, 5, and 6, or a complement thereof, or which has a nucleotide sequence comprising one of these sequences, can be isolated using standard molecular biology techniques and the sequence information provided herein. Using all or a portion of the nucleic acid sequences of SEQ ID NOs: 1, 2, 4, 5, or 6 as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NOs: 1, 2, 4, 5, and 6, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of one of SEQ ID NOs: 1, 2, 4, 5, and 6, or of a naturally occurring mutant of one of SEQ ID NOs: 1, 2, 4, 5, and 6.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of one of SEQ ID NOs: 2 and 6, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of SEQ ID NOs: 1, 2, 4, 5, or 6 due to degeneracy of the genetic code and thus encode the same protein as that encoded by the nucleotide sequence of SEQ ID NOs: 2 or 6.

In addition to the nucleotide sequences of SEQ ID NOs: 2 and 6, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

As used herein, the phrase “allelic variant” refers to a nucleotide sequence which occurs at a given locus or to a polypeptide encoded by the nucleotide sequence. For example, as described herein, the gene encoding human protein 65h2 maps to chromosome 15 at q26.1. Allelic variants of this gene therefore map to chromosome 15 at q26.1 and have individual nucleotide sequences that are highly homologous with the naturally-occurring 65h2 gene.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the human proteins described herein are intended to be within the scope of the invention. Nucleic acid molecules corresponding to natural allelic variants and homologs of a cDNA of the invention can be isolated based on their identity to human nucleic acid molecules using the human 65h2, 593, or KIAA0880 cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, 5000, 10000, 20000, 40000, or 80000 or more) nucleotides in length and hybridizes under stringent conditions to the nucleic acid molecule comprising the nucleotide sequence, preferably the coding sequence, of SEQ ID NOs: 1, 2, 4, 5, or 6, or a complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NOs: 1, 2 or 6, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that can exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may-be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes which alter amino acid residues that are not essential for activity. Such polypeptides differ in amino acid sequence from SEQ ID NOs: 3 and 7, and yet retain biological activity. In one embodiment, the isolated nucleic acid molecule has a nucleotide sequence encoding a protein that includes an amino acid sequence that is at least about 40% identical, 50%, 60%, 70%, 80%, 90%, 95%, or 98% identical to the amino acid sequence of one of SEQ ID NOs: 3 and 7.

An isolated nucleic acid molecule encoding a variant protein can be created by introducing one or more nucleotide substitutions, additions, or deletions into the nucleotide sequence of SEQ ID NOs: 1, 2, 4, 5, or 6, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), non-charged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein: protein interactions with the polypeptide of the invention; (2) the ability to bind a ligand of the polypeptide of the invention (e.g. another protein identified herein); (3) the ability to bind with a modulator or substrate of the polypeptide of the invention; or (4) the ability to modulate a physiological activity of the protein, such as one of those disclosed herein.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ non-translated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using-procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, as described further in the following subsection).

The antisense nucleic acid molecules of the invention are typically administered to a subject or generated in situ such that they hybridize with or bind with cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds with DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind with receptors or antigens expressed on a selected cell surface, e.g., by linking the antisense nucleic acid molecules to peptides or antibodies which bind with cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved as described in U.S. Pat. No. 4,987,071 and U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength The synthesis of PNA oligomers can be performed using standard solid phase peptide synthesis protocols as described (Hyrup et al. (1996), supra; Perry-O'Keefe et al. (1996) Proc. Natl. Acad. Sci USA 93: 14670-675).

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a step-wise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci USA 86:6553-6556; Lemaitre et al. (1987) Proc. Natl. Acad. Sci. USA 84:648-652; PCT Publication No. WO 88/09810) or the blood-brain barrier (see, e.g, PCT Publication No. WO 89/10134). In addition, oligonucleotides can be modified with hybridization-triggered cleavage agents (see, e.g., Krol et at. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

II. Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. As an alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in either of SEQ ID NOs: 3 and 7), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of one of SEQ ID NOs: 3 and 7. Other useful proteins are substantially identical (e.g., at least about 40%, preferably 50%, 60%, 70%, 80%, 90%, 95%, or 99%) to either of SEQ ID NOs: 3 and 7 and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., percent identity is equal to the number of identical positions divided by the total number of positions (e.g., overlapping positions) multiplied by 100). In one embodiment the two sequences are the same length.

The determination of percent identity between two sequences can be accomplished using a mathematical algorithm A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably a biologically active part) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which the polypeptide of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et at., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding a prostaglandin or a thromboxane and inhibiting transmembrane transport thereof. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477). Methods for assessing transmembrane transport of compounds such as prostaglandins and thromboxanes are described elsewhere herein.

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of SEQ ID NOs: 3 and 7, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. FIGS. 1N and 2G are hydrophobicity plots of the proteins of the invention. These plots or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds with a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non human monoclonal antibody, e.g., a murine antibody, is used to guide the selection of a completely human antibody recognizing the same epitope (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast, or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g, Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram).

As defined herein, a therapeutically effective amount of protein or polypeptide (i.e., an effective dosage) ranges from about 0.001 to 30 milligrams per kilogram body weight, preferably about 0.01 to 25 milligrams per kilogram body weight, more preferably about 0.1 to 20 milligrams per kilogram body weight, and even more preferably about 1 to 10 milligrams per kilogram, 2 to 9 milligrams per kilogram, 3 to 8 milligrams per kilogram, 4 to 7 milligrams per kilogram, or 5 to 6 milligrams per kilogram body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with an antibody, protein, or polypeptide in the range of from about 0.1 to 20 milligrams per kilogram body weight, one time per week for about 1 to 10 weeks, preferably for about 2 to 8 weeks, more preferably for about 3 to 7 weeks, and even more preferably for about 4, 5, or 6 weeks. It will also be appreciated that the effective dosage of the antibody, protein, or polypeptide used for treatment may increase or decrease over the course of a particular treatment. Changes in dosage may result and become apparent from the results of diagnostic assays as described herein.

It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is about 0.1 mg/kg to 100 mg/kg of body weight (generally about 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et at. ((1997) J. Acquired Immune Deficiency Syndromes and Human Retrovirology 14:193).

Further, an antibody (or fragment thereof) may be conjugated to a therapeutic moiety such as a cytotoxin, a therapeutic agent, or a radioactive metal ion. A cytotoxin or cytotoxic agent can be substantially any agent that is detrimental to a cell when it is provided to the cell. Exemplary cytotoxins include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carnustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other therapeutic moieties which can be conjugated with antibodies include proteins and polypeptides possessing a desirable biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

Instead of conjugating the therapeutic moiety with the antibody, the therapeutic moiety may alternatively be co-administered with the antibody. Co-administration can be simultaneous (e.g. administration of a single composition containing both the antibody and the therapeutic moiety or administration of distinct compositions, at least one of which contains the antibody and at least another of which contains the therapeutic moiety), or overlapping. Overlapping co-administration refers to separate administration of the therapeutic moiety and the antibody to the same subject, wherein the separate administrations are sufficiently close in time that the therapeutic moiety and the antibody are simultaneously present in the body of the subject. For example, a therapeutic moiety which, when orally administered, does not appear in the blood stream in significant quantities for one hour can be administered to a subject about one hour prior to infusion of an antibody into the bloodstream of the subject, so that the therapeutic moiety and the antibody co-exist in the bloodstream.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologs, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used for all of the purposes identified herein in portions of the disclosure relating to individual types of protein of the invention (e.g. 65h2 proteins and 593 proteins), as can human protein KIAA0880 and fragments, derivatives, and allelic variants thereof (“KIAA0880-related polypeptides”). The isolated nucleic acid molecules of the invention and nucleic acids encoding KIAA0880-related polypeptides can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention and KIAA0880-related polypeptides can be used to screen drugs or compounds which modulate activity or expression of the polypeptide as well as to treat disorders characterized by insufficient or excessive production of a protein of the invention (or KIAA0880) or production of a form of the protein which has decreased or aberrant activity compared to the wild type protein. In addition, the antibodies of the invention can be used to detect and isolate a protein of the and modulate activity of a protein of the invention or of a KIAA0880-related polypeptide.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind with a polypeptide of the invention or to a KIAA0880-related polypeptide, or have a stimulatory or inhibitory effect on, for example, expression or activity of a polypeptide of the invention or of a KIAA0880-related polypeptide.

In one embodiment, the invention provides assays for screening candidate or test compounds which bind with or modulate the activity of the membrane-bound form of a polypeptide of the invention, a KIAA0880-related polypeptide, or a biologically active portion of one of these. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is generally limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckennann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, a KIAA0880-related polypeptide, or a biologically active portion of one of these, on the cell surface is contacted with a test compound and the ability of the test compound to bind with the polypeptide determined. The cell, for example, can be a yeast cell or a cell of mammalian origin. Determining the ability of the test compound to bind with the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, a membrane-bound form of a KIAA0880-related polypeptide, or a biologically active portion of one of these, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind with the polypeptide or a biologically active portion thereof as compared to the known compound.

In another embodiment, the assay involves assessment of an activity characteristic of a polypeptide of the invention or of a KIAA0880-related polypeptide, wherein binding of the test compound with the polypeptide or a biologically active portion thereof alters (i.e. increases or decreases) the activity of the polypeptide. For example, the method described in U.S. Pat. No. 5,792,851 for evaluating uptake of a prostaglandin by a cell expressing a nucleic acid encoding a prostaglandin transporter may be used to assess prostaglandin or thromboxane uptake by a cell expressing a nucleic acid encoding a nucleic acid of the invention. In this assay, a test cell which expresses a nucleic acid encoding a polypeptide of the invention or a KIAA0880-related polypeptide is contacted with a fluid containing a labeled (e.g. tritiated) prostaglandin or thromboxane, and uptake of the labeled compound into the cell is assessed over time by isolating the test cells from the fluid and assessing the amount of label associated with the cells. For example, cultured HeLa cells can be transfected with a recombinant Vaccinia virus vector comprising a nucleic acid encoding a polypeptide of the invention or a KIAA0880-related polypeptide. A tritiated prostaglandin or thromboxane is added to the medium, and the medium containing the labeled compound is rinsed from the cells after a selected amount of time. The tritium content of the cells (i.e. corresponding to prostaglandin/thromboxane uptake by the cells) is assessed with, for example, a scintillation counter. The skilled artisan will understand how this assay can be modified to accommodate particular test cells, nucleic acid vectors, and particular prostaglandins or thromboxanes.

In another embodiment, an assay is a cell-based assay comprising contacting a cell expressing a membrane-bound form of a polypeptide of the invention, a membrane-bound form of a KIAA0880-related polypeptide, or a biologically active portion of one of these, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind with or interact with a target molecule or to transport molecules across the cytoplasmic membrane.

Determining the ability of a polypeptide of the invention, or of a KIAA0880-related polypeptide, to bind with or interact with a target molecule can be accomplished by one of the methods described above for determining direct binding. As used herein, a “target molecule” is a molecule with which a selected polypeptide (e.g., a polypeptide of the invention or a KIAA0880-related polypeptide) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention, a KIAA0880-related polypeptide, or some other polypeptide or protein. For example, a target molecule can be a component of a signal transduction pathway which facilitates transduction of an extracellular signal (e.g., a signal generated by binding of a compound to a polypeptide of the invention or to a KIAA0880-related polypeptide) through the cell membrane and into the cell or a second intercellular protein which has catalytic activity or a protein which facilitates the association of downstream signaling molecules with a polypeptide of the invention. Determining the ability of a polypeptide of the invention to bind with or interact with a target molecule can be accomplished by determining the activity of the target molecule. For example, the activity of the target molecule can be determined by detecting induction of a cellular second messenger of the target (e.g., an mRNA, intracellular Ca²⁺, diacylglycerol, IP3, and the like), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a polypeptide of the invention, a KIAA0880-related polypeptide, or a biologically active portion of one of these with a test compound and determining the ability of the test compound to bind with the polypeptide or biologically active portion thereof. Binding of the test compound to the polypeptide can be determined either directly or indirectly as described above. In a preferred embodiment, the assay includes contacting the polypeptide of the invention, the KIAA0880-related polypeptide, or the biologically active portion with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind with the polypeptide or biologically active portion thereof as compared to the known compound.

In another embodiment, an assay is a cell-free assay comprising contacting a polypeptide of the invention, a KIAA0880-related polypeptide, or a biologically active portion of one of these with a test compound and determining the ability of the test compound to modulate (e.g., stimulate or inhibit) the activity of the polypeptide or biologically active portion. It can be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

Determining the ability of the test compound to modulate the activity of the polypeptide can be accomplished, for example, by determining the ability of the polypeptide to bind with a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic activity, the enzymatic activity, or both, of the target molecule on an appropriate substrate can be determined as previously described.

In yet another embodiment, the cell-free assay comprises contacting a polypeptide of the invention, a KIAA0880-related polypeptide, or a biologically active portion of one of these with a known compound which binds the polypeptide to form an assay mixture, contacting the assay mixture with a test compound; and determining the ability of the test compound to interact with the polypeptide, wherein determining the ability of the test compound to interact with the polypeptide comprises determining the ability of the polypeptide to preferentially bind with or modulate the activity of a target molecule.

In one or more embodiments of the above assay methods of the present invention, it can be desirable to immobilize either a polypeptide of the invention, a KIAA0880-related polypeptide, or a target molecule of one of these in order to facilitate separation of complexed from non-complexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione Sepharose beads (Sigma Chemical; St. Louis, Mo.) or glutathione derivatized microtiter plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtiter plate wells are washed to remove any non-bound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, a polypeptide of the invention, a KIAA0880-related polypeptide, or a target molecule of one of these can be immobilized utilizing conjugation of biotin and streptavidin. Biotinylated polypeptides or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with polypeptides or target molecules but which do not interfere with binding of the polypeptides to its target molecule can be derivatized to the wells of the plate, and non-bound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention or a KIAA0880-related polypeptide are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention or a KIAA0880-related polypeptide based on this comparison. For example, when expression of the selected mRNA or protein is greater (i.e. statistically significantly greater) in the presence of the candidate compound than in its absence, the candidate compound is identified as a stimulator of the selected mRNA or protein expression. Alternatively, when expression of the selected mRNA or protein is less (i.e. statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the invention or a KIAA0880-related polypeptide can be used as a “bait protein” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind with or interact with the polypeptide of the invention or KIAA0880-related polypeptide and modulate activity of the polypeptide. Such binding proteins are also likely to be involved in the propagation of signals by the polypeptide as, for example, upstream or downstream elements of a signaling pathway involving the polypeptide.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sub-localization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al. (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and not affected with a disease associated with a gene of the invention can be determined. If a mutation is observed in some or all of the affected individuals but not in any non-affected individuals, then the mutation is likely to be the causative agent of the particular disease. Comparison of affected and non-affected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individuals DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of SEQ ID NOs: 1, 4, and 5 can comfortably provide positive individual identification with a panel of perhaps 10 to 1,000 primers which each yield a non-coding amplified sequence of 100 bases. If predicted coding sequences, such as those in SEQ ID NOs: 2 and 6 are used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions are particularly appropriate for this use as greater numbers of polymorphisms occur in the non-coding regions, making it easier to differentiate individuals using this technique. Examples of polynucleotide reagents include the nucleic acid sequences of the invention or portions thereof, e.g., fragments derived from non-coding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention or of a KIAA0880-related polypeptide, in the context of a biological sample (e.g., blood, serum, cells, tissue) to thereby determine whether an individual is afflicted with a disease or disorder, or is at risk of developing a disorder, associated with aberrant expression or activity of a polypeptide of the invention or aberrant expression of a KIAA0880-related polypeptide. The invention also provides for prognostic (or predictive) assays for determining whether an individual is at risk of developing a disorder associated with aberrant expression or activity of a polypeptide of the invention or aberrant expression of a KIAA0880-related polypeptide. For example, mutations in a gene of the invention or in a gene encoding KIAA0880 can be assayed in a biological sample. Such assays can be used for prognostic or predictive purpose to thereby prophylactically treat an individual prior to the onset of a disorder characterized by or associated with aberrant expression or activity of a polypeptide of the invention or a KIAA0880-related polypeptide.

Another aspect of the invention provides methods for assessing expression of a nucleic acid or polypeptide of the invention or of a KIAA0880-related polypeptide or a nucleic acid encoding it, and for assessing activity of a polypeptide of the invention or a KIAA0880-related polypeptide in an individual to thereby select appropriate therapeutic or prophylactic agents for that individual (referred to herein as “pharmacogenomics”). Pharmacogenomics allows selection of agents (e.g., drugs) for therapeutic or prophylactic treatment of an individual based on the genotype of the individual (e.g., the genotype of the individual is examined to determine the ability of the individual to respond to a particular agent).

Yet another aspect of the invention pertains to monitoring the influence of agents (e.g., drugs or other compounds) on the expression or activity of a polypeptide of the invention or a KIAA0880-related polypeptide in clinical trials. These and other agents are described in further detail in the following sections.

1 Diagnostic Assays

An exemplary method for detecting the presence or absence of a polypeptide or nucleic acid of the invention, or of a KIAA0880-related polypeptide or a nucleic acid encoding it, in a biological sample involves obtaining a biological sample from a test subject and contacting the biological sample with a compound or an agent capable of detecting the polypeptide or nucleic acid (e.g., mRNA, genomic DNA) such that the presence of the polypeptide or nucleic acid is detected in the biological sample. A preferred agent for detecting mRNA or genomic DNA encoding a polypeptide of the invention, or a KIAA0880-related polypeptide, is a labeled nucleic acid probe capable of hybridizing to mRNA or genomic DNA encoding the polypeptide. The nucleic acid probe can be, for example, a full-length cDNA, such as the nucleic acid of SEQ ID NOs: 1, 5, or 8 or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention or of a KIAA0880-related polypeptide. Other suitable probes for use in the diagnostic assays of the invention are described herein.

A preferred agent for detecting a polypeptide of the invention, or of a KIAA0880-related polypeptide, is an antibody capable of binding to the polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention, or a KIAA0880-related polypeptide, include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention, or of a KIAA0880-related polypeptide, include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a tissue (e.g. a neuronal tissue) sample isolated by conventional means from a subject.

In another embodiment, the methods further involve obtaining a control biological sample from a control subject, contacting the control sample with a compound or agent capable of detecting a polypeptide of the invention, an mRNA or genomic DNA encoding a polypeptide of the invention, a KIAA0880-related polypeptide, or an mRNA or genomic DNA encoding a KIAA0880-related polypeptide, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention, or of a KIAA0880-related polypeptide or a nucleic acid encoding it, in a biological sample (a test sample). Such kits can be used to determine if a subject is suffering from or is at increased risk of developing a disorder associated with aberrant expression of a polypeptide of the invention or with aberrant expression of a KIAA0880-related polypeptide (e.g., one of the disorders described in the section of this disclosure wherein the individual polypeptide of the invention is discussed). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds with DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds with a polypeptide of the invention or to a KIAA0880-related polypeptide; and, optionally, (2) a second, different antibody which binds with either the polypeptide or the first antibody and is conjugated to a detectable agent.

For oligonucleotide-based kits, the kit can comprise, for example: (1) an oligonucleotide, e.g., a detectably labeled oligonucleotide, which hybridizes with a nucleic acid encoding a polypeptide of the invention or with a nucleic acid encoding a KIAA0880-related polypeptide or (2) a pair of primers useful for amplifying a nucleic acid encoding a polypeptide of the invention or a KIAA0880-related polypeptide. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package along with instructions for observing whether the tested subject is suffering from or is at risk of developing a disorder associated with aberrant expression of the polypeptide.

2. Prognostic Assays

The methods described herein can furthermore be utilized as diagnostic or prognostic assays to identify subjects having or at risk of developing a disease or disorder associated with aberrant expression or activity of a polypeptide of the invention or with aberrant expression or activity of a KIAA0880-related polypeptide. For example, the assays described herein, such as the preceding diagnostic assays or the following assays, can be utilized to identify a subject having or at risk of developing a disorder associated with aberrant expression or activity of a polypeptide of the invention or with aberrant expression or activity of a KIAA0880-related polypeptide (e.g., one of the disorders described in the section of this disclosure wherein the individual polypeptides of the invention are discussed). Alternatively, the prognostic assays can be utilized to identify a subject afflicted with or at risk for developing such a disease or disorder. Thus, the present invention provides a method in which a test sample is obtained from a subject and a polypeptide or nucleic acid (e.g., mRNA, genomic DNA) of the invention is detected, or a KIAA0880-related polypeptide or a nucleic acid encoding it, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention or with aberrant expression or activity of a KIAA0880-related polypeptide. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention, or of a KIAA0880-related polypeptide, in which a test sample is obtained and the polypeptide, or nucleic acid encoding the polypeptide, is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized by aberrant expression or activity of a polypeptide of the invention or by aberrant expression or activity of a KIAA0880-related polypeptide. In preferred embodiments, the methods include detecting, in a sample of cells obtained from the subject, the presence or absence of a genetic lesion or mutation characterized by at least one of an alteration affecting the integrity of a gene encoding the polypeptide of the invention, an alteration affecting the integrity of a gene encoding a KIAA0880-related polypeptide, mis-expression of a gene encoding a polypeptide of the invention, and mis-expression of a gene encoding a KIAA0880-related polypeptide. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting mutations described herein.

Alternative amplification methods include: self-sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101;

Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. RNA/DNA duplexes can be treated with RNASE to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp’ of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization; Gibbs et al. (1989) Nucleic Acids Res. 17:2437-2448) or at the extreme 3′ end of one primer where, under appropriate conditions, mismatching can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention or a KIAA0880-related polypeptide. Furthermore, any cell type or tissue (e.g. a neuronal tissue) in which a polypeptide of the invention, or a KIAA0880-related polypeptide, is expressed can be utilized in the prognostic assays described herein.

3. Pharmacogenomics

Agents, or modulators which have a stimulatory or inhibitory effect on activity or expression of a polypeptide of the invention, or on activity or expression of a KIAA0880-related polypeptide, as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, mutation content of a gene of the invention, activity of a KIAA0880-related polypeptide, expression of a nucleic acid encoding a KIAA0880-related polypeptide, or mutation content of a gene encoding a KIAA0880-related polypeptide in an individual can be determined to thereby select appropriate agent(s) for therapeutic or prophylactic treatment of the individual.

The field of pharmacogenomics deals with clinically significant hereditary variations in the response to drugs due to altered drug disposition and abnormal action in affected persons. See, e.g, Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, mutation content of a gene encoding the polypeptide, activity of a KIAA0880-related polypeptide, expression of a nucleic acid encoding a KIAA0880-related polypeptide, or mutation content of a gene encoding a KIAA0880-related polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

4. Monitoring of Effects During Clinical Trials

Monitoring the influence of agents (e.g., drug compounds) on the expression or activity of a polypeptide of the invention, or of a KIAA0880-related polypeptide, (e.g., the ability to modulate transmembrane transport of a charged organic compounds such as a prostaglandin or thromboxane) can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity.

For example, and not by way of limitation, genes, including those encoding a polypeptide of the invention and those encoding a KIAA0880-related polypeptide, that are modulated in cells by treatment with an agent (e.g., compound, drug or small molecule) which modulates activity or expression of the polypeptide (e.g., as identified in a screening assay described herein) can be identified. Thus, to study the effect of agents on disorders relating to aberrant prostaglandin uptake, for example, in a clinical trial, cells can be isolated and RNA prepared and analyzed for the levels of expression of a gene encoding a polypeptide of the invention, of a gene encoding a KIAA0880-related polypeptide, or of another gene implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of a polypeptide or nucleic acid of the invention, or of a KIAA0880-related polypeptide or a nucleic acid encoding such a polypeptide, in the post-administration samples; (v) comparing the level of the polypeptide or nucleic acid in the pre-administration sample with the level of the polypeptide or nucleic acid in the post-administration sample or samples; and (vi) altering the administration of the agent to the subject accordingly. For example, increased administration of the agent can be desirable to increase the expression or activity of the polypeptide or nucleic acid to higher levels than detected, i.e., to increase the effectiveness of the agent. Alternatively, decreased administration of the agent can be desirable to decrease expression or activity of the polypeptide or nucleic acid to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant expression or activity of a polypeptide of the invention or of a KIAA0880-related polypeptide and/or in which the polypeptide of the invention or a KIAA0880-related polypeptide is involved. Such disorders are described elsewhere in this disclosure.

1. Prophylactic Methods

In one aspect, the invention provides a method for preventing in a subject, a disease or condition associated with an aberrant expression or activity of a polypeptide of the invention, or of a KIAA0880-related polypeptide, by administering to the subject an agent which modulates expression or at least one activity of the polypeptide. Subjects at risk for a disease which is caused or contributed to by aberrant expression or activity of a polypeptide can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

2. Therapeutic Methods

Another aspect of the invention pertains to methods of modulating expression or activity of a polypeptide of the invention, or of a KIAA0880-related polypeptide, for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include a polypeptide of the invention, a nucleic acid encoding the polypeptide of the invention, a KIAA0880-related polypeptide, and a nucleic acid encoding the KIAA0880-related polypeptide that has been introduced into the cell. In another embodiment, the agent inhibits one or more of the biological activities of a polypeptide of the invention or of a KIAA0880-related polypeptide. Examples of such inhibitory agents include antisense nucleic acid molecules and antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a polypeptide of the invention or of a KIAA0880-related polypeptide. In one embodiment, the method involves administering an agent (e.g., an agent identified by a screening assay described herein), or combination of agents that modulates (e.g., up-regulates or down-regulates) expression or activity. In another embodiment, the method involves administering a polypeptide of the invention, a nucleic acid of the invention, a KIAA0880-related polypeptide, or a nucleic acid encoding a KIAA0880-related polypeptide, as therapy to compensate for reduced or aberrant expression or activity of the polypeptide.

Stimulation of activity is desirable in situations in which activity or expression is abnormally low or down-regulated and/or in which increased activity is likely to have a beneficial effect, e.g., in wound healing. Conversely, inhibition of activity is desirable in situations in which activity or expression is abnormally high or up-regulated and/or in which decreased activity is likely to have a beneficial effect.

The contents of all references, patents, and published patent applications cited throughout this application are hereby incorporated by reference.

Biological Deposit

Clones encoding human 65h2 and 593 proteins were deposited with ATCC on Jul. 22, 1999 in the form of a mixture of two plasmids, one (Ep65h2) encoding protein 65h2, the other (Ep593) encoding protein 593. This deposit will be maintained under the terms of the Budapest Treaty on the International Recognition of the Deposit of Microorganisms for the Purposes of Patent Procedure.

In order to check for the presence of Ep65h2 and Ep593 in the deposited mixture, an E. coli host strain (e.g. DH5α) is transformed using the mixture and plated and incubated on Luria broth plates containing 100 micrograms per milliliter ampicillin. About 10 to 20 transformants are selected and subjected to a standard plasmid minipreparation procedure. Each DNA is digested using restriction endonuclease EcoRI and the fragments are separated by, for example, agarose gel electrophoresis. Fragments are visualized (e.g. using ethidium bromide in the agarose gel). EcoRI digestion of Ep62h5 yields one band approximately 5.5 kB in size. EcoRI digestion of Ep62h5 yields two bands, one having a size of about 3.5 kB, and the other having a size of about 1.5 kB.

This deposit was made merely as a convenience to those of skill in the art. This deposit is not an admission that a deposit is required pursuant to 35 U.S.C. §112.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 40% identical to the nucleotide sequence of SEQ ID NOs: 1, 2, 4, 5, and 6, or a complement thereof;
b) a nucleic acid molecule comprising at least 404 nucleotide residues and having a nucleotide sequence identical to at least 404 consecutive nucleotide residues of SEQ ID NOs: 1, 2, or 4, or a complement thereof;
c) a nucleic acid molecule comprising at least 434 nucleotide residues and having a nucleotide sequence identical to at least 434 consecutive nucleotide residues of SEQ ID NOs: 5 or 6, or a complement thereof;
d) a nucleic acid molecule which encodes a polypeptide comprising the amino acid sequence of SEQ ID NOs: 3 or 7, or a complement thereof;
e) a nucleic acid molecule which encodes a fragment of a polypeptide comprising the amino acid sequence of SEQ ID NOs: 3 or 7, wherein the fragment comprises at least 10 consecutive amino acid residues of SEQ ID NO: 3 or at least 11 consecutive amino acid residues of SEQ ID NO: 7; and
f) a nucleic acid molecule which encodes a naturally occurring allelic variant of a polypeptide comprising the amino acid sequence of SEQ ID NOs: 3 or 7, wherein the nucleic acid molecule hybridizes to a nucleic acid molecule consisting of the nucleotide sequence of SEQ ID NOs: 1, 2, 4, 5, and 6, or a complement thereof under stringent conditions.

2. The isolated nucleic acid molecule of claim 1, which is selected from the group consisting of:
a) a nucleic acid having the nucleotide sequence of SEQ ID NOs: 1, 2, 4, 5, and 6, or a complement thereof; and
b) a nucleic acid molecule which encodes a polypeptide having the amino acid sequence of SEQ ID NOs: 3 or 7, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NOs: 3 or 7, wherein the fragment comprises at least 10 contiguous amino acid residues of SEQ ID NO: 3 or at least 11 contiguous amino acid residues of SEQ ID NO: 7;
b) a naturally occurring allelic variant of a polypeptide comprising the amino acid sequence of SEQ ID NOs: 3 or 7, wherein the polypeptide is encoded by a nucleic acid molecule which hybridizes to a nucleic acid molecule consisting of the nucleotide sequence of SEQ ID NOs: 1, 2, 4, 5, and 6, or a complement thereof under stringent conditions; and
c) a polypeptide which is encoded by a nucleic acid molecule comprising a nucleotide sequence which is at least 40% identical to a nucleic acid consisting of the nucleotide sequence of SEQ ID NOs: 1, 2, 4, 5, and 6, or a complement thereof.

9. The isolated polypeptide of claim 8 having the amino acid sequence of SEQ ID NOs: 3 or 7.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of SEQ ID NOs: 3 or 7;
b) a polypeptide comprising a fragment of the amino acid sequence of SEQ ID NOs: 3 or 7, wherein the fragment comprises at least 10 contiguous amino acid residues of SEQ ID NO: 3 or at least 11 contiguous amino acid residues of SEQ ID NO: 7; and
c) a naturally occurring allelic variant of a polypeptide comprising the amino acid sequence of SEQ ID NOs: 3 or 7, wherein the polypeptide is encoded by a nucleic acid molecule which hybridizes to a nucleic acid molecule consisting of the nucleotide sequence of SEQ ID NOs: 1, 2, 4, 5, and 6, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for an activity characteristic of the polypeptide.

21. A method for modulating the activity of a polypeptide selected from the group consisting of a polypeptide of claim 8 and a KIAA0880-related polypeptide, the method comprising contacting the polypeptide or a cell expressing the polypeptide with a compound which binds with the polypeptide in a sufficient concentration to modulate the activity of the polypeptide.

22. A method for identifying a compound which modulates the activity of a polypeptide selected from the group consisting of a polypeptide of claim 8 and a KIAA0880-related polypeptide, the method comprising:
a) contacting the polypeptide with a test compound; and
b) determining the effect of the test compound on the activity of the polypeptide to thereby identify a compound which modulates the activity of the polypeptide.

23. An antibody substance which selectively binds with the polypeptide of claim 8, wherein the antibody substance is made by providing the polypeptide to an immunocompetent vertebrate and thereafter harvesting blood or serum from the vertebrate.

24. A method of treating a patient afflicted with a disorder associated with aberrant activity or expression of a protein selected from the group consisting of 65h2, 593, and KIAA0880, the method comprising administering to the patient a compound which modulates the activity of the protein in an amount effective to modulate the activity of the protein in the patient, whereby at least one symptom of the disorder is alleviated.

25. A method of treating a patient afflicted with a disorder associated with aberrant activity or expression of a protein selected from the group consisting of 65h2, 593, and KIAA0880, the method comprising administering to the patient, in an amount effective to modulate the activity of the protein in the patient, a compound selected from the group consisting of
i) the protein;
ii) a variant of the protein;
iii) a nucleic acid encoding the protein; and
iv) an antisense nucleic acid which is capable of annealing with either of an mRNA encoding the protein and a portion of a genomic DNA encoding the protein,
whereby at least one symptom of the disorder is alleviated.

26. A method of diagnosing a disorder associated with aberrant expression of a protein selected from the group consisting of 65h2, 593, and KIAA0880 in a patient, the method comprising assessing the level of expression of the gene encoding the protein in the patient and comparing the level of expression of the gene with the normal level of expression of the gene in a human not afflicted with the disorder, whereby a difference between the level of expression of the gene in the patient and the normal level is an indication that the patient is afflicted with the disorder.

27. A method of treating a patient afflicted with a disorder related to a protein selected from the group consisting of 65h2, 593, and KIAA0880, the method comprising administering to the patient a compound which modulates the activity of the protein in an amount effective to modulate the activity of the protein in the patient, whereby at least one symptom of the disorder is alleviated.

28. A method of treating a patient afflicted with a disorder related to a protein selected from the group consisting of 65h2, 593, and KIAA0880, the method comprising administering to the patient, in an amount effective to modulate the activity of the protein in the patient, a compound selected from the group consisting of
i) the protein,
ii) a variant of the protein;
iii) a nucleic acid encoding the protein; and
iv) an antisense nucleic acid which is capable of annealing with either of an mRNA encoding the protein and a portion of a genomic DNA encoding the protein,
whereby at least one symptom of the disorder is alleviated.

29. A method of diagnosing a disorder related to a protein selected from the group consisting of 65h2, 593, and KIAA0880 in a patient, the method comprising assessing the level of expression of the gene encoding the protein in the patient and comparing the level of expression of the gene with the normal level of expression of the gene in a human not afflicted with the disorder, whereby a difference between the level of expression of the gene in the patient and the normal level is an indication that the patient is afflicted with the disorder.
